(19) United States
(12) Patent Application Publication
(10) Pub. No.: US 2024/0417687 A1
(43) Pub. Date: Dec. 19, 2024

(54) THYMIC CELLS AND METHODS OF MAKING

(71) Applicants: Thymmune Therapeutics, Inc., Cambridge, MA (US); Thymmune Therapeutics, Inc., Cambridge, MA (US)

(72) Inventors: Bing Lim, Cambridge, MA (US); Behzad Gerami-Naini, Cambridge, MA (US); Qiumei Du, Cambridge, MA (US); Stan Wang, Cambridge, MA (US)

(21) Appl. No.: 18/700,649

(22) PCT Filed: Oct. 13, 2022
(86) PCT No.: PCT/US2022/046551

§ 371 Date: Apr. 11, 2024

Related U.S. Application Data
(60) Provisional application No. 63/388,407, filed on Jul. 12, 2022.
(60) Provisional application No. 63/321,136, filed on Mar. 18, 2022.
(60) Provisional application No. 63/296,251, filed on Jan. 4, 2022.
(60) Provisional application No. 63/256,443, filed on Oct. 15, 2021.

(51) Int. Cl. C12N 5/078; A61K 35/26

(52) U.S. Cl. CPC C12N 5/065; A61K 35/26; C12N 2500/38; C12N 2501/119; C12N 2501/15; C12N 2501/155; C12N 2501/16; C12N 2501/2322; C12N 2501/385; C12N 2501/41; C12N 2501/415; C12N 2501/727; C12N 2513/00

(57) ABSTRACT

The present disclosure provides methods for generating thymic cells by the differentiation of pluripotent stem cells. Compositions and systems of cell populations that include thymic cells are also provided herein. Methods of the disclosure also include methods of maintaining thymic cells and methods of treatment using the thymic cells of the disclosure.

### CROSS REFERENCE TO RELATED APPLICATIONS

This application claims benefit of priority under 35 U.S.C. 119(e) to U.S. Provisional Application No. 63/256,443 filed on Oct. 15, 2021; U.S. Provisional Application No. 63/296,251 filed on Jan. 4, 2022; U.S. Provisional Application No. 63/321,136 filed on Mar. 18, 2022; and U.S. Provisional Application No. 63/388,407 filed on Jul. 12, 2022, the contents of which are herein incorporated by reference in their entirety.

### STATEMENT REGARDING GOVERNMENT FUNDING

This invention was made with government support under Grant No. 1R44AI170266-01, awarded by the National Institutes of Health (NIH), National Institute of Allergy and Infectious Diseases (NIAID) Small Business Innovation Research (SBIR). The government has certain rights in the invention.

### BACKGROUND

The thymus is a primary lymphoid organ that plays a central role in the immune system. The microenvironment of the thymus provides a unique training ground for the development of maturation of effector cells such as lymphocytes (e.g., T cells). It is well known in the art that the complex interactions between thymic cells and effector cells can determine the phenotype and functionality of the effector cells. Some thymic cell-effector cell interactions are tuned such that recognition of factors expressed by the thymic cells promotes the survival of the effector cells. In contrast, other thymic cell-effector cell interactions can result in the death of the effector cells. By controlling such interactions, the thymus plays a pivotal role in establishing a repertoire of effector cells that are able to mount an activated immune response to foreign invaders while establishing tolerance to self.

There remains a need for improved methods of generating thymic cells and for cell populations enriched in functional thymic cells that can differentiate into functional thymic epithelial cells.

### SUMMARY

The disclosure provides thymic cells, methods of making thymic cells and/or maintaining thymic cells in culture.

The present disclosure provides methods for inducing differentiation of pluripotent stem cells into thymic cells. Such methods can include the steps of differentiating the pluripotent stem cells into definitive endoderm cells. DE cells can be cultured and differentiated into anterior foregut endoderm (AFE) cells by contacting or incubating the DE cells with BMP inhibitor, a TGFβ inhibitor, an FGF, an ascorbic acid and/or a combination thereof. AFE cells can be cultured and differentiated into ventral pharyngeal endoderm (VPE) cells by culturing in a first VPE medium, and/or a second VPE medium. The first VPE medium can include ascorbic acid, a retinoic acid, an FGF, and/or a TGFβ inhibitor. In some embodiments, first VPE media can further include a WNT inhibitor. The second VPE medium can include Noggin, a WNT activator, an FGF, a retinoic acid, and/or an ascorbic acid. In some embodiments, the second VPE media can further include a BMP inhibitor, an SHH inhibitor, or a combination thereof. The VPE cells can be further differentiated into thymic cells such as thymic epithelial progenitor cells (TEPs) by contacting or incubating the VPE cells with an ascorbic acid, an FGF, a BMP, and/or a WNT activator.

In some embodiments, the thymic cells can be thymic epithelial progenitors (TEPs) and/or thymic epithelial cells (TECs). TEPs can be further differentiated into TECs by culturing the TEPs with an Interleukin, a WNT activator, a RANKL, an FGF, a BMP, and/or an ascorbic acid.

In some embodiments, pluripotent stem cells can be differentiated to DE cells by contacting or culturing the pluripotent stem cells in a first growth medium, containing Activin A, PI-103, and/or CHIR99021. The differentiation to DE cells can further include culturing cells in a second growth medium containing Activin A, a BMP inhibitor, PI-103, and/or CHIR99021.

In some embodiments, BMP inhibitor can be LDN193189. In some embodiments, the TGFβ inhibitor can be SB431542. In some embodiments, FGF can be FGF8b, FGF7, FGF10, FGF1, bFGF or a combination thereof. In some embodiments, the WNT activator can be CHIR99021. In some embodiments, the BMP can be BMP2, BMP4 or a combination thereof. In some embodiments, the interleukin can be IL22. In some embodiments, the WNT inhibitor can be IWR-1. In some embodiments, the BMP inhibitor can be LDN193189. In some embodiments, the SHH inhibitor can SANT-1.

Pluripotent stem cells, the DE cells, the AFE cells, the VPE cells or thymic cells of the disclosure can be cultured in suspension. In some embodiments, the pluripotent stem cells, the DE cells, the AFE cells, the VPE cells or thymic cells can be cultured as aggregates in suspension.

In some embodiments, the pluripotent stem cells, the DE cells, the AFE cells, the VPE cells or thymic cells can be attached to a solid substrate. In some embodiments, the pluripotent stem cells, the DE cells, the AFE cells, the VPE cells or thymic cells can be attached to a solid substrate that includes an extracellular matrix-based medium.

The methods of the disclosure can be performed for about 15 days to 30 days. In some embodiments, the methods can be performed for about 18 days to 25 days. In some embodiments, the pluripotent stem cells can be differentiated into definitive endoderm cells for about 5 days. In some embodiments, the DE cells can be differentiated into AFE cells for from about 2 days to about 3 days. In some embodiments, the AFE cells can be differentiated into VPE cells in the first VPE media for about 2 to 4 days and in the second VPE media for from about 2 days to 3 days. In some embodiments, the VPE cells can be differentiated into thymic cells for about 3 days to 12 days.

The present disclosure also provides thymic cells e.g., TEPs and TECs generated by the methods described herein.

Also provided herein are methods culturing thymic cells in vitro. Such methods can include culturing or incubating the thymic cells in a thymic cell medium that includes FGF10, BMP4, FGF8b, CHIR99021, and/or ascorbic acid. In some embodiments, the thymic cell medium can further include FGF7 and RANKL The methods can also include culturing thymic cells in suspension. As a non-limiting example, the thymic cells can be cultured as aggregates in suspension.

In some embodiments, the present disclosure provides methods of increasing FOXN1 expression in a population of thymic cells. Such methods can include freezing the population of thymic cells, thawing the population of thymic cells, and measuring and comparing the expression of FOXN1 in the population of thymic cells prior to freezing and comparing with FOXN1 expression after thawing the population of thymic cells. In some embodiments, the expression of FOXN1 can be increased by from about 10-fold to 100 fold. The population of thymic cells can be cultured as aggregates in suspension.

Also provided herein are pharmaceutical compositions that include a population of thymic cells prepared by the methods described herein. The present disclosure also provides method of treating or preventing a condition in a subject by administering the pharmaceutical compositions of the disclosure. In some embodiments, the condition is associated with an absence, decline, or aberrant functioning of the thymus in the subject. The condition can be an immunodeficiency, a cancer, an autoimmune disease, an infectious disease or graft versus host disease (GvHD). In some embodiments, the pharmaceutical compositions are administered parenterally. For example, the compositions can be implanted or injected into one or more lymph nodes of the subject.

### BRIEF DESCRIPTION OF THE DRAWINGS

The foregoing and other objects, features and advantages will be apparent from the following description of particular embodiments of the disclosure, as illustrated in the accompanying drawings. The drawings are not necessarily to scale; emphasis instead being placed upon illustrating the principles of various embodiments of the disclosure.

FIG. 1 shows Culture Group 1 to 4 (G1-G4) with increasing frequency of expression level of key biomarkers of definitive endoderm (DE) (SOX17 and FOXA2).

FIG. 2 shows the effect of growth factor BMP4 on expression of developmental genes.

FIG. 3 shows analysis of TEC biomarkers after plating onto Matrigel.

FIG. 4 shows TEC biomarker analysis after freeze-thaw cycle.

FIGS. 5A-5D. FIG. 5A shows HOXA3 expression in different culture conditions. FIG. 5B shows PAX1 expression in different culture conditions. FIG. 5C shows PSMB11 expression in different culture conditions. FIG. 5D shows FOXN1 expression in different culture conditions.

FIGS. 6A-6B. FIG. 6A shows expression of FOXN1 using primers targeting the coding region (left panel), the non-coding region (middle panel) and primers targeting the HA tag of the exogenously expressed FOXN1 (right panel). FIG. 6B shows expression of FOXN1 target genes in mock transfected, or FOXN1 targeted cells one day (D1) or two days (D2) after transfection.

FIG. 7 shows expression of FOXN1 in derivations of iPSC derived thymic cells.

FIG. 8 shows expression of FOXN1 in derivations of Experiment 29 and Experiment 30.

FIG. 9 shows expression of FOXN1 in thymic cells after freezing and thawing the cells.

FIGS. 10A-10E. FIG. 10A and FIG. 10B show the percentage of CD8 positive cells in CD45 positive cells in mice receiving thymic cell transplants. FIG. 10C, FIG. 10D and FIG. 10E, show the percentage of different hematopoietic cells in mice receiving thymic cell transplants at week 3, weeks 11-13 and weeks 14-16 respectively.

FIG. 11 shows sorted fractions of thymic cells that were analyzed by qPCR for various markers relative to GAPDH.

FIGS. 12A-12B. FIG. 12A shows gene expression distributions of iPSC derived thymic cells. FIG. 12B shows quantification of percentage of FOXN1+ cells, KRT8+ cells and EPCAM+ cells.

FIG. 13 is a histogram showing the frequency of CD8⁺ or CD4⁺ cells in peripheral blood.

FIG. 14 shows varying levels of FOXN1 expression in different populations of thymic cells.

FIG. 15 shows HOXA3 and Pax9 expression in cells at the VPE stage.

### DETAILED DESCRIPTION

### I. Introduction

Thymic epithelial cells are important in T cell differentiation. Thymic cells prepared as described herein can permit exploitation of thymus tissue and thymus cell properties, e.g., thymus-related immune functions, for therapeutic applications. For example, it is known that age-related decline in immune function is caused by changes in the composition and functional capabilities of thymic cells. In addition, changes in sex hormones, including androgens and estrogens, cause the thymus itself to atrophy or become senescent. The onset of thymic atrophy can begin as early as the onset of puberty. Thus, regeneration of thymic epithelial cells can provide for compositions and methods that mitigate age-related decline in immune function.

### II. Compositions

### Cells

Cells of the disclosure can include, without limitation, thymic cells, effector cells, pluripotent stem cells, populations thereof and cells derived therefrom.

In some embodiments, cells of the present disclosure can be autologous, allogeneic, syngeneic or xenogeneic in relation to a particular individual or subject. In some embodiments, the thymic cells can be autologous, allogeneic, syngeneic, or xenogeneic in relation to subjects ultimately benefiting from their clinical application. In some embodiments, the cells of the disclosure can be mammalian cells, particularly, human cells. Cells can be primary cells or immortalized cell lines. In some aspects, the cells of the disclosure can be prepared or derived from syngeneic cell sources. Any of the cells described herein can be characterized by markers known in the art for that cell type.

### Thymic Cells

A thymic cell can be a cell with one or more phenotypic or genotypic markers associated with a cell derived from the thymus or a cell destined to become a cell of the thymus. The thymus can be an embryonic, a fetal or an adult thymus.

Thymic cells can be or can be derived from TECs. During embryonic development TECs can be derived from non-hematopoietic cells which are negative for CD45 expression and positive for epithelial marker EpCAM. TECs can be cortical thymic epithelial cells (cTECs) and/or medullary thymic epithelial cells (mTECs). mTECs are characterized by cytokeratin 5 (K5) and cytokeratin 14 (K14) expression but low level of cytokeratin 8 (K8) expression, whereas cTECs express K8 and K18. In some embodiments, thymic cells can be derived from TECs that express both K5 and K8 (K5+K8+). In some aspects, K5+K8+ cells can be progenitors for mTECs and/or cTECs. mTECs can also be positive for the expression of Ulex europaeus agglutinin-1 (UEA-1) on cell surface, but not Ly51 (e.g., UEA-1+Ly51−), while cTECs can be UEA-1-Ly51+. In some embodiments, thymic cells can be or can be derived from mTECs. In some embodiments, mTECs can have high expression of markers such as, but not limited to, cytokeratin 5, cytokeratin 14, UEA-1, CD80, Cathepsin L, and/or Cathepsin S. In some embodiments, thymic cells can be or can be derived from cTECs that have high expression of markers such as, but not limited to, cytokeratin 8, cytokeratin 18, Ly51, CD205, Cathepsin L and/or thymus-specific serine proteases. As a non-limiting example, thymic cells can be or can be derived from cTECs that express markers such as, CCL25, and/or KRT5. mTECs can express markers such as, CCL19, KRT8, and/or AIRE.

In some embodiments, thymic cells can be or can be derived from TECs that express one or more markers such as, FOXN1, PAX9, PAX1, DLIA, ISL1, EYA1, SIX1, IL7, K5, K8 and AIRE.

Thymic cells can be or can be derived from any of the cell types described by Park et al. 2020 Science Vol. 367, Issue 6480 (the contents of which are herein incorporated by reference in its entirety). For example, thymic cells can be derived from myoid cells, e.g., MYOD1 and MYOG expressing myoid cells (herein referred to as TEC(myo)) and/or from NEUROD1, SYP, CHGA-expressing TECS (herein referred to as TEC(neuro)).

Thymic cells can be derived from the cell types described by Bautista et al. 2021 Nat Commun 12, 1096; the contents of which are herein incorporated by reference in its entirety. Thymic cells can be “cTEC^{lo}” cells described by Bautista et al. 2021 and can be characterized by lower levels of functional genes (HLA class II) and containing more KI67⁺-proliferating cells. Thymic cells can be derived from “mTEC^{lo}” cells described by Bautista et al. 2021 and characterized by the expression of CLDN4, lower levels of HLA class II, expression of PSMB11, PRSS16, CCL25, and high levels of chemokine CCL21. Thymic cells can be or can be derived from “mTEC^{hi}” cells described by Bautista et al. 2021 and characterized by SPIB, AIRE, FEZF2, higher levels of expression of HLA class II. Thymic cells can be or can be derived from corneocyte-like mTECs described by Bautista et al. 2021 and characterized by the expression of KRT1, and/or IVL. In some embodiments, Thymic cells can be or can be derived from immature TEC (iTEC) described by Bautista et al. 2021, which express canonical TEC identity genes e.g. FOXN1, PAX9, SIX1.

In some embodiments, thymic cells can be or can be derived from TECs that express one or more markers, such as, but not limited to KRT5, KRT8, AIRE, PSMB11, and/or PRSS16.

In some embodiments, thymic cells can be or can be derived from TECs that express one or more markers, such as, but not limited to AIRE, CK5, CK8, CXCL 12, CCL25, DLL4, and/or HLA-DR.

In some embodiments, thymic cells can be prepared from a cell destined to become a thymic cell. During embryonic development pluripotent stem cells can differentiate to become thymic cells through a step wise differentiation process. In vitro, thymic cells can be prepared from the differentiation of pluripotent stem cells that differentiate into thymic stems via one or more of the following steps: PSCs can differentiate and/or can be induced to differentiate into cells resembling the definitive endoderm (DE). Definitive endoderm cells can differentiate and/or can be induced to differentiate into cells resembling the third pharyngeal pouch endoderm (PPE). Definitive endoderm cells and/or PPE cells can differentiate and/or be induced to differentiate into cells resembling the anterior foregut endoderm (AFE). AFE can differentiate and/or be induced to differentiate into cells resembling the third pharyngeal pouch endoderm (PPE). Thymic epithelial progenitor cells (TEPCs) can be generated from PPE cells. TECs can be derived from TEPCS. Each of the cell types described herein can be characterized by one or more markers. In some embodiments, pluripotent stem cells can be associated with the increased expression of markers such as, but not limited to, OCT 4, SOX 2, and/or Nanog. In some embodiments, definitive endodermal cells can be associated with the increased expression of markers such as, but not limited to, SOX17, FOXA2, CXCR4, and/or CER1. In some embodiments, anterior foregut cells (AFE) can be associated with the increased expression of markers such as, but not limited to, FOXA2, SOX2, and/or PAX9. In some embodiments, the third pharyngeal pouch endodermal cells can be associated with the increased expression of markers such as, but not limited to, HOXA3, TBX1, PAX9, EYA1, SIX1, PBX1, and/or PAX1. In some embodiments, thymic epithelial progenitor cells can be associated with the increased expression of markers such as, but not limited to FOXN1, EPCAM, K5, K8, and/or HOXA3. In some embodiments, the thymic cells can be derived from a DE cell, a third PPE cell, an AFE cell, a TEPC, and/or a TEC cell.

### Pluripotent Stem Cells (PSCs)

In some embodiments, the cells of the present disclosure can be derived from pluripotent stem cells.

Pluripotent stem cells have the capacity to give rise to any of the three germ layers: endoderm, mesoderm, and ectoderm. Pluripotent stem cells can comprise, for example, stem cells, e.g., embryonic stem cells, nuclear transfer derived embryonic stem cells, induced pluripotent stem cells (iPSC), etc. The pluripotent stem cells can have a stem cell phenotype including (i) the ability to self-renew and (ii) pluripotency. Pluripotency-associated genes can include, but are not limited to, Oct-3/4, Sox2, Nanog, GDF3, REXI, FGF4, ESGI, DPPA2, DPPA4, hTERT and SSEAI.

Cells described herein can be derived from embryonic stem cells. ES cells can include a cell that (a) self-renews (b) differentiates to produce all cell types in an organism and/or (c) is derived from a developing organism. ES cells can be derived from the inner cell mass of the blastula of a developing organism. ES cells can also be derived from the blastomere generated by single blastomere biopsy (SBB) involving the removal of a single blastomere from the eight-cell stage of a developing organism. ES cells can be characterized by the expression of markers such as, but not limited to SSEA-3, SSEA-4, TRA-1-60, TRA-1-81, and/or Alkaline phosphatase. Methods of generating and characterizing ES cells are known in the art and can be found in, for example, U.S. Pat. Nos. 7,029,913; 5,843,780; 6,200,806 (the contents of each of which are herein incorporated by reference in their entirety).

Induced pluripotent stem cells (iPSCs) can also be used to generate cells of the present disclosure. iPSCs can include cells with one or more properties such as, but not limited to (a) self-renewal (b) ability to differentiate to produce all types of cells in an organism and/or (c) be derived from a somatic cell. iPSCs can express markers such as, but not limited to SSEA3, SSEA4, SOX2, OCT3/4, Nanog, TRA160, TRA1818, TDGF1, Dnmt3b, FoxD3, GDF3, Cyp26a1, TERT, Zpf42. Methods of generating and characterizing iPSC cells can be found in, for example, US Patent Publication Nos. US20090047263, US20090068742, US2009191159, US20090227032, US20090246875, and US20090304646 (the contents of each of which are herein incorporated by reference in their entirety). In some embodiments, the iPSCs can be derived from a T cell or non-T cell, a B cell, or any other cell from peripheral blood mononuclear cell, a hematopoietic progenitor cell, or any other somatic cell type.

In some embodiments, pluripotent stem cells can be derived from adult stem cells. Adult stem cells can be obtained from the inner ear, bone marrow, mesenchyme, skin, fat, liver, muscle, and/or blood of a subject such as subject. PSCs can also include embryonic stem cells derived from a placenta or umbilical cord; progenitor cells (e.g., progenitor cells derived from the inner ear, bone marrow, mesenchyme, skin, fat, liver, muscle, and/or blood).

### Effector Cells

An “effector cell” refers to any cell or cell type which, when in contact with or in proximity to a thymic cell, acquires the ability to execute, initiate or propagate a signal or a cell death trigger. “Contact or proximity” can refer to spatiotemporal closeness sufficient to enable cell-intrinsic or cell-extrinsic (e.g., cell-to-cell) signaling or other communication or interaction.

Effector cells described herein can be derived from pluripotent stem cells. In some embodiments, effector cells can be derived from embryonic stem cells, hematopoietic stem or progenitor cells, cells isolated from bone marrow, cord blood, peripheral blood, thymus, or the stem or progenitor cells can have been differentiated from embryonic stem cells (ESC) or induced pluripotent stem cells (iPSC) in vitro. Stem or progenitor cells from primary tissue or ESC or iPSC can be from human or non-human animals (e.g., mouse) in origin.

In some embodiments, the effector cell can be a hematopoietic cell. In some embodiments, the effector cell can be a lymphocyte. In some embodiments, the lymphocyte can be a CD45 positive lymphocyte.

Effector cells can be CD4+CD8− T cells, CD4−CD8+ T cells, CD34+CD7+CD1a+ cells, CD3+ TCRab+ cells, CD3+ TCRgd+ cells, CD3+TCRab+CD4+CD8− cells, CD3+TCRab+CD8+CD4− cells, CD3+TCRab+CD4+CD8− CD45RO− CD45RA+ cells, CD3+TCRab+CD8+CD4− CD45RO− CD45RA+ cells, CD3+TCRab+CD4+CD8− CD45RO− 30 CD45RA+ CCR7+ cells, CD3+TCRab+CD8+CD4− CD45RO− CD45RA+ CCR7+ cells, CD3+TCRab+CD4+CD8− CD45RO− CD45RA+CD27+ cells, CD3+TCRab+CD8+CD4− CD45ROCD45RA+ cells, CD27+, CD34+CD7+CD1a+ cells, CD34+CD5+CD7+ cells, CD34+CD5+CD7− cells, natural killer T cells, regulatory T cells, antigen-specific T cells, intraepithelial lymphocyte T cells, or cells that are CD45+, CDI 1b+, CDI 1b−, CD15+, CD15−, CD24+, CD24−, CDI 14+, CD114−, CD182+, CD182−, CD4+, CD4−, CD14+, CD14−, CD11a+, CD11a−, CD91+, CD91−, CD16+, CD16−, CD3+, CD3−, CD25+, CD25−, Foxp3+, Fox3p−, CD8+, CD8−, CD19+, CD19−, CD20+, CD20−, CD24+, CD24, CD38+, CD38−, CD22+, CD22−, CD61+, CD61−, CD16+, CD16−, CD56+, CD56−, CD3 1+, CD3 1−, CD30+, CD30−, CD38+, and/or CD38− and/or cells that are positive for combinations thereof.

In some embodiments, the effector cell can be a T cell. T cells can be cultured T cells, e.g., primary T cells, or T cells from a cultured T cell line, e.g., Jurkat, SupT1, etc., or T cells obtained from a mammal. If obtained from a mammal, the effector cells can be obtained from numerous sources, including but not limited to blood, bone marrow, lymph node, thymus, spleen, or other tissues or fluids. Effector cells can also be enriched for or purified. The T cells can be any type of T cells and can be of any developmental stage, including but not limited to, CD4+/CD8+ double positive T cells, CD4+ helper T cells, e.g., Th1 and Th2 cells, CD4+ T cells, CD8+ T cells (e.g., cytotoxic T cells), peripheral blood mononuclear cells (PBMCs), peripheral blood leukocytes (PBLs), tumor infiltrating cells (TILs), memory T cells, naïve T cells.

In some embodiments, effector cells can be CCRXA−, CD3+, CD69−, MHC-1+, CD62L+, and/or CCR7+.

Effector cells can have a naïve T cell (TN) phenotype, central memory T cell (TcM) phenotype, or effector memory T cell (TEM) phenotype. The phenotypes of TN, TcM, and TEM cells are known in the art. For example, CCR7 and CD62L are expressed by TN and TcM cells but are not expressed by TEM cells. The transcription factors LEF1, FOXP1, and KLF7 are expressed by TN and TcM cells, but are not expressed by TEM cells. CD45RO and KLRG1 are not expressed by TN cells, but are expressed by TEM cells (Gattinoni et al., Nat. Rev. Cancer, 12: 671-84 (2012)). Alternatively, or additionally, TN and TcM cells can be characterized by longer telomeres as compared to those of TEM cells.

In some embodiments, effector cells can be TCRα+TCRβ+ cells. TCRα+TCRβ+ effector cells can be T cells expressing receptor expressing an alpha (α) chain and/or a beta (β) chain. TCR alpha and beta chains are known in the art.

Effector cell can be further modified. In further embodiments, the stem or progenitor cells can be genetically modified. For example, the stem or progenitor cells can express an exogenous T cell receptor (TCR) or a chimeric antigen receptor (CAR), or both. In further embodiments, the stem or progenitor cells can express an exogenous invariant natural killer T cell (iNKT) associated TCR. In still further embodiment, the stem or progenitor cells express an exogenous antigen-specific TCR or have an exogenous genetic modification of genes that modulate T cell differentiation, expansion or function.

In some embodiments, effector cells can be FOXP3+ Tregs. Tregs can be generated via clonal diversion of mTECs, whereby expression of Aire in mTECs leads to expression of tissue-specific antigens which become surface displayed (i.e., on antigen presenting cells (APCs)). Autoreactive T cells that recognize the tissue-specific antigens give rise to FOXP3+ Tregs that can mediate peripheral tolerance (see Husebye, Eystein S., Mark S. Anderson, and Olle Kampe. “Autoimmune polyendocrine syndromes.” New England Journal of Medicine 378.12 (2018): 1132-1141, incorporated herein by reference in its entirety).

### Supporting Cells

In some embodiments, cells of the present disclosure can include or can be cultured with supporting cells that aid in the generation of thymic cells and/or maintenance of thymic cells in culture. Non-limiting examples of supporting cells include hematopoietic non-T-cell progenitors, such as macrophages and dendritic cells (DCs); non-hematopoietic cells, such as epithelial cells and fibroblasts; stromal cells such as the progenitors of skeletal tissue, components such as bone, cartilage, the hematopoiesis-supporting stroma, and adipocytes. Supporting cells, in some embodiments, encourage the proliferation, survival, maturation, or function of thymic cells. In some embodiments, the supporting cells can be mesenchymal in origin.

Supporting cells can be non-immune cells, that can be present in the thymic microenvironment. For example, the support cells can be fibroblasts, vascular smooth muscle cells (VSMCs), endothelial cells, and/or lymphatic endothelial cells.

In some embodiments, the supporting cells can be neuroendocrine cells (expressing BEX1, NEUROD1), muscle-like myoid (expressing MYOD1, DES), and myelin positive epithelial cells (expressing SOX10, MPZ) described by Bautista et al. 2021 Nat Commun 12, 1096 (2021); the contents of which are herein incorporated by reference in its entirety. In some embodiments, mesenchymal cells can be associated with markers such as, but not limited to LAMA2, LAMA4, PDGFRA, PDGFRB, LUM, CSPG4, COL1A2, COL3A1, IGF1, FGF7, FGF10, FST, BMP4, SFRP2, WNT5A. The mesenchymal cells can be positive or negative for one or more of these markers. In some embodiments, the mesenchymal cells can be positive for some marks described herein but can be negative for other markers.

In some embodiments, endothelial cells can be associated with one or markers such as, but not limited to, VEGFC, PECAMI, APLNR, PROX1, LYVE1, ACKR1, SELE, SELP, FN1, and/or TGFB1. The endothelial cells can be positive or negative for one or more of these markers. In some embodiments, the endothelial cells can be positive for some marks described herein but can be negative for other markers.

### Three-Dimensional Culture

In some embodiments, the cells of the disclosure can be cultured in a three-dimensional culture (3D) system. In 3D culture, cells are cultured with surrounding extracellular framework in three dimensions. Pluripotent stem cells, DE cells, AFE cells, VPE cells, TEPs and/or TECs can be cultured in 3D. Cells of the disclosure can be cultured with or without a supporting scaffold. In some embodiments, cells of the disclosure can be cultured in a scaffold-free 3D cell culture. Cells can be cultured as organoids or spheroids. Cells can be cultured as spheroids. In spheroid culture, cells can be grown as an aggregation of cells into a round cell cluster that is a three-dimensional structure. Cells can be round and uniform in shape. In some embodiments, cells of the disclosure can be cultured as organoids. As used herein, the term organoid refers to an artificial model of live cells in a three-dimensional or multi-layered configuration and can include cells other than thymic cells, e.g., effector cells and supporting cells. In some embodiments, the organoids can form an ordered structure.

In some embodiments, the compositions of the disclosure can include a thymic organoid. An organoid is an in vitro, three-dimensional, miniature recapitulation of an organ. A thymic organoid can be an in vitro three-dimensional, miniature version of the thymic organ that can mimic the physiology and function of a human thymus. Methods of preparing thymic organoids are described in the International Patent Publication WO2019060336, the contents of which are herein incorporated by reference in its entirety.

In some embodiments, effector cells can be prepared by differentiating the pluripotent stem cells or progenitor cells into lymphocytes by culturing PSC or progenitor cells with thymic cells. In some embodiments, the thymic cells can express a Notch ligand. In some embodiments, the Notch ligand can be Delta-like 1 (DLL1). In some embodiments, the Notch ligand is Delta-like 4 (DLL4). In some embodiments, the Notch ligand is one described herein or in the art, such as in U.S. Pat. No. 7,795,404, which is herein incorporated by reference in its entirety. Effector cells of the present disclosure can be prepared using the thymic organoid cell culture systems. In some embodiments, the method further comprises contacting the co-cultured stem or progenitor cells and stromal cells with Flt-3 ligand and/or IL-7 and/or Stem Cell Factor/Kit ligand and/or thrombopoietin. In some embodiments, differentiating the stem or progenitor cell into a T cell comprises: culturing a three dimensional (3D) cell aggregate, comprising: a) a selected population of supporting cells that endogenously or exogenously express a Notch ligand; b) a selected population of stem or progenitor cells; with a serum-free medium comprising B-27® supplement, xeno-free B-27® supplement, GS21™ supplement, ascorbic acid, Flt-3 ligand, IL-7, or a combination thereof. Any of the methods for generating lymphocytes from stem cells or progenitor cells described in International Patent Publication WO2017075389 can be useful in the present disclosure (the contents of which are herein incorporated by reference in their entirety).

In some embodiments, the thymic organoids can be based on the artificial thymic organoids described by Seet C S, et al. Nat Methods. 2017; 14(5):521-530 (the contents of which are herein incorporated by reference in its entirety). To prepare thymic organoids, thymic cells can be harvested by trypsinization and resuspended in serum free culture medium (“RB27”) which can include of RPMI 1640 (Corning, Manassas, VA), 4% B27 supplement (ThermoFisher Scientific, Grand Island, NY), 30 μM L-ascorbic acid 2-phosphate sesquimagnesium salt hydrate (Sigma-Aldrich, St. Louis, MO) reconstituted in PBS, 1% penicillin/streptomycin (Gemini Bio-Products, West Sacramento, CA), 1% Glutamax (ThermoFisher Scientific, Grand Island, NY), 5 ng/ml rhFLT3L and 5 ng/ml rhIL-7 (Peprotech, Rocky Hill, NJ). Different ratios of thymic cells and effector cells can be prepared in 1.5 ml Eppendorf tubes and centrifuged at 300 g for 5 min. at 4° C. in a swinging bucket centrifuge. Supernatants were carefully removed, and the cell pellet was resuspended by brief vortexing. For each organoid, a 0.4 μm Millicell trans well insert (EMD Millipore, Billerica, MA; Cat. PICM0RG50) can be placed in a 6-well plate containing 1 ml RB27 per well. To plate the organoids, inserts were taken out and rested on the edge of plate to drain excess medium. The cell slurry can be adjusted to 5 l per organoid, drawn up in with a 20 μl pipet tip and plated by forming a drop at the end of the pipet tip which was gently deposited onto the cell insert. The cell insert can be placed back in the well containing 1 mL RB27. Medium can be changed completely every 3-4 days by aspiration from around the cell insert followed by replacement with 1 ml with fresh RB27/cytokines. In some embodiments, organoids can be cultured in this manner for up to 10 weeks, 15 weeks, 20 weeks, 25 weeks, or 30 weeks.

At the indicated times, organoids cells were harvested by adding FACS buffer (PBS/0.5% bovine serum album/2 mM EDTA) to each well and briefly disaggregating the organoids by pipetting with a 1 ml “P1000” pipet, followed by passage through a 50 μm nylon strainer. In some experiments, single cell suspensions of MS5-hDLL1 cells were 7-irradiated at the indicated doses prior to use in organoids.

Thymic organoid effector cell co-cultures can be prepared as described in Seet C S, et al. Nat Methods. 2017; 14(5):521-530 (the contents of which are herein incorporated by reference in its entirety). Thymic cells can be seeded into 0.1% gelatin-coated 12 well plates 1-2 days prior to use to achieve 70-80% confluence. Medium can be aspirated from monolayers and 1.5×10⁴ FACS purified effector cells (CD34+CD3− hematopoietic cells) can be plated with thymic organoids in 2 ml of medium composed of MEMα, 20% FBS, 30 μM L-Ascorbic acid, 5 ng/ml rhFLT3L, and 5 ng/ml rhIL-7. In some embodiments, effector cells can be transferred to thymic organoids every 4-5 days by harvesting cells, filtering through a 50 μm nylon strainer, and replating in fresh medium. When confluent, cells were split into multiple wells containing fresh stromal layers.

In some embodiments, thymic cells of the disclosure can be combined with double negative day 14 T cells to form a cluster of cells which can then be deposited onto the trans well as an “organoid” and maintained in an air-liquid interface culture condition. In some embodiments, cells can be harvested from the medium every few days to assess T cell maturation.

### III. Methods

The present disclosure provides methods of differentiating pluripotent stem cells into thymic cells. In some embodiments, the present disclosure provides method of differentiating induced pluripotent stem cells into thymic cells.

In some embodiments, one or more of the steps involved in the differentiation of iPSCs to thymic cells can include the activation of WNT signaling. As a non-limiting example, the activator of WNT signaling can be CHIR99021.

In some embodiments, one or more of the steps involved in the differentiation of iPSCs to thymic cells can include the inhibition of WNT signaling. As a non-limiting example, the inhibitor of WNT signaling can be IWR1 (or IWR-1).

In some embodiments, one or more of the steps involved in the differentiation of iPSCs to thymic cells can include the inhibition of BMP signaling. In some embodiments, the inhibition of BMP signaling can be achieved using BMP pathway inhibitor, LDN193189.

In some embodiments, one or more of the steps involved in the differentiation of iPSCs to thymic cells can include the inhibition of SHH signaling. In some embodiments, the inhibition of SHH is achieved by using an SHH antagonist, SANT-1.

In some embodiments, one or more of the steps involved in the differentiation of iPSCs to thymic cells can include the inhibition TGFβ signaling. In some embodiments, the inhibition of TGFβ signaling is achieved by using TGFβ inhibitor, SB431542.

In some embodiments, one or more of the steps involved in the differentiation of iPSCs to thymic cells can a cell culture medium containing Insulin Transferrin Selenium (ITS), knockout replacement serum (KSR), Penicillin Streptomycin (also referred to herein as “Pen Strep”) and non-essential amino acids (NEAA).

### Preparation and Maintenance of Thymic Cells

Provided herein are methods of differentiating pluripotent stem cells into thymic cells. Such methods can include culturing pluripotent stem cells in a first growth medium, second growth medium or a combination thereof. In some embodiments, the first or second growth medium can include PI-103 (a multitargeted P13K inhibitor). In some embodiments, the first growth medium includes DMEM-F12, Activin A, CHIR99021, insulin transferrin selenium (ITS), and knockout serum replacement (KSR). In some aspects, the second growth medium includes DMEM-F12, bFGF, Activin A, LDN193189, ITS and KSR. In some embodiments, the cells are cultured in the presence of PI-103. The concentration of PI-103 can be from about 1 nM to 1000 nM. In one embodiment, the concentration of PI-103 can be 50 nM.

The definitive endoderm cells can be further cultured and differentiated into anterior foregut cells by contacting or incubating the definitive endoderm cells with at least one of SB431542, LDN-193189 and KSR. In some embodiments, the anterior foregut cells can be cultured and differentiated into pharyngeal endoderm cells by contacting or incubating the anterior foregut cells with at least one of EGF, retinoic acid, FGF8B, and SHH. In some embodiments, the pharyngeal endoderm cells can be cultured and differentiated into, thymic epithelial cells by contacting or incubating the pharyngeal endoderm cells with at least one of BMP4, FGF8b, EGF, SANT, CHIR99021, Ascorbic Acid, or a combination thereof. In some embodiments, the differentiation is performed from about 14 days to 25 days. For example, the differentiation is performed for about 18 days, 19 days, 20 days, 21 days, 22 days, 23 days, 24 days or 25 days.

In some embodiments, the present disclosure provides methods for the preparation of one or more cells or cell types described herein. In some embodiments, the cells can be thymic cells.

An accumulating body of data in public databases provide single cell transcriptomes of primary human and murine thymuses (See Bautista et al. 2021 Nat Commun 12, 1096; Kernfeld, et al. Immunity. 2018 Jun. 19; 48(6):1258-1270.e6; Zeng et al. Immunity. 2019 Nov. 19; 51(5):930-948.e6; the contents of each of which are herein incorporated by reference in its entirety). This provides a rich source of material for identification of factors that promote the differentiation and/or maturation of cells of the disclosure to thymic cells. By analysis of scRNA sequencing data, the present disclosure identifies potential factors and/or supporting cells that can promote and/or maintain thymic cell phenotype.

In some embodiments, cells of the present disclosure can be isolated from an organism. In some embodiments, the organism can be a mammal. Mammalian cells can be isolated from human, rodent, porcine and/or bovine sources. Human sources of cells of the disclosure can be autologous or allogeneic. In some embodiments, the tissues that contain the cells of the disclosure can be harvested and used as such for applications described herein. Cells of the disclosure can be obtained from embryonic, fetal, adult organism. In some aspects, the organism can be alive or can be a cadaver organism.

Cells described herein can be derived from other cell types. As a non-limiting example, the cells of the disclosure can be derived from pluripotent stem cells (PSCs). In some embodiments, the cells of the disclosure can be derived from progenitor cells. In some embodiments, cells of the disclosure can be derived by the differentiation of PSCs and/or progenitor cells.

In some embodiments, thymic cells can be prepared from PSCs. In this regard, the methods can comprise culturing the pluripotent stem cells for a time and under conditions sufficient to differentiate the pluripotent stem cells into thymic cells. For example, the method can comprise culturing the pluripotent stem cells in the presence of the factors and/or inhibitors that drive the differentiation of PSCs to thymic cells. Methods for differentiating PSCs into thymic cells are known in the art. The methods for differentiating PSCs into thymic cells can include the use of one or more parameters know in the art for differentiation or combinations thereof. The parameters include, but are not limited to, (i) factors promoting differentiation (ii) inhibitors promoting differentiation (iii) duration of time for promoting differentiation (iv) temperature (v) substrate and/or (vi) supporting cells that promote differentiation. Any of the methods or parameters for differentiating PSCs into thymic cells described in following references can be used herein and include Parent et al. Cell Stem Cell. 2013 Aug. 1; 13(2):219-29; Soh et al. Stem Cell Rep. 2014 Vol. 2 j 925-937; Sun et al. Cell Stem Cell. 2013 Aug. 1; 13(2):230-6; Okabe et al. Cell. Reprog. 2015 Vol 17, No. 5; Su et al. Sci. Rep. 2015 5, 9882; Otsuka et al. Sci Rep 2020 10:224; International Patent Publications, WO2019060336, WO2020205859, WO2020220040, WO2014134213, WO2010143529, WO2011139628; and Chinese Patent Publication CN201110121243; the contents of each of which are herein incorporated by reference in their entirety.

### Preparation and Maintenance of Definitive Endoderm Cells

The present disclosure provides method for preparing definitive endoderm cells which can subsequently be differentiated into thymic cells. In some embodiments, the definitive cells can be prepared by culturing cells in two-dimensional culture or three-dimensional culture. Such methods can include culturing pluripotent stem cells in a first growth medium, second growth medium or a combination thereof. In some embodiments, the first or second growth medium can include PI-103 (a multitargeted P13K inhibitor). In some embodiments, the first growth medium includes Activin A, CHIR99021, insulin transferrin selenium (ITS), and/or knockout serum replacement (KSR).

In some aspects, the second growth medium includes basic fibroblast growth factor (bFGF), Activin A, LDN193189, ITS and KSR. In some embodiments, the second growth medium includes CHIR99021.

In some embodiments, the concentration of CHIR99021 is from about 0.1 μM to 100 μM. In some embodiments, the concentration of CHIR99021 is about 2 μM-3 μM.

In some embodiments, the cells are cultured in the presence of PI-103. The concentration of PI-103 can be from about 1 nM to 1000 nM. In one embodiment, the concentration of PI-103 can be 50 nM. In some aspects, the concentration of PI-103 can be 25 nM. In some embodiments, the pluripotent stem cells can be cultured for about three to five days. The PI-103 can be added for 1-2 days. The pluripotent stem cells can be embryonic stem cells or induced pluripotent stem cells.

In some embodiments, the pluripotent stem cells can be cultured for about three to five days. Stem cells can be cultured in the first growth medium for about one to two days and in the second growth medium for about two to three days. The pluripotent stem cells can be cultured in the first growth medium for two days and in the second growth medium for three days. In some embodiments, the concentration of Activin A can be about 100 ng/ml. In some embodiments, the concentration of CHIR99021 can be 2 μM. In some embodiments, the concentration of bFGF can be 10 ng/ml. In some embodiments, the concentration of LDN193189 can be 200 nM. In some embodiments, CHIR99021 can be added to the second growth medium for about one day.

Provided herein are methods of differentiating pluripotent stem cells into thymic cells. Such methods can include culturing pluripotent stem cells in a first growth medium, second growth medium or a combination thereof. In some embodiments, the first or second growth medium can include PI-103 (a multitargeted P13K inhibitor). In some embodiments, the first growth medium includes DMEM-F12, Activin A, CHIR99021, insulin transferrin selenium (ITS), and knockout serum replacement (KSR). In some aspects, the second growth medium includes DMEM-F12, bFGF, Activin A, LDN193189, ITS and KSR. In some embodiments, the second growth medium includes CHIR99021. In some embodiments, the concentration of CHIR99021 is from about 0.1 μM to 100 μM. In some embodiments, the concentration of CHIR99021 is 2 μM. In some embodiments, the cells are cultured in the presence of PI-103. The concentration of PI-103 can be from about 1 nM to 1000 nM. In one embodiment, the concentration of PI-103 can be 50 nM.

### Preparation of Anterior Foregut Endodermal (AFE) Cells

The definitive endoderm cells can be further cultured and differentiated into anterior foregut cells. In some embodiments, the AFE cells can be prepared by culturing cells in two-dimensional culture or three-dimensional culture. Definitive endoderm cells can be differentiated into AFE cells by contacting DE cells with a BMP inhibitor, a TGFβ inhibitor, at least one FGF, and/or Ascorbic acid. In some embodiments the cell culture medium utilized for the differentiation of DE cells to AFE cells can include N2-supplement (GIBCO, Waltham, Massachusetts), Basal Medium Eagle (BME), GLUTAMAX (GIBCO, Waltham, Massachusetts), B27™ serum-free supplement, non-essential amino acids, KSR and/or ITS.

In some embodiments the BMP inhibitor can be LDN193189. In some embodiments, the concentration of LDN193189 is from about 0.1 nM to about 1000 nM. In some aspects, the concentration of LDN193189 is from about 100 to 200 nM.

In some embodiments, the TGFβ inhibitor can be SB431542. In some embodiments, the concentration of SB431542 is from about 1 μM to about 100 μM. As a non-limiting example, the concentration of SB431542 is 10 μM.

In some embodiments, the FGF can be FGF8. In some embodiments, the concentration of FGF8 is from about 1 ng/ml to about 100 ng/ml. As a non-limiting example, the concentration of FGF8b is about 25-50 ng/ml.

DE cells can be differentiated to AFE cells for about 1 day, 2 days, 3 days, 4 days or 5 days.

### Preparation of Ventral Pharyngeal Endoderm (VPE) Cells

Differentiation of AFE to VPE cells is performed as a single step process or as a multistep process. The multi-step process can be a two-step process. In a first step, the AFEs are cultured in a VPE1 media and in the second step, the cells are cultured in a VPE2 media. In some embodiments, the VPE cells can be prepared by culturing cells in two-dimensional culture or three-dimensional culture. The VPE1 step can include culturing cells for about 1 day, 2 days, 3 days, 4 days or 5 days. The VPE2 step can include culturing cells for about 2 days, 3 days, 4 days, 5 days, or 6 days.

In some embodiments, the VPE1 media can include Retinoic Acid, at least one FGF, a WNT inhibitor, TGFβ inhibitor, and/or Ascorbic acid.

In some embodiments, the VPE2 media can include Noggin, BMP inhibitor, WNT activator (e.g., CHIR99021), at least one FGF, Retinoic Acid, an SHH antagonist, and/or Ascorbic acid.

In some embodiments, the FGF can be FGF8, FGF7, and/or FGF10. In some embodiments, the concentration of FGF8 is from about 1 ng/ml to about 100 ng/ml. As a non-limiting example, the concentration of FGF8b is about 25-50 ng/ml.

In some embodiments, the WNT inhibitor is IWR1. The concentration of IWR1 can be from about 0.01 to 10 μM. As a non-limiting example, the concentration of IWR1 is 2.5 μM.

In some embodiments, the TGFβ inhibitor can be SB431542. In some embodiments, the concentration of SB431542 is from about 1 μM to about 100 μM. As a non-limiting example, the concentration of SB431542 is 10 μM.

In some embodiments, the concentration of Ascorbic Acid is from about 0.1 to 30 μM. As a non-limiting example, the concentration of Ascorbic Acid can be 10 μM.

In some embodiments the BMP inhibitor can be LDN193189. In some embodiments, the concentration of LDN193189 is from about 0.1 nM to about 1000 nM. In some aspects, the concentration of LDN193189 is from about 100 to 200 nM.

In some embodiments, the SHH inhibitor can be SANT-1. In some embodiments, the concentration of SANT-1 is from about 0.01 μM to about 10 μM. As a non-limiting example, the concentration of SANT-1 is 0.25 μM.

In some embodiments, the anterior foregut cells can be cultured and differentiated into pharyngeal endoderm cells by contacting or incubating the anterior foregut cells with at least one of EGF, retinoic acid, FGF8B, and/or SHH.

In some embodiments the VPE1 and/or VPE2 media can include N2-supplement (GIBCO, Waltham, Massachusetts), Basal Medium Eagle (BME), GLUTAMAX (GIBCO, Waltham, Massachusetts), B27™ serum-free supplement (with or without Vitamin A), non-essential amino acids, KSR and/or ITS.

### Preparation of Thymic Epithelial Progenitor Cells (TEP) Cells

Differentiation of VPE cells to TEP cells can be performed by culturing the cells in a TEP media. In some embodiments, the TEP cells can be prepared by culturing cells in two-dimensional culture or three-dimensional culture. The TEP step can include culturing cells for about 1 day, 2 days, 3 days, 4 days, 5 days, or 6 days.

In some embodiments, the VPE cells can be differentiated into TEP cells using BMP (e.g., BMP4, BMP2), a WNT activator e.g., CHIR99021, at least one FGF, and/or Ascorbic acid.

In some embodiments the TEP media can include N2-supplement (GIBCO, Waltham, Massachusetts), Basal Medium Eagle (BME), GLUTAMAX (GIBCO, Waltham, Massachusetts), B27™ serum-free supplement (with or without Vitamin A), non-essential amino acids, KSR and/or ITS.

In some embodiments, the BMP can be BMP2 or BMP4. The concentration of BMP can be 1 ng/ml to about 100 ng/ml. In some aspects, the concentration of BMP can be 50 ng/ml.

In some embodiments, the FGF can be FGF8, FGF7, FGF1, and/or FGF10. In some embodiments, the concentration of FGF is from about 1 ng/ml to about 100 ng/ml. As a non-limiting example, the concentration of FGF is about 25-50 ng/ml.

In some embodiments, the pharyngeal endoderm cells can be cultured and differentiated into, thymic epithelial cells by contacting or incubating the pharyngeal endoderm cells with at least one of BMP4, FGF8b, EGF, SANT-1 (SHH antagonist), CHIR99021, Ascorbic Acid, or a combination thereof.

### Preparation of Thymic Epithelial Cells (TEC)

TEP cells can be further differentiated in vitro into TECs. Differentiation to TECs can be performed in 2D or 3D culture. In some embodiments, the differentiation of TEPs can be performed for about 2 days, 3 days, 4 days, 5 days or 6 days.

In some embodiments, the differentiation of TEPs to TECs is carried out in a TEC medium.

TEC medium can include RANKL, Interleukin e.g. (IL22), at least one FGF, at least one BMP (e.g., BMP4), a WNT activator, and/or ascorbic acid.

In some embodiments, the concentration of RANKL can be from about 1 ng/ml to about 100 ng/ml. In some aspects, the concentration of RANKL can be from about 20 ng/ml to about 50 ng/ml.

In some embodiments, the FGF can be FGF8, FGF7, FGF1, and/or FGF10. In some embodiments, the concentration of FGF is from about 1 ng/ml to about 100 ng/ml. As a non-limiting example, the concentration of FGF is about 25-50 ng/ml.

In some embodiments, the concentration of interleukins is from about 1 ng/ml to about 100 ng/ml. As a non-limiting example, the concentration of IL22 is about 20 ng/ml.

The TEC medium can include N2-supplement (GIBCO, Waltham, Massachusetts), Basal Medium Eagle (BME), GLUTAMAX (GIBCO, Waltham, Massachusetts), B27™ serum-free supplement (with or without Vitamin A), non-essential amino acids, KSR and/or ITS.

In some embodiments, the differentiation is performed from about 14 days to seventeen days. In some embodiments, the cells of the disclosure can be cultured as aggregates. In some aspects, the cells disclosure can be cultured in an extracellular matrix-based medium e.g., Geltrex.

### Preparation of Effector Cells

Also provided herein are methods of preparing effector cells. In some embodiments, the effector cells can be lymphocytes. Effector cells can be obtained from primary cells from a mammal or from an established cell line. If obtained from a mammal, the effector cells can be obtained from numerous sources, including but not limited to blood, bone marrow, lymph node, thymus, spleen, or other tissues or fluids. Effector cells can also be enriched for or purified. In some embodiments, the effector cells can be T cells. The T cells can be any type of T cells and can be of any developmental stage, including but not limited to, CD4+/CD8+ double positive T cells, CD4+ helper T cells, e.g., Th1 and Th2 cells, CD4+ T cells, CD8+ T cells (e.g., cytotoxic T cells), peripheral blood mononuclear cells (PBMCs), peripheral blood leukocytes (PBLs), tumor infiltrating cells (TILs), memory T cells, naïve T cells. Methods of isolating and/or enriching lymphocytes are known in the art. Methods of enriching a population of lymphocytes obtained from a mammal or a donor can be accomplished by any suitable separation method including, but not limited to, the use of a separation medium (e.g., FICOLL-PAQUE™, ROSETTESEP™ HLA Total Lymphocyte enrichment cocktail, Lymphocyte Separation Medium (LSA) (MP Biomedical Cat. No. 0850494×), or the like), cell size, shape or density separation by filtration or elutriation, immunomagnetic separation (e.g., magnetic-activated cell sorting system, MACS), fluorescent separation (e.g., fluorescence activated cell sorting system, FACS), and/or bead-based column separation.

In some embodiments, effector cells described herein can be derived from pluripotent stem cells. In some embodiments, effector cells can be derived from embryonic stem cells, hematopoietic stem or progenitor cells, cells isolated from bone marrow, cord blood, peripheral blood, thymus, or the stem or progenitor cells can have been differentiated from embryonic stem cells (ESC) or induced pluripotent stem cells (iPSC) in vitro. Stem or progenitor cells from primary tissue or ESC or iPSC can be from human or non-human animals (e.g., mouse) in origin.

In some embodiments, effector cells can be prepared differentiating the pluripotent stem cells or progenitor cells into lymphocytes by culturing PSC or progenitor cells with supporting cells that ectopically express a Notch ligand. In some embodiments, the supporting cells can be OP9 cells. In some embodiments, the Notch ligand is Delta-like 1 (DLL1). In some embodiments, the Notch ligand is Delta-like 4 (DLL4). In some embodiments, the Notch ligand is one described herein or in the art, such as in U.S. Pat. No. 7,795,404, which is herein incorporated by reference in its entirety. Effector cells of the present disclosure can be prepared using the Artificial Thymic Organoid (ATO) cell culture system which utilizes supporting cells that ectopically express OP9-DLL1. In some embodiments, the method further comprises contacting the co-cultured stem or progenitor cells and stromal cells with Flt-3 ligand and/or IL-7 and/or Stem Cell Factor/Kit ligand and/or thrombopoietin. In some embodiments, differentiating the stem or progenitor cell into a T cell comprises: culturing a three dimensional (3D) cell aggregate, comprising: a) a selected population of supporting cells that express an exogenous Notch ligand; b) a selected population of stem or progenitor cells; with a serum-free medium comprising B-27® supplement, xeno-free B-27® supplement, GS2 1™ supplement, ascorbic acid, Flt-3 ligand, IL-7, or a combination thereof. Any of the methods for generating lymphocytes from stem cells or progenitor cells described in International Patent Publication WO2017075389 can be useful in the present disclosure (the contents of which are herein incorporated by reference in their entirety).

In some embodiments, the effector cell can be or can be derived from a hematopoietic cell. Methods of preparing hematopoietic cells from pluripotent stem cell are known in the art, for example as described in U.S. Pat. No. 9,834,754 and can include one or more of the following steps such as (i) inducing hematopoietic differentiation in a population of human pluripotent stem cells, wherein activin/nodal signaling is inhibited between day 1 and day 4 of differentiation; (ii) sorting the induced population based on expression of CD34 and CD43; and/or (iii). selecting a fraction of the cell population that is CD34 positive and/or CD43 negative and wherein the sorting and cell fraction selection is performed on a day selected from about day 6 to day 11 of differentiation (the contents of U.S. Pat. No. 9,834,754 are herein incorporated by reference in its entirety).

In some embodiments, thymic cells and/or effector cells can be cultured in the presence of extracellular vesicles (e.g., exosomes) derived from thymic cells and/or effector cells. Methods of preparing exosomes of thymic cells are described in US Patent Publication US2020299641 and (the contents of which are herein incorporated by reference in its entirety). In some embodiments, the exosomes can be derived from thymic cells engineered to ectopically express DLL1.

In some embodiments, effector cells such as T cells, can be derived by differentiation of other cell types. T cell differentiation can include four stages: 1) Mesoderm induction (at about days 1-4), 2) hematopoietic specification (at about days 4-8) 3) hematopoietic commitment and expansion (at about days 8-10), and/or 4) T-lymphoid differentiation. PSCs (iPSCs or ESCs) can be utilized as the starting cell population for mesoderm differentiation. These cells can be differentiated into mesoderm cells. The mesoderm cells can further be differentiated into hematopoietic cells which can be expanded in cell numbers. The cell culture systems for use in the present disclosure include, but are not limited to, a first cell culture media for mesoderm induction, a second cell culture media for hematopoietic specification and expansion, and a third cell culture media for T-lymphoid differentiation. The first cell culture media can include BMP4 (e.g., human BMP4) and bFGF (e.g., human bFGF). PSCs or ESCs can be used as the starting cell population. Undifferentiated PSCs or ESCs can be transferred to low-attachment plates to allow for the formation of embryoid bodies (EBs). The formation of EBs during the first stage can be facilitated by an overnight incubation in the presence of hBMP4. EBs can then be cultured with BMP4 and bFGF until day 4 to allow for mesoderm induction. The successful induction of mesoderm can be tested by, e.g., by measuring the percentage of KDR+PDGFR− cells. The second cell culture media can include VEGF (e.g., hVEGF), and a cocktail of hematopoietic cytokines. The cocktail of hematopoietic cytokines can include SCF (e.g., hSCF), Flt3L (e.g., hFlt3L), at least one cytokine, and bFGF for hematopoietic specification. The cytokine can be a Th1 cytokine, which includes, but is not limited to IL3, IL15, IL7, IL12 and IL21. EBs can be cultured in the second cell culture media for hematopoietic specification until about day 10. The EBs can be immunophenotypically analyzed by FACS for expression of CD34, CD31, CD43, CD45, CD41a, c-kit, Notch1, IL7Ra. In some embodiments, CD34+ cells from about day EBs express the highest levels of key transcription factors for lymphoid differentiation, e.g., CD127 (IL7Ra) and NotchL. The third cell culture media can include a feeder cell and SCF, Flt3L and at least one cytokine. The cytokine can be a Th1 cytokine, which includes, but is not limited to, IL3, IL15, IL7, IL12 and IL21. In some embodiments, at about day 10, the EBs can be dissociated and the hematopoietic precursors can be transferred onto a feeder cell to induce T-lymphoid differentiation in an established co-culture system in the presence of the SCF, Flt3L and Th1 cytokine(s) (e.g., IL-7). In some embodiments, co-culture system can include thymic cells and/or feeder cultures, e.g., OP9-DL11 feeder cells.

In some embodiments, co-culture can be performed using a co-culture medium. In some embodiments, the co-culture medium can include StemSpan SFEM II and StemSpan™ T Cell Progenitor Maturation Supplement. In some embodiments, the co-culture medium can include αMEM, 4% B27 supplement, 30 uM Ascorbic acid, 50 ng/ml IL7, 50 ng/ml FLT3L, 50 ng/ml TPO, 50 ng/ml SCF, and/or 1× Pen Strep. In some embodiments, the co-culture medium can include DMEM/F12, 1% B27 supplement without vitamin A, 50 μM Ascorbic acid, 50 ng/ml FGF8b, 50 ng/ml BMP, 50 ng/ml FGF10, 2 uM CHIR99021, 0.1% ITS, 0.0025% KSR, 0.5× Pen Strep, 1× NEAA, 1% N2, 1% Glutamax, 1% β-ME, 50 ng/ml IL7, 50 ng/ml FLT3L, 50 ng/ml TPO, and/or 50 ng/ml SCF.

### Aggregate Size

In some embodiments, cells of the disclosure can be cultured in 3-dimensional culture. In some embodiments, cells of the disclosure can be in the form of aggregates or spheroids. The term “spheroid” refers to clusters of cells and/or cell colonies. Spheroids can be formed from various cell types, for example, thymic cells, pluripotent cells, effector cells, stem cells, and/or supporting cells. Spheroids can have sphere-like or irregular shapes. Spheroids can contain heterogeneous populations of cells, cell types, cells of different states, such as proliferating cells, quiescent cells, and necrotic cells.

In some embodiments, spheroid/aggregate size can be tuned. For example, aggregate size in pluripotent stem cells can be critical during expansion period since the size of the aggregate can determine oxygen distribution within the cell spheroid resulting in discrete zones composed of outside, middle, and inside spheroid regions along the oxygen supply from high to low, exhibiting proliferating, quiescent viable and apoptotic core property, respectively (Langan et al. Plos One. 2016; 11(2); the contents of which are herein incorporated by reference in its entirety). In some embodiments, the aggregates can be from about 50 μm to 500 μm, from about 100 μm to 1000 μm, from about 200 μm to 2000 μm, from about 250 μm to 2500 μm, from about 300 μm to 3000 μm, from about 400 μm to 4000 μm. In embodiment, the spheroid/aggregate size can be 250 μm.

### Methods of Use

The present disclosure provides methods of treating or preventing a condition in a subject. The methods can include administering to a subject, any of the populations of cells described herein, or a pharmaceutical composition comprising any of the populations of cells described herein, in an amount effective to treat or prevent the condition in the subject. The condition can be a cancer, an immunodeficiency, an autoimmune condition, an infection, or a blood condition. The condition can be associated with the absence, decline or aberrant functioning of the thymus of the subject. For example, the condition can be Di George syndrome, thymoma (such as type A thymoma or type B thymoma), CHARGE syndrome, FOXN1 deficiency, PAX1 deficiency, TBX1 deficiency, thymus cancer, thymic atrophy (such as age-related thymic atrophy), thymic cyst, thymic hyperplasia, thymic hypoplasia, thymic aplasia, thymic dysplasia, thymic irradiation, myasthenia gravis, thymic carcinoma, thymic hyperplasia, thymic irradiation, or age- or infection-associated decline in thymic function.

In certain embodiments, the cells described herein can be used to treat a subject who can undergo a thymectomy surgery. In certain embodiments, the subject can have a congenital heart defect and can have received or is receiving an open-heart surgery. A subject can undergo thymectomy for the treatment of one or more indications associated with the thymus e.g., myasthenia gravis or thymoma.

Various cancers can be treated with pharmaceutical compositions of the present disclosure. Cancers can be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Cells, compositions and pharmaceutical compositions of the disclosure can be used to treat infectious diseases. Infectious disease can be caused by an organism such as, but are not limited to bacteria, viruses, protozoan, and/or fungi.

### IV. Pharmaceutical Compositions

Pharmaceutical compositions of the present disclosure can include compositions with one or more cells described herein, and one or more pharmaceutically or physiologically acceptable carriers, diluents or excipients. Such compositions can include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect prepared for intravenous administration.

In some embodiments, pharmaceutical formulations can include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier can be supplemented with human serum albumin.

In some embodiment, the pharmaceutical composition can be substantially free of, e.g., there are no detectable levels of a contaminant, e.g., selected from endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus.

### Buffers

In some embodiments, pharmaceutical compositions of the present disclosure are prepared with one or more buffering agents.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Non-limiting examples include aqueous formulations such as pH 7.4 phosphate-buffered formulation, or pH 6.2 citrate-buffered formulation; formulations for lyophilization such as pH 6.2 citrate-buffered formulation with 3% mannitol, pH 6.2 citrate-buffered formulation with 4% mannitol/1% sucrose; or a formulation prepared by the process disclosed in U.S. Pat. No. 8,883,737 to Reddy et al., the contents of which are incorporated herein by reference in their entirety.

In some embodiments, pharmaceutical compositions of the present disclosure are formulated in parenteral dosage forms. The parenteral formulations can be aqueous solutions containing carriers or excipients such as salts, carbohydrates and buffering agents (e.g., at a pH of from 3 to 9), or sterile non-aqueous solutions, or dried forms which can be used in conjunction with a suitable vehicle such as sterile, pyrogen-free water. For example, an aqueous solution of the therapeutic agents of the present disclosure comprises an isotonic saline, 5% glucose or other pharmaceutically acceptable liquid carriers such as liquid alcohols, glycols, esters, and amides, for example, as disclosed in U.S. Pat. No. 7,910,594 to Vlahov et al. (Endocyte), the contents of which are incorporated herein by reference in their entirety. In another example, an aqueous solution of the therapeutic agents of the present disclosure comprises a phosphate buffered formulation (pH 7.4) for intravenous administration as disclosed in Example 23 of WO2011014821 to Leamon et al., the contents of which are incorporated herein by reference in their entirety. The parenteral dosage form can be in the form of a reconstitutable lyophilizate comprising the dose of the therapeutic agents of the present disclosure. Any prolonged release dosage forms known in the art can be utilized such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

### Nutrients

In some embodiments, the pharmaceutical compositions of the present disclosure include one or more nutrients that promote the health, survival, and/or proliferation of the cells described herein.

In some embodiments, the pharmaceutical formulations include vitamins. In some embodiments, the pharmaceutical compositions include 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B 12, or the pharmaceutical compositions includes combinations thereof or salts thereof. In some embodiments, the pharmaceutical compositions include or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B 12. In some embodiments, the vitamins include or consist essentially of biotin, DL alpha tocopherol acetate, DL alpha-tocopherol, vitamin A, or combinations or salts thereof.

In some embodiments, the pharmaceutical compositions further include proteins. In some embodiments, the proteins include albumin or bovine serum albumin, a fraction of BSA, catalase, insulin, transferrin, superoxide dismutase, or combinations thereof. In some embodiments, the pharmaceutical compositions include one or more of the following: corticosterone, D Galactose, ethanolamine, glutathione, L-carnitine, linoleic acid, linolenic acid, progesterone, putrescine, sodium selenite, or triodo-I-thyronine, or combinations thereof.

In some embodiments, the pharmaceutical compositions include amino acids, inorganic ions, and/or monosaccharides. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions include sodium, potassium, calcium, magnesium, nitrogen, or phosphorus, or combinations or salts thereof. In some embodiments, the pharmaceutical compositions further include one or more of the following: molybdenum, vanadium, iron, zinc, selenium, copper, or manganese, or combinations In some embodiments, the pharmaceutical compositions further include one or more of the following: corticosterone, D-Galactose, ethanolamine, glutathione, L-carnitine, linoleic acid, linolenic acid, progesterone, putrescine, sodium selenite, or triodo-I-thyronine, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese.

### Preservatives

Exemplary preservatives can include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include, ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN® II, NEOLONE™ KATHON™, and/or EUXYL®.

### V. Dosing and Administration

The cells and pharmaceutical compositions of the present disclosure described above can be administered by any delivery route, systemic delivery or local delivery, which results in a therapeutically effective outcome.

In some embodiments, thymic cells and effector cells can be co-delivered to the same anatomic location in a subject. In some embodiments, thymic cells and effector cells can be delivered to the different anatomic locations in a subject.

In some embodiments, thymic cells and effector cells can be delivered to the subject at the same time through the same delivery route or through a different delivery route.

In some embodiments, thymic cells can be administered to the subject prior to the administration of effector cells.

In some embodiments, thymic cells can be administered to the subject after the administration of effector cells.

Non-limiting examples of delivery routes include, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, pharmaceutical compositions containing cells of the disclosure can be delivered intrathymically (into the thymus).

In some embodiments, the pharmaceutical compositions containing cells of the disclosure can be surgically placed in the subject. As non-limiting examples, the cells can be surgically placed in the kidney capsule or in the quadriceps muscles in the thigh.

In some embodiments, cells and pharmaceutical compositions can be administered intra hepatically, via intrasplenic injection or via intraportal injection.

Cells and pharmaceutical compositions described herein can be provided to the subject by directly injection into the marrow of the bone (herein referred to as intraosseous infusion). The bone can be a long bone such as the tibia, fibula, femur, metatarsals, phalanges of the lower limbs, the humerus, radius, ulna, metacarpals, and/or phalanges of the upper limb.

### Parenteral and Injectable Administration

In some embodiments, the cells and pharmaceutical compositions described herein can be administered parenterally.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This can be accomplished by the use of liquid suspensions of crystalline or amorphous material with poor water solubility. The rate of absorption of active ingredients depends upon the rate of dissolution which, in turn, can depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Lymph Node Administration

In certain embodiments, the thymic cells and/or the pharmaceutical compositions of the present disclosure can be delivered into the subject for engraftment into the lymph node. In some embodiments, the thymic cells and/or the pharmaceutical compositions of the present disclosure can be delivered into the subject in an amount effective to form an ectopic thymus tissue in the lymph node. In certain embodiments, the methods and compositions described herein are used to deliver cells and/or pharmaceutical compositions of the disclosure into a lymph node of the subject, allowing the thymic cells to engraft and produce an ectopic thymus in the lymph node. In certain embodiments, the ectopic thymus can restore the thymic function of the subject, e.g., supplements or augments one or more functions that a normal healthy thymus organ can perform. For example, but not by way of limitation, the ectopic thymus can participate in immunomodulation of the body by promoting in T cell growth, development, maturation, selection and/or function.

Non-limiting examples of lymph nodes to which the cells and pharmaceutical compositions can be delivered include jejunal, popliteal, axillary, periportal lymph node, abdominal lymph nodes, celiac lymph nodes, paraaortic lymph nodes, splenic hilar lymph nodes, porta hepatis lymph nodes, left gastric lymph nodes, right gastric lymph nodes, left gastroomental (gastroepiploic) lymph nodes, right gastroomental (gastroepiploic) lymph nodes, retroperitoneal lymph nodes, pyloric lymph nodes (e.g., supra pyloric lymph nodes, sub pyloric lymph nodes, retro pyloric lymph nodes), pancreatic lymph nodes (e.g., superior pancreatic lymph nodes, inferior pancreatic lymph nodes, splenic lineal lymph nodes lymph nodes), splenic lymph nodes, hepatic lymph nodes (e.g., cystic lymph nodes, foraminal lymph nodes, foramen of Winslow), pancreaticoduodenal lymph nodes (e.g., superior pancreaticoduodenal lymph nodes, inferior pancreaticoduodenal lymph nodes), superior mesenteric lymph nodes, ileocolic lymph nodes, prececal lymph nodes, retrocecal lymph nodes, appendicular lymph nodes, mesocolic lymph nodes (e.g., paracolic lymph nodes, left colic lymph nodes, middle colic lymph nodes, right colic lymph nodes, inferior mesenteric lymph nodes, sigmoid lymph nodes, superior rectal lymph nodes), common iliac lymph nodes (e.g., medial common iliac lymph nodes, intermediate common iliac lymph nodes, lateral common iliac lymph nodes, subaortic common iliac lymph nodes, common iliac nodes of promontory), and external iliac lymph nodes (e.g., medial external iliac lymph nodes, intermediate external iliac lymph nodes, lateral external iliac lymph nodes, medial lacunar-femoral lymph nodes, intermediate lacunar-femoral lymph nodes, lateral lacunar-femoral lymph nodes, interiliac external iliac lymph nodes, obturator-external iliac obturatory lymph nodes).

As a non-limiting example, any of the methods for transplantation of thymic tissue into the lymph node described in International Patent Publication WO2021026195 can be useful in the present disclosure (the contents of which are herein incorporated by reference in its entirety).

### Depot Administration

As described herein, in some embodiments, cells and compositions including pharmaceutical compositions of the present disclosure are formulated in depots for extended release. Generally, specific organs or tissues (“target tissues”) are targeted for administration. In some embodiments, localized release is affected via utilization of a biocompatible device. For example, the biocompatible device can restrict diffusion of the cells in the subject.

In some aspects of the present disclosure, cells, compositions and pharmaceutical compositions are spatially retained within or proximal to target tissues. Provided are methods of providing pharmaceutical compositions, to target tissues of mammalian subjects by contacting target tissues (which include one or more target cells) with pharmaceutical compositions, under conditions such that they are substantially retained in target tissues, meaning that at least 10, 20, 30, 40, 50, 60, 70, 80, 85, 90, 95, 96, 97, 98, 99, 99.9, 99.99, or greater than 99.99% of the composition is retained in the target tissues. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions administered to subjects are present at a period of time following administration.

### Dose and Regimen

The present disclosure provides methods of administering cells, compositions and pharmaceutical compositions in accordance with the present disclosure to a subject in need thereof. The pharmaceutical compositions including the cells described can be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject can be a human, a mammal, or an animal. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, and the route of administration.

In some embodiments, a dose of cell, compositions and/or pharmaceutical compositions described herein can be about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of cell, compositions and/or pharmaceutical compositions described herein can be at least about 1×10⁶, 2×10⁶, 3×10⁶, 5×10⁶, 1×10⁷, 2×10⁷, 5×10⁷, 1×10⁸, 2×10⁸, 3×10⁸, or 5×10⁸ cells/kg. In some embodiments, a dose of cell, compositions and/or pharmaceutical compositions described herein can be up to about 1×10⁶, 2×10⁶, 3.6×10⁶, 5×10⁶, 1×10⁷, 2×10⁷, 5×10⁷, 1×10⁸, 2×10⁸, 3×10⁸, or 5×10⁸ cells/kg. In some embodiments, a dose of cell, compositions and/or pharmaceutical compositions described herein can be about 1×10⁷, 2×10⁷, 5×10⁷, 1×10⁸, 2×10⁸, 3×10⁸, 5×10⁸, 1×10⁹, 2×10⁹, or 5×10⁹ cells/kg. In some embodiments, a dose of cell, compositions and/or pharmaceutical compositions described herein can be about 1×10⁷, 2×10⁷, 5×10⁷, 1×10⁸, 2×10⁸, 3×10⁸, 5×10⁸, 1×10⁹, 2×10⁹, or 5×10⁹ cells/kg. In some embodiments, a dose of cell, compositions and/or pharmaceutical compositions described herein can be about 1×10⁷, 2×10⁷, 5×10⁷, 1×10⁸, 2×10⁸, 3×10⁸, 5×10⁸, 1×10⁹, 2×10⁹, or 5×10⁹ cells/kg. In some embodiments, a dose of cell, compositions and/or pharmaceutical compositions described herein can be about 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells/kg. In some embodiments, a dose of cell, compositions and/or pharmaceutical compositions described herein can be about 1-3×10⁷ to 1-3×10⁸ cells/kg.

In certain embodiments, the cell described herein or pharmaceutical compositions in accordance with the present disclosure can be administered at about 10 to 600 μl/site, 50 to about 500 μl/site, 100 to about 400 μl/site, 120 to about 300 μl/site, 140 to about 200 μl/site, about 160 μl/site.

The desired dosage can be delivered at least once, three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

The desired dosage of the cells of the present disclosure can be administered one time or multiple times. The cells, compositions and pharmaceutical formulations can be administered regularly with a set frequency over a period of time, or continuously as a “continuous flow”. A total daily dose, an amount given or prescribed in 24-hour period, can be administered by any of these methods, or as a combination of these methods.

In some embodiments, delivery of the cells to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

The cells of the present disclosure can be used in combination with one or more other therapeutic, prophylactic, research or diagnostic agents, or medical procedures, either sequentially or concurrently. In general, each agent will be administered at a dose and/or on a time schedule determined for that agent. In some embodiments, the present disclosure encompasses the delivery of pharmaceutical, prophylactic, research, or diagnostic compositions in combination with agents that can improve their bioavailability, reduce and/or modify their metabolism, inhibit their excretion, and/or modify their distribution within the body.

For example, the cells of the present disclosure are administered as a biocompatible device that restricts diffusion in the subject to increase bioavailability in the area targeted for treatment. The cells of the present disclosure can also be administered by local delivery.

The term “conditioning regime” refers to a course of therapy that a patient undergoes before stem cell transplantation. For example, before hematopoietic stem cell transplantation, a patient can undergo myeloablative therapy, non-myeloablative therapy or reduced intensity conditioning to prevent rejection of the stem cell transplant even if the stem cell originated from the same patient. The conditioning regime can involve administration of cytotoxic agents. The conditioning regime can also include immunosuppression, antibodies, and irradiation. Other possible conditioning regiments include antibody mediated conditioning (see e.g., Czechowicz et al., 318 (5854) Science 1296-9 (2007); Palchaudari et al., 34(7) Nature Biotechnology 738-745 (2016); Chhabra et al., 10:8(351) Science Translational Medicine 351ra105 (2016)) and CAR-T mediated conditioning (see, e.g., Arai et al., 26(5) Molecular Therapy 1181-1197 (2018); each of which is hereby incorporated by reference in its entirety). The conditioning regimen is also designed to create niche “space” to allow the transplanted cells to have a place in the body to engraft and proliferate. In hematopoietic stem cell transplantation, for example, the conditioning regimen creates niche space in the bone marrow for the transplanted hematopoietic stem cells to engraft into. Without a conditioning regimen the transplanted hematopoietic stem cells cannot engraft. In some embodiments, a subject can be dosed with cells, compositions and/or pharmaceutical formulation of the present disclosure following treatment with a conditioning regime.

### VI. Definitions

Expression: As used herein, “expression” and grammatical equivalents thereof, in the context of a marker, refers to production of the marker as well as level or amount of the marker. For example, expression of a marker or presence of a marker in a cell or a cell is positive for a marker, refers to expression of the marker at a level that is similar to a positive control level. The positive control level can be determined by the level of the marker expressed by a cell known to have the cell fate associated with the marker. Similarly, absence of expression of a marker or a cell is negative for a marker, refers to expression of the marker at a level that is similar to a negative control level. The negative control level can be determined by the level of the marker expressed by a cell known to not have the cell fate associated with the marker. As such, absence of a marker does not simply imply an undetectable level of expression of the marker, in certain cases, a cell can express the marker but the expression can be low compared to a positive control or can be at a level similar to that of a negative control.

Effector cell: As used herein, an “effector cell” refers to any cell or cell type which, when in contact with or in proximity to a thymic cell, acquires the ability to execute, initiate or propagate a signal or a cell death trigger. “Contact or proximity” refers to spatiotemporal closeness sufficient to enable cell-intrinsic or cell-extrinsic (e.g., cell-to-cell) signaling or other communication or interaction.

Lymphocyte: As used herein, a “lymphocyte” embraces the meanings and uses that a person of ordinary skill in the art would understand the term to embrace, and additionally refers to a type of immune cell originating in the bone marrow that resides in lymphoid tissues or blood. In some embodiments, lymphocytes undergo maturation in the thymus.

Negative: As used herein, the term “negative” (which can be abbreviated as“−”), as used herein with reference to expression of the indicated cell marker, means that the cell does not express the indicated cell marker at a detectable level.

Positive: As used herein, the term “positive” (which can be abbreviated as “+”), with reference to expression of the indicated cell marker, means that the cell expresses the indicated cell marker at any detectable level, which can include, for example, expression at a low (but detectable) level as well as expression at a high (hi) level.

Pre-T cell: As used herein, a “pre-T cell” refers to a lymphocyte that is capable of maturing or differentiating into a T cell.

Soluble factor: As used herein, a “soluble factor” refers to any protein or peptide that can bind to a cell surface molecule or be taken up by a cell. Uptake by the cell can be by passive diffusion, by a transporter, and/or by endocytosis.

Thymic cell or origin or lineage: As used herein, “thymic cell or thymic origin or thymic lineage” refers to a cell with one or more phenotypic or genotypic markers associated with a cell derived from the thymus or a cell destined to become a cell of the thymus. As used herein, the thymus can be an embryonic, a fetal or an adult thymus.

Variant: The term “variant” as used in reference to a biomolecule (e.g., a training factor or a terminal factor) refers to a biomolecule that is related to or derived from a parent molecule. The variant can be, for example, a modified form, a truncated form, a mutated form, a homologous form, or other altered form of the parent molecule. The term variant can be used to describe either polynucleotides or polypeptides.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### EXAMPLES

### Example 1. Preparation of Pluripotent Stem Cells

On day 1, 2.5 million single iPSCs were placed in suspension on day 1 and ROCK inhibitor Y27632(10 μM) was added. The cells were placed on a shaker at 70 RPM. On day 2-3, the differentiation was continued, and half of the media was changed with fresh media daily on day 2 and 3. At this time, the spheroids were about 250 μm in diameter. The cells were placed on a shaker at 70 RPM. On day 4, the supernatant was transferred alongside spheroids into 15-ml conical tubes. The spheroids were centrifuged at 250 g for 5 min at room temperature and the supernatant was aspirated.

The spheroids were washed with PBS, centrifuged at 250 g for 5 min and the PBS was aspirated (in the hood). 3-4 ml of pre-warmed Accutase was added into a 15 ml conical tube. The tube was tapped to agitate the aggregates and the tube was placed inside the incubator for 5-7 minutes. The spheroids were agitated inside the 15 ml tube every 2-3 minutes. After 5 minutes, a 1000 μl pipette was used to gently agitate the aggregates in order to make turn spheroids into single cells. 7-8 ml of DMEM-F12 media was added to each 15-ml tube and the spheroids were centrifuged at 250 g for 5 min at room temp and the supernatant was aspirated.

### Example 2. Generation of Definitive Endoderm from Pluripotent Stem Cells

On day 2 iPSCs are divided into 4 different groups and are tested for different conditions. Table 1 and Table 2 provide the culture media utilized and Table 3 provides the different culture conditions tested.

TABLE 1
Media A

| Component | Concentration | |
|---|---|---|
| DMEM-F12 | Not applicable | |
| Activin A | 100 | ng/mL |
| CHIR99021 | 2 | μM |

| | |
|---|---|
| ITS | 1:1000 |
| KSR | 0.05% |
| Pen Strep | 100X |

TABLE 2
Media B

| Component | Concentration | |
|---|---|---|
| DMEM-F12 | Not applicable | |
| Activin A | 100 | ng/mL |
| bFGF | 10 | ng/ml |
| LDN193189 (BMP | 200 | nM |

| | |
|---|---|
| inhibitor) | |
| ITS | 1:1000 |
| KSR | 0.05% |
| Pen Strep | 100X |

TABLE 3
Protocol optimization

| DAYS | 1 | 2 | 3 | 4 | 5 |
|---|---|---|---|---|---|
| Group 1 | Media A | Media B | Media B | — | |
| (G1) | | | | | |
| Group 2 | Media A + | Media B | Media B | — | |
| (G2) | PI-103 | | | | |
| | (50 nM) | | | | |
| Group 3 | Media A | Media A | Media B | Media B | Media B |
| (G3) | | | | | |
| Group 4 | Media A + | Media A + | Media B | Media B | Media B |
| (G4) | PI-103 | PI-103 | | | |
| | (50 nM) | (25 nM) | | | |

### Example 3. Generation of Thymic Cells from Pluripotent Stem Cells

Differentiation of iPSCs into Definitive Endoderm (DE)

Differentiation of iPSCs into DE requires proper timing for introducing small molecules and growth factors. Differentiation was performed as described in Example 2 or as follows. On Day 1, PSCs in spheroids were cultured in Media A/A 50%/50% (Stem Scale and DMEM-F12) containing activin A (100 ng/mL), CHIR99021 2 μM, KSR (0.05%), Pen strep (100×), PI-103 (25 nM). On day 2 and day3, cells were cultured in DMEM-F12 containing Activin A (100 ng/mL), CHIR99021 2 μM, ITS (1:1000), KSR (0.05%), Pen strep (100×), PI-103 (25 nM), non-essential amino acids (NEAA) (100×). On day 4-5, cells were cultured in DMEM-F12 containing Activin A (100 ng/mL), LDN193189 (100 nM), Insulin-Transferrin-Selenium (ITS-G) (1:1000), KSR (0.05%), Pen strep (100×), NEAA (100×).

Differentiation of definitive endoderm (DE) into anterior foregut endoderm (AFE) was performed as follows. On days 6-7, cells were cultured in AFE media prepared by adding SB431542 (10 μM), LDN193189 (0.1 μM), Insulin-Transferrin-Selenium (ITS-G) (1:1000), KSR (0.05%), Penicillin-streptomycin (100×), NEAA (100×) to DMEM-F12.

Differentiation of anterior foregut endoderm (AFE) to ventral pharyngeal endoderm (VPE) was performed as follows: From days 8-13, cells were cultured in VPE media prepared by adding retinoic acid (0.1 μM), FGF8b (50 ng/ml), SHH (SAG) (100 ng/ml), EGF (50 ng/ml), Insulin-Transferrin-Selenium (ITS-G) (1:1000), KSR (0.05%), Penicillin-streptomycin (100×) and NEAA (100×) to DMEM-F12.

Differentiation of Ventral Pharyngeal Endoderm (VPE) to Thymic Epithelial Progenitors was performed using one of two protocols. In protocol 1, from days 14-16 were cultured in DMEM-F12 containing BMP4 (50 ng/ml), FGF8b (50 ng/ml), EGF (50 ng/ml), SANT-1 (0.25 μM), CHIR99021 (2 μM), Ascorbic Acid (30 μM), Insulin-Transferrin-Selenium (ITS-G) (1:1000), KSR (0.05%), Pen strep (100×), NEAA (100×). In a second protocol, on day 14-16, cells were cultured in DMEM F12 containing Noggin (50 ng/ml), FGF8b (50 ng/ml), EGF (50 ng/ml), CHIR99021 (2 μM), Ascorbic Acid (30 μM), Insulin-Transferrin-Selenium (ITS-G) (1:1000), KSR (0.05%), Pen strep (100×), NEAA (100×). From day 17-21, cells were cultured in DMEM-F12 containing BMP4 (50 ng/ml), FGF8b (50 ng/ml), EGF (50 ng/ml), SANT-1 (0.25 μM), CHIR99021 (2 μM), Ascorbic Acid (30 μM), Insulin-Transferrin-Selenium (ITS-G) (1:1000), KSR (0.05%), Pen strep (100×), NEAA (100×).

Complete Media was changed every day. The cells were collected and allowed to settle in the laminar hood. The supernatant was aspirated, and freshly prepared media was added. Additionally, cells were washed with DMEM-F12 and allowed to settle for 5 min, between differentiation stages.

### Example 4. 3D Suspension Culture of Cell Lines

Three pluripotent cell lines were established for use in optimizing differentiation protocols namely, a human ES FOXN1-GFP reporter line: a human iPSC line (A15849 GIBCO) and a research grade iPSC line from GMP-grade therapeutic iPSC cell line. All three cell lines were adapted for in vitro 3D culture using Matrigel. Pluripotency of the lines was analyzed by flow cytometry analysis for expression of stem cell markers TRA-1-60 and SSEA-4. Over 95% of the pluripotent stem cells were found to express TRA-1 and SSEA-4.

### Example 5. Characterization of DE Cells

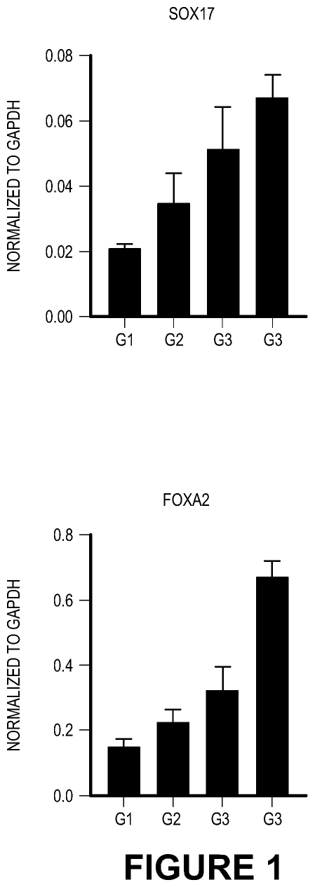

The definitive endodermal (DE) cells obtained from the four different culture conditions described in Example 2 as G1 to G4 were analyzed for markers of DE cells namely, SOX17 and FOXA2. Culture Group1 (G1) to 4 (G4) showed a relative increase in the of expression level of the key biomarkers of DE (SOX17 and FoxA2) (see FIG. 1).

Cells were also positive for expression of markers associated with DE cells namely EPCAM and SOX17. The number of double positive cells for G1, G2, G3 and G4 were 18.2%, 16.2%, 54.3%, and 69.6% respectively. G4 produced the greatest number of positive cells in this study.

Manipulation of starting size of spheroids, increased the frequency of DE cells to greater than 95%.

### Example 6. Effect of Varying Growth Factors

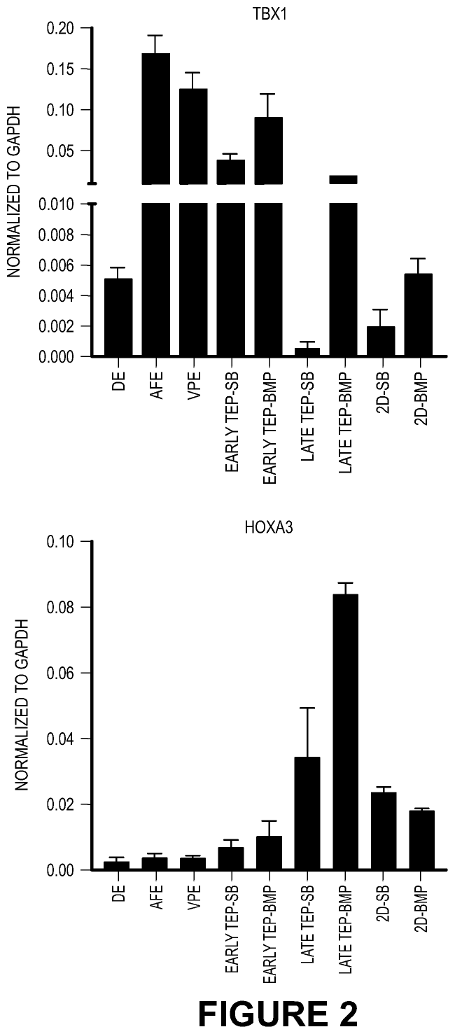

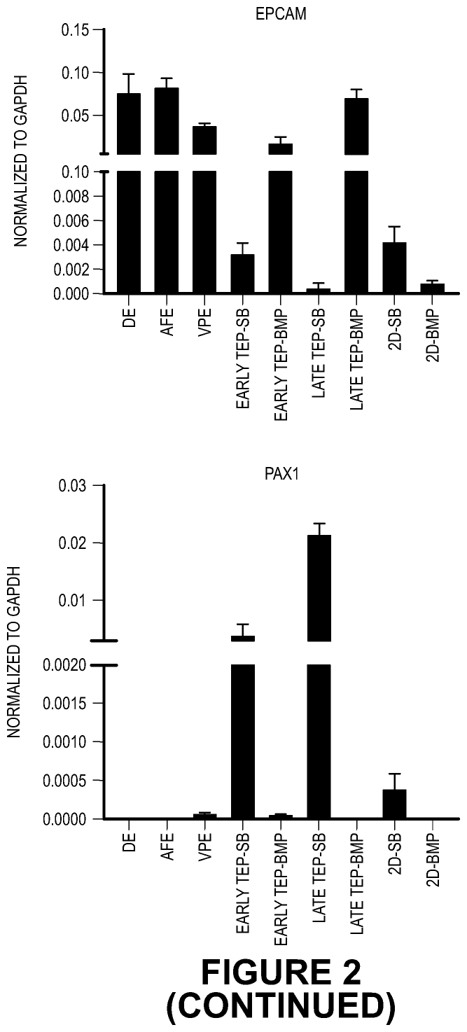

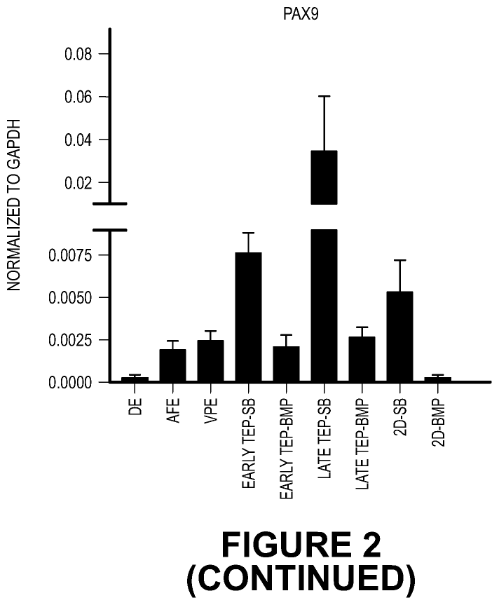

Differentiation of DE cells to thymic cells can be promoted by adding factors known to be important for DE to thymic differentiation. An example is BMP which is known to be crucial for the development and maturation of thymic progenitor cells from the third pharyngeal pouch. The impact of the timing of addition of BMP was explored (FIG. 2). Spheroids were induced to differentiate from iPSC state to TEP and timing of addition of BMP was varied. At different stages (DE, AFE, VPE, early and late TEP) spheroids were collected and analyzed for biomarkers related to the different stages. We compared condition with or without BMP at early and late TEP stages. BMP suppressed PAX1 and PAX9 but upregulated TBX1 and HOXA3.

### Example 7. Equivalence of In Vitro to In Vivo Transition

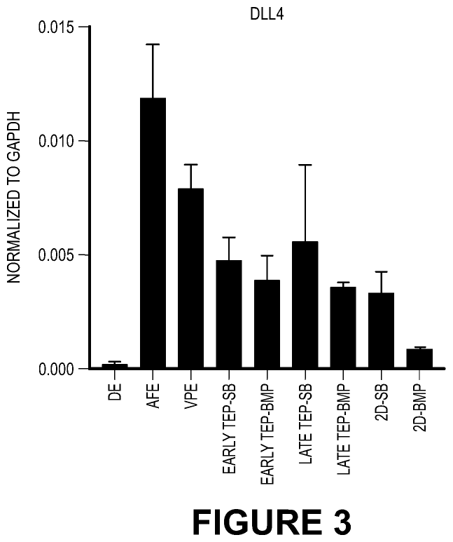

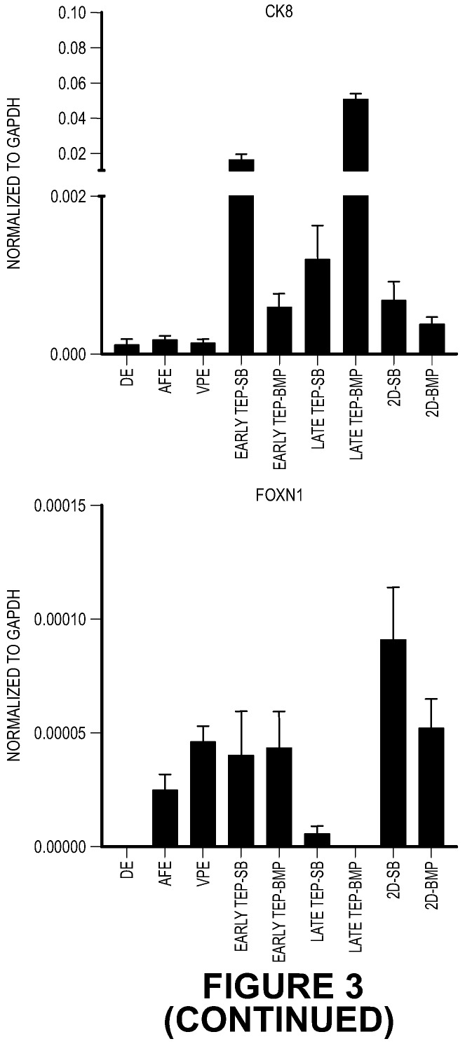

It is expected that thymic cells generated by the methods described herein, will, upon in vivo transplantation, differentiate into mTECs and cTECs. To test whether spheroids in 3D spread out into epithelial sheets in vivo, spheroids were allowed to adhere on Matrigel and to further differentiate in 2-D. After 4-5 days of culture on Matrigel, the expression of genes associated with TECs was examined. As shown in FIG. 3, DLL4 and CK8 expression was downregulated, whereas FOXN1 expression was upregulated. FOXN1 is a critical regulator of thymic differentiation.

### Example 8. Effect of Freeze and Thaw on TEP Phenotype

TEPs can need to be frozen, thawed and re-cultured for various clinical applications. Expression of markers associated with thymic cells was examined after freeze and thaw.

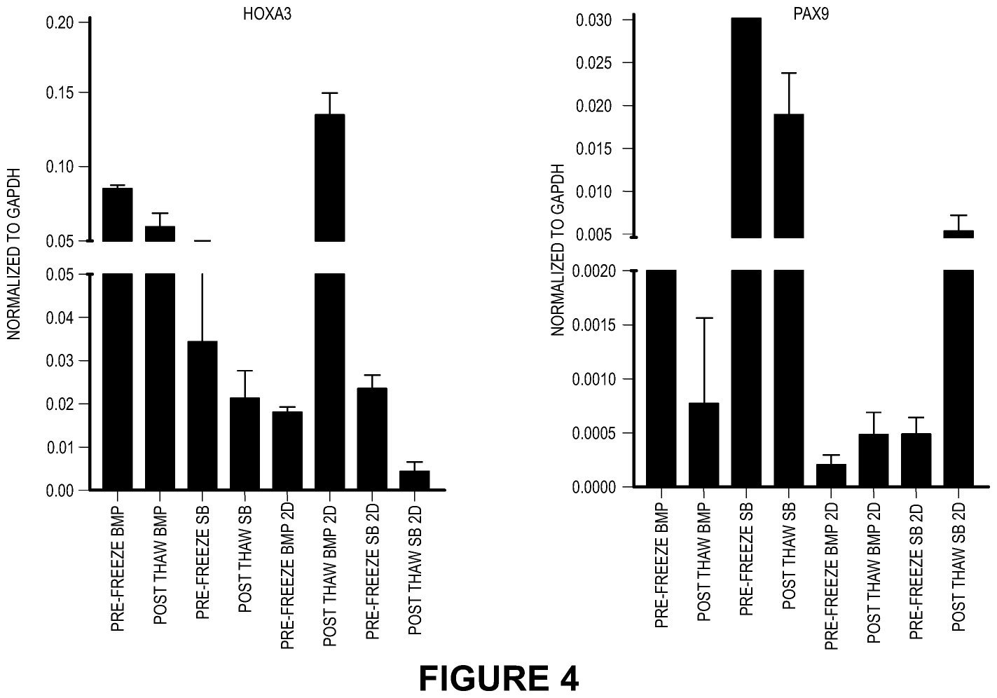

TEPs that were frozen, thawed and resuspended in 3D culture were able to reform spheroids in about 3-4 days. Expression of genes associated with late TEP stage was compared before freezing and after post-thaw spheroid formation. Expression levels of genes namely, HOXA3, PAX9, EPCAM, CXCL12 were similar before freezing and after thawing showing that TEPs can be frozen and thawed and re-cultured for transplantation (see FIG. 4).

### Example 9. Thymic Cell Biomarkers

Several putative markers have been associated with TEP cells. These include CD205 (Mohtashami M et al 2013 Int Immunol 25:601 and Campinoti S et al 2020 Nat Comm 11:1), EPCAM (Parent et al 2013 Cell Stem Cell 13:219), and Claudin 3 and 4 (Hamazaki Y et al 2016 Immunol Reviews 271:38).

Dual positive CD205+/EPCAM+ cells have been used to quantify progenitor cells in the thymus (Campinoti S et al 2020 Nat Comm 11:1). This combination of markers was used to characterize the phenotype TEPs.

Using CD205/EPCAM double staining to analyze TEPs at early and late stage of differentiation CD205+/EPCAM+ cells were found to increase in frequency from <1% to >25%. These data show that CD205+/EPCAM+ can be used as a marker for TEP cells.

### Example 10. In Vitro Assay for Differentiating T Cells

To compare and correlate in vitro function of iPSC-TEP with functional output observed after in vivo transplantation, an in vitro assay for differentiating T cells from CD34+HSC was developed. As a positive control, the StemSpan T-Cell kit (StemCell Technology) was used to generate double-negative (DN) Pro-T cells from CD34+HSCs are then matured into double-positive CD4 and CD8 T cells. At day 21, the percentage of CD4+/CD8+ cells was 5.3%, which increased to 49.4% at day 28, 53.2% at day 35, and 86.8% cells at day 42.

### Example 11. Preparation of Thymic Cells from Pluripotent Stem Cells

The protocol for preparation of thymic cells from pluripotent stem cells was optimized.

### Experiment 16A and 16B

In this experiment, the utility of adding Experiment 16 Medium A/A was added 2 days before the addition of Experiment 16 Medium A. Spheroids were seeded to Geltrex in 2D culture medium at DE induction stage in Experiment 16A whereas in Experiment 16B, a single cell suspension was prepared prior to seeding onto Geltrex.

On day 1, 2.5 million single iPSCs were placed in suspension on day 1 and ROCK inhibitor Y27632 (10 μM) was added. The shaker was set to 70 RPM. On day 2, the supernatant alongside spheroids was transferred into 15-ml conical tubes. The spheroids were centrifuged at 250 g for 5 minutes at room temperature and aspirate the supernatant. The spheroids were washed with PBS and centrifuged again. The supernatant was removed and 1 ml media was added to each well of a 6-well low adhesion plate. 6 ml of Experiment 16 Media A was added to one-day old iPSC aggregates and gently mixed before adding 1 ml to each well of a 6-well plate.

Differentiation of iPSCs into Definitive Endoderm (DE)

Differentiation of iPSCs into DE requires proper timing for introducing small molecules and growth factors. On day 1, cells were treated with Experiment 16 Media A/A comprising 50%/50% (Stem Scale and DMEM-F12), and with factors/small molecules Activin A (100 ng/mL), CHIR99021 (2 μM), KSR (0.05%), Penicillin streptomycin (100×), PI-103 (25 nM). On Day 2-3, Experiment 16 Media A comprising DMEM-F12, Activin A (100 ng/mL), CHIR99021 2 μM, ITS (1:1000), KSR (0.05%), Penicillin streptomycin (100×), PI-103 (25 nM), and NEAA (100×) was added. On day 4-5, Experiment 16 Media B comprising DMEM-F12, Activin A (100 ng/mL), LDN193189 (100 nM), Insulin-Transferrin-Selenium (ITS-G) (1:1000), KSR (0.05%), Penicillin streptomycin (100×) and NEAA (100×) was added.

Differentiation of Definitive Endoderm (DE) into Anterior Foregut Endoderm (AFE)

On day 6-7, Experiment 16 AFE Media DMEM-F12, and with factors/small molecules SB431542(10 μM), LDN193189 (0.1 μM), Insulin-Transferrin-Selenium (ITS-G) (1:1000), KSR (0.0025%), Penicillin streptomycin (100×), B27-without Vitamin A (VIT A) (0.5×), and NEAA (100×) was added. Day 7, only half the media was changed.

### Differentiation of Anterior Foregut Endoderm (AFE) to Ventral Pharyngeal Endoderm (VPE)

On day 8-9, Experiment 16 VPE1 media comprising, DMEM-F12, and with factors/small molecules SB431542 (10 uM/ml), Retinoic Acid (0.1 μM), FGF8b (50 ng/ml), Retinoic acid (0.1 μM), Insulin-Transferrin-Selenium (ITS-G) (1:1000), KSR (0.0025%), Penicillin streptomycin (100×), NEAA (100×) and B27-without VIT A (0.5×). On day 9, only half the media was changed. On day 10-11, VPE2 media containing DMEM-F12, Retinoic Acid (0.1 μM), FGF8b (50 ng/ml), Noggin (50 ng/ml), CHIR99021 (2 μM), Ascorbic Acid (30 μM), Insulin-Transferrin-Selenium (ITS-G) (1:1000), KSR (0.0025%), Penicillin streptomycin (100×), and NEAA (100×) B27-without VIT A (0.5×) was added. Day 11, only half the media was changed.

### Differentiation of Ventral Pharyngeal Endoderm (VPE) to Thymic Epithelial Progenitors (TEP)

On day 12-14, Experiment 16 TEP Media containing DMEM-F12, and with factors/small molecules FGF8b (50 ng/ml), BMP4 (50 ng/ml), CHIRR99 (2 μM), Ascorbic Acid (30 μM), Insulin-Transferrin-Selenium (ITS-G) (1:1000), KSR (0.0025%), Penicillin streptomycin (100×), NEAA (100×) was added. On day13 and 14, only half the media was changed. On day 14-15, TEC1 media containing DMEM-F12, BMP4 (50 ng/ml), FGF8b (50 ng/ml), Ascorbic Acid (30 μM), Retinoic Acid (0.1 μM), Insulin-Transferrin-Selenium (ITS-G) (1:1000), KSR (0.0025%), Pen strep (100×), and NEAA (100×), was added. On day16, cells were seeded on 24-well plate coated with Geltrex as aggregates (Experiment 16A) or single cell (Experiment 16B). Day 16-27, TEC2 Media containing DMEM-F12, BMP4 (50 ng/ml), FGF8b (50 ng/ml), Ascorbic Acid (30 μM), Retinoic Acid (0.1 μM), Insulin-Transferrin-Selenium (ITS-G) (1:1000), KSR (0.0025%), Pen strep (100×), NEAA (100×), B27-without VIT A (0.5×), N2 (100×), Glutamax (100×), and BME (100×) was added. All the media were changed daily.

No improvement in DE differentiation was observed. Seeding aggregate into 2D generated better growth of cells in 2D and more robust induction of differentiation.

### Experiment 18

In this experiment, transferring cells to 2D culture at VPE stage was tested.

Expansion of iPSC Line in Suspension Culture

On day 1, 2.5 million single iPSCs were placed in suspension on day 1 and ROCK inhibitor Y27632 (10 μM) was added. The shaker was set to 70 RPM. On day 2, the supernatant alongside spheroids was transferred into 15-ml conical tubes. The spheroids were centrifuged at 250 g for 5 minutes at room temperature and aspirate the supernatant. The spheroids were washed with PBS and centrifuged again. The supernatant was removed and 1 ml media was added to each well of a 6-well low adhesion plate. 6 ml of Experiment 18 Media A was added to one-day old iPSC aggregates and gently mixed before adding 1 ml to each well of a 6-well plate.

Differentiation of iPSCs into Definitive Endoderm (DE)

Differentiation of iPSCs into DE requires proper timing for introducing small molecules and growth factors. On day 1, Experiment 18 Media A/A comprising 50%/50% (Stem Scale and DMEM-F12) was added. On day 2-3, Experiment 18 Media A comprising DMEM-F12 and with factors/small molecules Activin A (100 ng/mL), CHIR99021 2 μM, ITS (1:1000), KSR (0.05%), Pen strep (100×), PI-103 (25 nM), NEAA (100×). On day 4-5, Experiment 18 Media B comprising DMEM-F12 with factors/small molecules Activin A (100 ng/mL), LDN193189 (100 nM), Insulin-Transferrin-Selenium (ITS-G) (1:1000), KSR (0.05%), Pen strep (100×), and NEAA (100×) was added.

Differentiation of Definitive Endoderm (DE) into Anterior Foregut Endoderm (AFE)

On day 6-7, Experiment 18 AFE Media comprising DMEM-F12 and with factors/small molecules SB431542(10 μM), LDN193189 (0.1 μM), Insulin-Transferrin-Selenium (ITS-G) (1:1000), KSR (0.0025%), Pen Strep (100×), B27-without VIT A (0.5×) and NEAA (100×) was added. On Day 7, only half the media was changed.

### Differentiation of Anterior Foregut Endoderm (AFE) to Ventral Pharyngeal Endoderm (VPE)

On day 8, the aggregates were seeded on Geltrex-coated 24-well plate. On day 8-9, the cells were transferred to Experiment 18 VPE1 Media comprising DMEM-F12 and with factors/small molecules namely, SB431542 (10 uM/ml), Retinoic Acid (0.1 μM), FGF8b (50 ng/ml), Retinoic acid (0.1 μM), Insulin-Transferrin-Selenium (ITS-G) (1:1000), KSR (0.0025%), Pen strep (100×), NEAA (100×), B27-without VIT A (0.5×) were added. On day 9, only half the media was changed.

On day 10-11, Experiment 18 VPE2 Media comprising DMEM-F12 and with factors/small molecules Retinoic Acid (0.1 μM), FGF8b (50 ng/ml), Noggin (50 ng/ml), CHIR99021 (2 μM), Ascorbic Acid (30 μM), Insulin-Transferrin-Selenium (ITS-G) (1:1000), KSR (0.0025%), Pen strep (100×), NEAA (100×), B27-without VIT A (0.5×) was added. On day 11, only half the media was changed.

### Differentiation of Ventral Pharyngeal Endoderm (VPE) to Thymic Epithelial Progenitors (TEP)

On day 12-14, Experiment 18 TEP Media containing DMEM-F12 and with factors/small molecules namely, FGF8b (50 ng/ml), BMP4 (50 ng/ml), CHIRR99 (2 μM), Ascorbic Acid (30 μM), Insulin-Transferrin-Selenium (ITS-G) (1:1000), KSR (0.0025%), Pen strep (100×), and NEAA (100×) was added. On Day13 and 14, only half the media was changed. On day 15-20, Experiment 18 TEC1 Media comprising DMEM-F12 and with factors/small molecules namely BMP4 (50 ng/ml), FGF8b (50 ng/ml), Ascorbic Acid (30 μM), Retinoic Acid (0.1 μM), Insulin-Transferrin-Selenium (ITS-G) (1:1000), KSR (0.0025%), Pen strep (100×) and NEAA (100×) was added.

On day21-22, Experiment 18 TEC2 Media containing DMEM-F12, with BMP4 (50 ng/ml), CHIRR9901 (2 μM), FGF8b (50 ng/ml), Ascorbic Acid (30 μM), Insulin-Transferrin-Selenium (ITS-G) (1:1000), KSR (0.0025%), Pen strep (100×), NEAA (100×), B27-without VIT A (0.5×), N2 (100×), Glutamax (100×) and BME (100×) was added. All the Media were changed daily.

TEPs generated in this experiment showed good FOXN1 expression.

### Experiment 21 and Experiment 22

In this experiment, the effect of adding PI-103 to Experiment 21/22 Medium B was tested. The effect of transitioning to 2D culture at the AFE stage was also tested.

Expansion of iPSC Line in Suspension Culture

On day 1, 2.5 million single iPSCs were placed in suspension on day 1 and ROCK inhibitor Y27632 (10 μM) was added. The shaker was set to 70 RPM. On day 2, the supernatant alongside spheroids was transferred into 15-ml conical tubes. The spheroids were centrifuged at 250 g for 5 minutes at room temperature and aspirate the supernatant. The spheroids were washed with PBS and centrifuged again. The supernatant was removed and 1 ml media was added to each well of a 6-well low adhesion plate. 6 ml of Experiment 21/22 Media A was added to one-day old iPSC aggregates and gently mixed before adding 1 ml to each well of a 6-well plate.

Differentiation of iPSCs into Definitive Endoderm (DE)

Differentiation of iPSCs into DE requires proper timing for introducing small molecules and growth factors. On Day 1-2, Experiment 21/22 Media A comprising DMEM-F12 and factors/small molecules namely Activin A (100 ng/mL), CHIR99021 2 μM, ITS (1:1000), KSR (0.05%), Pen strep (100×), PI-103 (25 nM) and NEAA (100×) was added. On day 3-5, Experiment 21/22 Media B comprising DMEM-F12 and factors/small molecules namely Activin A (100 ng/mL), LDN193189 (200 nM), PI-103 (25 nM), Insulin-Transferrin-Selenium (ITS-G) (1:1000), KSR (0.05%), Pen strep (100×) and NEAA (100×) was added. In Experiment 22, PI-103 (25 nM) was excluded.

Differentiation of Definitive Endoderm (DE) into Anterior Foregut Endoderm (AFE)

On day 6, the aggregates were seeded on Geltrex-coated 24-well plates. On day 6, the cells were culture in Experiment 21/22 AFE Media* comprising DMEM-F12 and factors/small molecules namely, SB431542 (10 μM), LDN193189 (200 nM), Ascorbic Acid (30 μM), Pen Strep (100×), N2 (100×), Glutamax (100×), BME (100×), and BSA (0.05%) were added.

### Differentiation of Anterior Foregut Endoderm (AFE) to Ventral Pharyngeal Endoderm (VPE)

On day 9-10, Experiment 21/22 VPE1 Media comprising DMEM-F12 and factors/small molecules namely SB431542 (10 uM/ml), Retinoic Acid (0.1 μM), FGF8b (50 ng/ml), Ascorbic Acid (30 μM), Insulin-Transferrin-Selenium (ITS-G) (1:1000), KSR (0.0025%), Pen strep (100×), NEAA (100×), B27-without VIT A (0.5×), Glutamax (100×), BME (100×) and N2(100×) was added. On day 10, only half the media was changed. On day 11-12, VPE2 Media containing, DMEM-F12 and factors/small molecules namely Retinoic Acid (0.1 μM), FGF8b (50 ng/ml), Noggin (50 ng/ml), CHIR99021 (2 μM), Ascorbic Acid (30 μM), Insulin-Transferrin-Selenium (ITS-G) (1:1000), KSR (0.0025%), Pen strep (100×), NEAA (100×), B27-without VIT A (0.5×), Glutamax (100×), BME (100×) and N2(100×) was added. On day 12, only half the media was changed.

### Differentiation of Ventral Pharyngeal Endoderm (VPE) to Thymic Epithelial Progenitors (TEP)

On day 12-14, Experiment 21/22 TEP Media containing, DMEM-F12 and factors/small molecules namely FGF8b (50 ng/ml), FGF10 (50 ng/ml), BMP4 (50 ng/ml), CHIRR99 (2 μM), Ascorbic Acid (30 μM), Insulin-Transferrin-Selenium (ITS-G) (1:1000), KSR (0.0025%), Pen strep (100×), NEAA (100×), B27-without VIT A (0.5×), Glutamax (100×), BME (100×) and N2(100×) were added. On day14 and 15, only half the media was changed.

Day16-18, Experiment 21/22 TEC Media containing DMEM-F12 and factors/small molecules namely BMP4 (50 ng/ml), FGF8b (50 ng/ml), Ascorbic Acid (30 μM), Retinoic Acid (0.1 μM), Insulin-Transferrin-Selenium (ITS-G) (1:1000), KSR (0.0025%), Pen strep (100×), NEAA (100×), B27-without VIT A (0.5×), N2 (100×), Glutamax (100×) and BME (100×) was added. All the Media were changed daily.

Addition of PI-103 to Experiment 21/22 Medium B enhanced DE differentiation. Transitioning to 2D culture at the AFE stage resulted in less FOXN1 expression compared to Experiment 18.

### Experiment 23

The effect of transitioning from 3D to 2D culture at VPE stage was tested. The impact of CHIR99021 addition to Experiment 23 Medium B was tested.

Expansion of iPSC Line in Suspension Culture

On day 1, 2.5 million single iPSCs were placed in suspension on day 1 and ROCK inhibitor Y27632 (10 μM) was added. The shaker was set to 70 RPM. On day 2, the supernatant alongside spheroids was transferred into 15-ml conical tubes. The spheroids were centrifuged at 250 g for 5 minutes at room temperature and aspirate the supernatant. The spheroids were washed with PBS and centrifuged again. The supernatant was removed, and 1 ml media was added to each well of a 6-well low adhesion plate. 6 ml of Experiment 23 Media A was added to one-day old iPSC aggregates and gently mixed before adding 1 ml to each well of a 6-well plate.

Differentiation of iPSCs into Definitive Endoderm (DE)

Differentiation of iPSCs into DE requires proper timing for introducing small molecules and growth factors. On day 1-2, cells were cultured in Experiment 23 Media A comprising DMEM-F12 and small molecules/factors namely Activin A (100 ng/mL), CHIR99021 2 μM, ITS (1:1000), KSR (0.05%), Pen strep (100×), PI-103 (25 nM), and NEAA (100×). On day 3-4, Experiment 23 Media B containing DMEM-F12 and small molecules/factors namely Activin A (100 ng/mL), CHIRR9901 (2 μM), LDN193189 (200 nM), PI-103 (25 nM), Insulin-Transferrin-Selenium (ITS-G) (1:1000), KSR (0.05%), Pen strep (100×), and NEAA (100×).

On day 5, Experiment 23 Media B containing DMEM-F12 and small molecules/factors namely Activin A (100 ng/mL), LDN193189 (200 nM), PI-103 (25 nM), Insulin-Transferrin-Selenium (ITS-G) (1:1000), KSR (0.05%), Pen strep (100×), NEAA (100×) was added.

Differentiation of Definitive Endoderm (DE) into Anterior Foregut Endoderm (AFE)

On day 6, the aggregates were seeded on Geltrex-coated 24-well plates. On day 6-8, Experiment 23 AFE Media* containing DMEM-F12 and small molecules/factors namely SB431542 (10 μM), LDN193189 (200 nM), Pen Strep (100×), Insulin-Transferrin-Selenium (ITS-G) (1:1000), KSR (0.05%), NEAA (100×), B27 without VIT A (0.5×), BME (100×) was added.

### Differentiation of Anterior Foregut Endoderm (AFE) to Ventral Pharyngeal Endoderm (VPE)

On day 9, aggregates were seeded on Geltrex-coated 24-well plates. On day 9-10, Experiment 23 VPE1 Media containing DMEM-F12 and small molecules/factors namely SB431542 (10 uM/ml), Retinoic Acid (0.1 μM), FGF8b (50 ng/ml), Insulin-Transferrin-Selenium (ITS-G) (1:1000), KSR (0.0025%), Pen strep (100×), NEAA (100×), B27-without VIT A (0.5×), and BME (100×) was added. On day 10, only half the media was added. On day 11-12, Experiment 23 VPE2 Media containing DMEM-F12 and small molecules/factors namely Retinoic Acid (0.1 μM), FGF8b (50 ng/ml), Noggin (50 ng/ml), CHIR99021 (2 μM), Ascorbic Acid (30 μM), Insulin-Transferrin-Selenium (ITS-G) (1:1000), KSR (0.0025%), Pen strep (100×), NEAA (100×), B27-without VIT A (0.5×), Glutamax (100×), BME (100×) and N2(100×) was added.

### Differentiation of Ventral Pharyngeal Endoderm (VPE) to Thymic Epithelial Progenitors (TEP)

On day 13-16, Experiment 23 TEP Media containing DMEM-F12 and small molecules/factors namely FGF8b (50 ng/ml), FGF10 (50 ng/ml), BMP4 (50 ng/ml), CHIRR99 (2 μM), Ascorbic Acid (30 μM), Insulin-Transferrin-Selenium (ITS-G) (1:1000), KSR (0.0025%), Pen strep (100×), NEAA (100×), B27-without VIT A (0.5×), Glutamax (100×), BME (100×) and N2(100×) was added. On day14, 15 and 16 only half the media was changed. On day17-20, TEC Media containing, DMEM-F12 and small molecules/factors namely BMP4 (50 ng/ml), FGF8b (50 ng/ml), Ascorbic Acid (30 μM), Retinoic Acid (0.1 μM), Insulin-Transferrin-Selenium (ITS-G) (1:1000), KSR (0.0025%), Pen strep (100×), NEAA (100×), B27-without VIT A (0.5×), N2 (100×), Glutamax (100×), and BME (100×). All the Media was changed daily.

Addition of CHIRR99 to Experiment 23 Medium B was found to enhance differentiation.

### Example 12. Optimizing DE Differentiation

Experiment 21 and Experiment 23 described in Example 11 were designed to identify optimal conditions for 3D differentiation to DE. Inclusion of Wnt activator in differentiation medium in Experiment 23 increased differentiation efficiency to DE compared to without Wnt activator in Experiment 21 and Experiment 22. Sox17/FoxA2 double positive were higher in Experiment 23, whereas there were more Sox17/FoxA2 negative cells in Experiment 21 and Experiment 22.

Higher expression of EPCAM/FoxA2 double positive cells was higher in Experiment 23. Similarly SOX2/Tra1-60 expression was higher in levels of Tra1-60/SOX2 double negative cells, whereas a persistent small population of Tra1-60/Sox2 double positive cells was observed in Experiment 21/22 (likely undifferentiated iPSC cells).

CXCR4 expression was lower in Experiment 23, compared to Experiment 21 and Experiment 22.

In summary, the differentiation of pluripotent state of iPSCs was found to be more complete in Experiment 23.

### Example 13. 2D Culture of TEPs

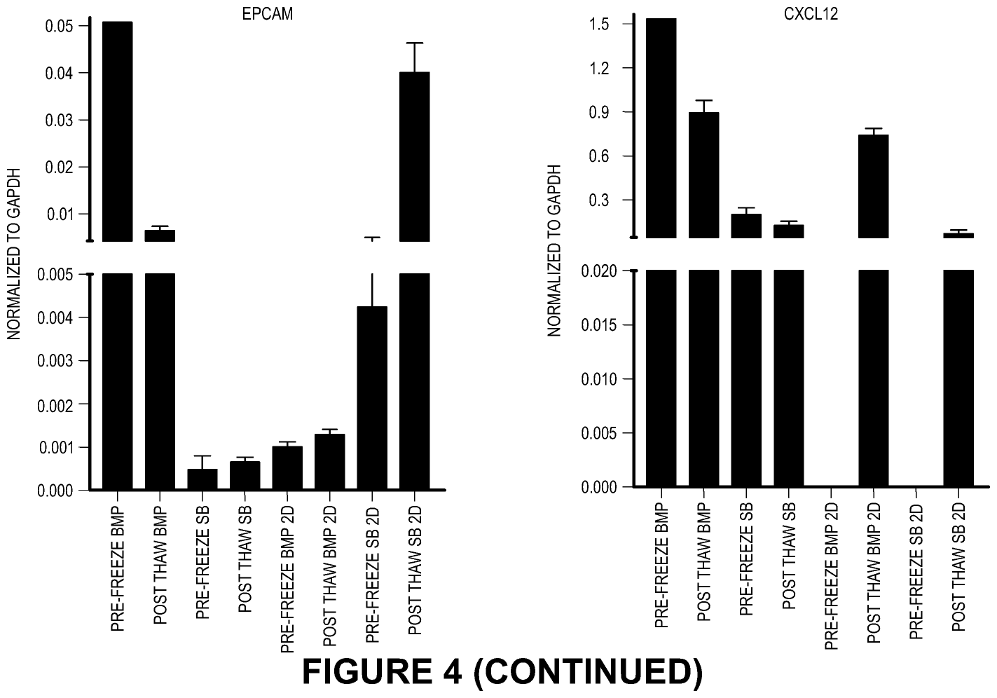

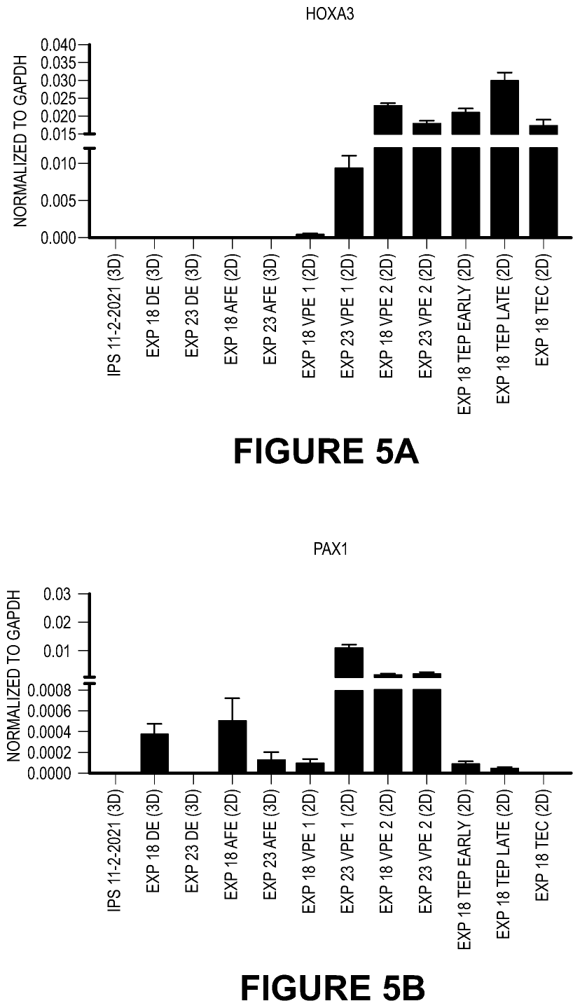

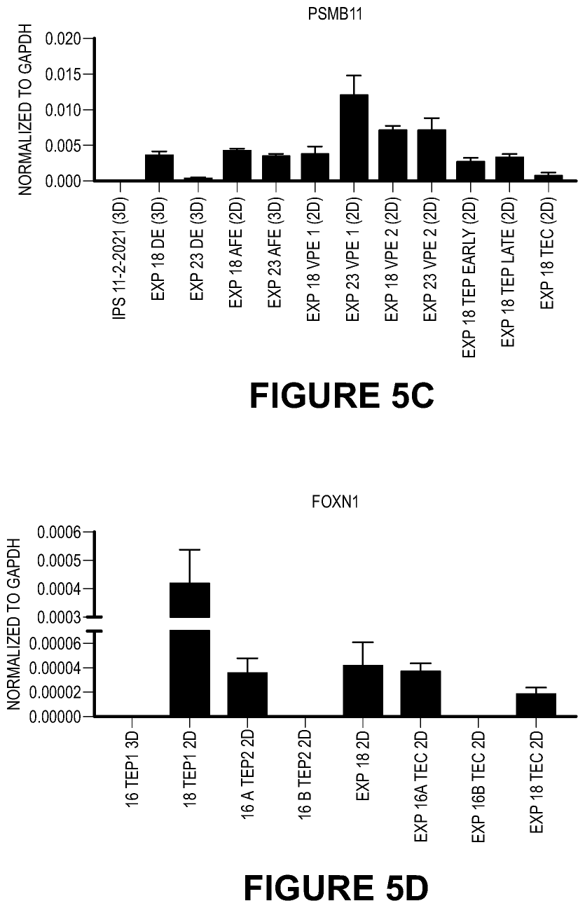

Spheroids (also referred to herein as “3D culture”) were induced to differentiate from iPSC to DE and AFE in 3D condition. A subsequent 2D culture step (i.e. culturing cells in Geltrex) was found to be important to induce to final FoxN1 positive TEP cells. Expression of markers associated with iPSC to TEP differentiation were measure at progressive stages of differentiation for the various differentiation protocols described in Example 11. Several conditions were found to be important, using key developmental genes as markers. Timing of transition to 2D by inducing AFE in 3D versus AFE in 2D showed that inducing AFE in 3D was better than inducing AFE in 2D for further differentiation of iPSC to TEPs as marked by upregulation of key VPE markers HOXA3 (FIG. 5A), Pax1 (FIG. 5B) and PSMB11 (FIG. 5C) in the VPE1 stage. AFE in 3D Experiment 23 condition showed a significantly higher expression of HOXA3, PAX1, and PSMB11 expression by VPE1 stage compared to AFE in 2D culture in Experiment 18.

Induction to FoxN1+TEP was observed under 2D conditions i.e., culture in Geltrex containing medium (FIG. 5D). Transition from spheroids to 2D culture either by allowing spheroids to adhere to Geltrex or by dissociating spheroids into single cell and replating on Geltrex was conducted. This analysis showed that allowing the spheroids to adhere to Geltrex resulted in more robust and consistent differentiation to FOXN1+TEPs (FIG. 5D). High expression of FOXN1+TEP/TEC was only observed under 2D culture conditions. In Experiment 16, 3D culture of TEPs did not result in FOXN1 expression compared to 2D culture of TEPs in Experiment 18. Transitioning TEPs from 3D to 2D culture in Experiment 16 showed induction of FOXN1 expression. In Experiment 16, 3D spheroids were transferred to 2D culture condition, either by allowing spheroids to adhere to Geltrex (Experiment 16A) or by dissociating spheroids into single cell before replating onto Geltrex (Experiment 16B). As shown in FIG. 5D, conditions of Experiment 16A resulted in a robust and consistent differentiation of FOXN1 positive TEPs.

### Example 14. Ectopic Expression of FOXN1

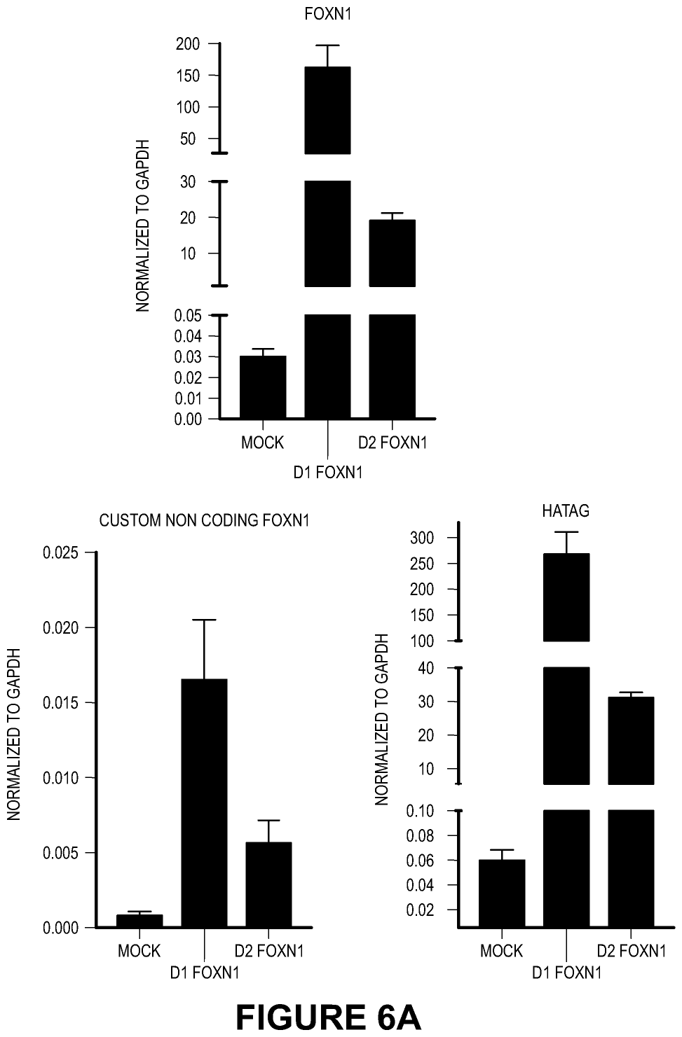

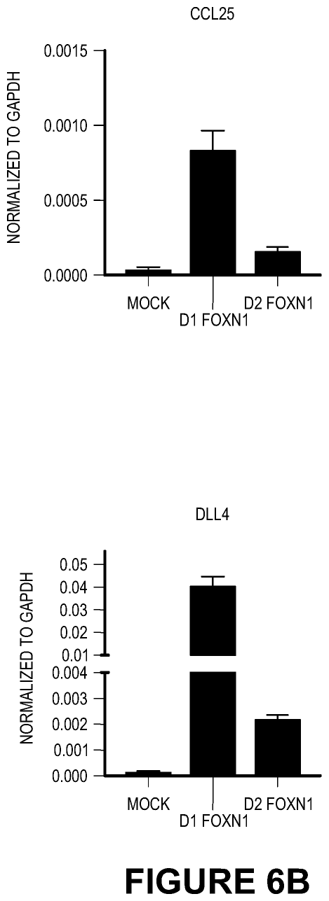

The effect of ectopic expression of FOXN1 on TEP differentiation was tested. TEPs were transfected with FOXN1. Expression of FOXN1 was measured using primers that target the coding region of FOXN1. As shown in FIG. 6A, transfection of FOXN1 mRNA generated a high level of FOXN1 mRNA that include exogenous and endogenous mRNA molecules. Using primers that target only mRNA with 3′UTR, robust expression of endogenous FOXN1 mRNA induced by exogenously expressed mRNA was observed. Using primers that target the HA-tag appended to the exogenous mRNA, expression of exogenous mRNA in TEPs was confirmed (FIG. 6A).

The high expression of exogenous and endogenous FOXN1 mRNA led to robust induction of direct targets of FoxN1 e.g. CCL25, DLL4 (FIG. 6B).

### Example 15. Differentiation of iPSC to Thymic Cells

### Experiment 27

Expansion of iPSC Line in Suspension Culture

Before initiating the differentiation protocol, iPSCs were cultured 3-4 days in suspension till they reached a diameter of ˜300-400 microns. iPSCs were then passaged using Accutase to turn aggregates into single cells. 2.5 million single iPSC cells were placed in suspension in 6 well ultra-low adhesion plates. iPSCs were cultured in stem scale media containing 10 μM of ROCK inhibitor, Y27632 at 37° C.

1 ml supernatant was taken from the plates and 1 ml of pre-warmed stem scale media was added to the culture. On day 2, the supernatant along with spheroids were transferred into a 15 ml conical tube and centrifuged at 250 G for 5 minutes. The spheroids were washed with PBS and resuspended in 1 ml of Media A to each well of a 6-well low adhesion plate.

Differentiation of iPSCs into Definitive Endoderm (DE)

Differentiation of iPSCs into DE requires proper timing for introducing small molecules and growth factors. On days 1-2, iPSCs were cultured in Media A (Basal Media: DMEM-F12, Activin A (100 ng/mL), CHIR99021 2 μM, Insulin-Transferrin-Selenium (ITS-G) (1:1000), KSR (0.05%), Pen strep (1:100), NEAA (1:400)) with PI-103 (25 nM) added. On day 2, 1 ml of media was removed and 1 ml of freshly prepared Media A+PI-103 was added. On days 3-5, Media B (DMEM-F12, Activin A (100 ng/mL), LDN193189 (200 nM), Insulin-Transferrin-Selenium (ITS-G) (1:1000), KSR (0.05%), Pen strep (1:200), NEAA (1:400)) and PI-103 (25 nM) were added. On day3, CHIRR9901 (2 μm) was added to Media B. On days 3-5, 1 ml of media was removed and 1 ml of freshly prepared media was added.

Differentiation of Definitive Endoderm (DE) into Anterior Foregut Endoderm (AFE)

On day 6 the supernatant together with the spheroids were transferred into 15-ml conical tubes. The spheroids were centrifuged at 250 G for 5 minutes at room temperature and the supernatant was removed. The spheroids were washed with PBS and plated into 6-well low adhesion plates containing AFE Media-2 comprising DMEM-F12 and factors/small molecules namely SB431542 (10 μM), LDN193189 (200 nM), Ascorbic Acid (10 μM), Penicillin-streptomycin (1:200), N2 (1:100), Glutamax (1:100), BME (1:100), Insulin-Transferrin-Selenium (ITS-G) (1:1000), KSR (0.05%), NEAA (100×), B27 (without RA) (1:200), in addition to FGF8b (50 ng/ml).

### Differentiation of Anterior Foregut Endoderm (AFE) to Ventral Pharyngeal Endoderm (VPE)

On day 8, the supernatant alongside spheroids were transferred into 15-ml conical tubes. The spheroids were centrifuged at 250G for 5 minutes and the supernatant was aspirated. The spheroids were washed with PBS and resuspended in 50 μl of VPE1a media comprising DMEM-F12, and with factors/small molecules SB431542 (10 uM/ml), Retinoic Acid (0.1 μM), FGF8b (50 ng/ml), Retinoic acid (0.1 μM), Insulin-Transferrin-Selenium (ITS-G) (1:1000), KSR (0.0025%), Penicillin streptomycin (1:200), NEAA (100×) and B27-without VIT A (0.5×), Ascorbic Acid (10 μM), Glutamax (1:100), BME (1:100), N2(1:100) and NEAA (1:400). The spheroids were then added to 24-well plates coated with Geltrex (1:100).

On day 9, only half the media was changed. On day 10-11, VPE2b media containing DMEM-F12, Retinoic Acid (0.1 μM), FGF8b (50 ng/ml), Noggin (50 ng/ml), CHIR99021 (2 μM), Ascorbic Acid (10 μM), Insulin-Transferrin-Selenium (ITS-G) (1:1000), KSR (0.0025%), Penicillin streptomycin (100×), and NEAA (100×) B27-without VIT A (0.5×) was added, Glutamax (1:100), BME (1:100) and N2 (1:100). On Day 10 and 11, equal amount of freshly made VPE2b media (500 ul) was added gently without disturbing the aggregates

### Differentiation of Ventral Pharyngeal Endoderm (VPE) to Thymic Epithelial Progenitors and (TEP) and Thymic Epithelial Cells (TEC)

For this differentiation step, VPE cells were cultured in thymic cell media e.g. TEP media or TEC media. On day 12-19, Experiment 27 TEP Media containing DMEM-F12, and with factors/small molecules FGF8b (50 ng/ml), BMP4 (50 ng/ml), CHIRR99 (2 μM), Ascorbic Acid (10 μM), Insulin-Transferrin-Selenium (ITS-G) (1:1000), KSR (0.0025%), Penicillin streptomycin (1:200), NEAA (100×), in addition to FGF10 (50 ng/ml), B27 (without VIT A) (1:200), Glutamax (1:100), BME (1:100), N2(1:100) was added to the cells.

On day 12, equal amount of freshly made TEP media was added gently without disturbing the aggregates. On days 13-19, 50% of the supernatant was removed and freshly prepared Experiment 27 TEP Media was added.

For days 20-23, cells were resuspended in Experiment 27 TEC media containing DMEM-F12, BMP4 (50 ng/ml), FGF8b (50 ng/ml), Ascorbic Acid (10 μM), Insulin-Transferrin-Selenium (ITS-G) (1:1000), KSR (0.0025%), Pen strep (100×), B27-without VIT A (0.5×), N2 (100×), Glutamax (100×), BME (100×) and NEAA (100×) in addition to FGF10 (50 ng/ml), FGF7/KGF (50 ng/ml), RANKL/TRANCE (50 ng/ml), and CHIRR99 (2 μM).

### Experiment 29

Expansion of iPSC Line in Suspension Culture

Before initiating the differentiation protocol, iPSCs were cultured 3-4 days in suspension till they reached a diameter of ˜300-400 microns. iPSCs were then passaged using Accutase to turn aggregates into single cells. 2.5 million single iPSC cells were placed in suspension in 6 well ultra-low adhesion plates. iPSCs were cultured in stem scale media containing 10 μM of ROCK inhibitor, Y27632 at 37° C.

1 ml supernatant was taken from the plates and 1 ml of pre-warmed stem scale media was added to the culture. On day 2, the supernatant along with spheroids were transferred into a 15 ml conical tube and centrifuged at 250 G for 5 minutes. The spheroids were washed with PBS and resuspended in 1 ml of Media A to each well of a 6-well low adhesion plate.

Differentiation of iPSCs into Definitive Endoderm (DE)

Differentiation of iPSCs into DE requires proper timing for introducing small molecules and growth factors. On days 1-2, iPSCs were cultured in Media A (Basal Media: DMEM-F12, Activin A (100 ng/mL), CHIR99021 2 μM, Insulin-Transferrin-Selenium (ITS-G) (1:1000), KSR (0.05%), Pen strep (1:100), PI-103 (25 nM), NEAA (1:400). On day 2, 1 ml of media was removed and 1 ml of freshly prepared media was added. On days 3-5, cells were incubated with Media B (DMEM-F12, Activin A (100 ng/mL), LDN193189 (200 nM), PI-103 (25 nM), Insulin-Transferrin-Selenium (ITS-G) (1:1000), KSR (0.05%), Pen strep (1:200), and NEAA (1:400). On days 4-5, 1 ml of Media B was removed and 1 ml of freshly prepared Media B was added.

Differentiation of Definitive Endoderm (DE) into Anterior Foregut Endoderm (AFE)

On day 6 the supernatant together with the spheroids were transferred into 15-ml conical tubes. The spheroids were centrifuged at 250 G for 5 minutes at room temperature and the supernatant was removed. The spheroids were washed with PBS and plated into 6-well low adhesion plates containing AFE Media-2 (Experiment 29A) or Geltrex coated plates (Experiment 29B). AFE medium utilized for the experiment contained Basal Media: DMEM-F12, LDN193189 (200 nM), SB431542(10 μM), FGF8b (50 ng/ml), Ascorbic Acid (10 μM), Insulin-Transferrin-Selenium (ITS-G) (1:1000), Pen Strep (1:200), B27 (without RA) (1:200), N2 (1:100), Glutamax (1:100), BME (1:100) and NEAA (1:400). On day 7 and 8, 1 ml of AFE media was removed and 1 ml of freshly prepared media was added.

### Differentiation of Anterior Foregut Endoderm (AFE) to Ventral Pharyngeal Endoderm (VPE)

On day 9, for Experiment 29A, the supernatant alongside spheroids were transferred into 15-ml conical tubes. The spheroids were centrifuged at 250G for 5 minutes at room temperature and the supernatant was aspirated. The spheroids were washed with PBS, resuspended in VPE1 media and plated onto 24-well plates coated with Geltrex (1:100). Both Experiment 29A and 29B cells were cultured from days 9-10 in VPE1 media which included basal Media: DMEM-F12, SB431542 (10 uM), FGF8b (50 ng/ml), Retinoic Acid (0.1 μM), Ascorbic Acid (10 μM), Insulin-Transferrin-Selenium (ITS-G) (1:1000), KSR (0.05%), Pen strep (1:200), B27 (without VIT A) (1:200), Glutamax (1:100), BME (1:100), N2(1:100) and NEAA (1:400). On day 9, equal amount of freshly made VPE1 media (500 ul) was added gently without disturbing the aggregates.

On day 10, 50% of supernatant was removed and freshly prepared VPE1 media (500 ul) was added per well. On days 11-12, media was replaced with VPE2 media containing Basal Media: DMEM-F12, Noggin (50 ng/ml), CHIR99021 (2 μM), FGF8b (50 ng/ml), Retinoic Acid (0.1 μM), Ascorbic Acid (10 μM), Insulin-Transferrin-Selenium (ITS-G) (1:1000), KSR (0.0025%), Pen strep (1:200), NEAA (1:400), B27 (without VIT A) (1:200), Glutamax (1:100), BME (1:100), and N2(1:100). On days 11 and 12, equal amount of freshly made VPE2 media (500 ul) was added gently without disturbing the aggregates.

### Differentiation of Ventral Pharyngeal Endoderm (VPE) to Thymic Epithelial Progenitors (TEP)

For this differentiation step, VPE cells were cultured in thymic cell media e.g. TEP media or TEC media. On days 13-18, media was replaced for Experiment 29 TEP media containing Basal Media: DMEM-F12, FGF10 (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), and NEAA (1:400). On day 13, equal amount of freshly made TEP media (500 ul) was added gently without disturbing the aggregates. On day 14-18, 50% of supernatant was removed and freshly prepared TEP media (500 ul) was added per well. On days 19-22, media was replaced with TEC media containing Basal Media: DMEM-F12, FGF10 (50 ng/ml), FGF7/KGF (50 ng/ml), RANKL/TRANCE (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), NEAA (1:400), Glutamax (100×), and BME (100×). On days 20-22, 50% of supernatant was removed and freshly prepared TEC media (500 ul) was added per well. On day 22, Experiment 29B was reseeded as single cells in suspension (3D) and on Geltrex coated plates (2D). Remaining cells were frozen. Frozen Experiment 29B were thawed in suspension and maintained in TEC media.

### Experiment 30

Expansion of iPSC Line in Suspension Culture

Before initiating the differentiation protocol, iPSCs were cultured 3-4 days in suspension till they reached a diameter of ˜300-400 microns. iPSCs were then passaged using Accutase to turn aggregates into single cells. 2.5 million single iPSC cells were placed in suspension in 6 well ultra-low adhesion plates. iPSCs were cultured in stem scale media containing 10 μM of ROCK inhibitor, Y27632 at 37° C.

1 ml supernatant was taken from the plates and 1 ml of pre-warmed stem scale media was added to the culture. On day 2, the supernatant along with spheroids were transferred into a 15 ml conical tube and centrifuged at 250 G for 5 minutes. The spheroids were washed with PBS and resuspended in 1 ml of Media A to each well of a 6-well low adhesion plate

Differentiation of iPSCs into Definitive Endoderm (DE)

Differentiation of iPSCs into DE requires proper timing for introducing small molecules and growth factors. On days 1-2, iPSCs were cultured in Media A (Basal Media: DMEM-F12, Activin A (100 ng/mL), CHIR99021 2 μM, Insulin-Transferrin-Selenium (ITS-G) (1:1000), KSR (0.05%), Pen strep (1:100), PI-103 (25 nM), and NEAA (1:400)). On days 3-5, cells were treated with Media B containing Basal Media: DMEM-F12, Activin A (100 ng/mL), LDN193189 (200 nM), PI-103 (25 nM), Insulin-Transferrin-Selenium (ITS-G) (1:1000), KSR (0.05%), Pen strep (1:200) and NEAA (1:400).

Differentiation of Definitive Endoderm (DE) into Anterior Foregut Endoderm (AFE)

On day 6 the supernatant together with the spheroids were transferred into 15-ml conical tubes. The spheroids were centrifuged at 250 G for 5 minutes at room temperature and the supernatant was removed. The spheroids were washed with PBS and plated into 6-well low adhesion plates containing AFE Media-2 comprising DMEM-F12 and factors/small molecules namely SB431542 (10 μM), LDN193189 (200 nM), Ascorbic Acid (10 μM), Penicillin-streptomycin (1:200), N2 (1:100), Glutamax (1:100), BME (1:100), NEAA (100×), B27 (without RA) (1:200), in addition to FGF8b (50 ng/ml).

### Differentiation of Anterior Foregut Endoderm (AFE) to Ventral Pharyngeal Endoderm (VPE)

On day 9, the supernatant alongside spheroids were transferred into 15-ml conical tubes. The spheroids were centrifuged at 250G for 5 minutes at room temperature and the supernatant was aspirated. The spheroids were washed with PBS, resuspended in VPE1 media and plated onto 24-well plates coated with Geltrex (1:100). Cells were cultured from days 9-11 in VPE1 media which included Basal Media: DMEM-F12, SB431542 (10 uM), FGF8b (50 ng/ml), Retinoic Acid (0.1 μM), Ascorbic Acid (10 μM), Insulin-Transferrin-Selenium (ITS-G) (1:1000), KSR (0.05%), Pen strep (1:200), B27 (without VIT A) (1:200), Glutamax (1:100), BME (1:100), N2(1:100), NEAA (1:400). On day 10, equal amount of freshly made VPE1 media (500 l) was added gently without disturbing the aggregates. On day 11, 50% of supernatant was removed and freshly prepared VPE1 media (500 ul) was added per well. On days 12-13, the media was removed and replaced with VPE2 media containing Basal Media: DMEM-F12, Noggin (50 ng/ml), CHIR99021 (2 μM), FGF8b (50 ng/ml), Retinoic Acid (0.1 μM), Ascorbic Acid (10 μM), Insulin-Transferrin-Selenium (ITS-G) (1:1000), KSR (0.0025%), Pen strep (1:200), NEAA (1:400), B27 (without VIT A) (1:200), Glutamax (1:100), BME (1:100), and 2(1:100).

On Day 13, equal amount of freshly made VPE2 media (500l) was added gently without disturbing the aggregates.

### Differentiation of Ventral Pharyngeal Endoderm (VPE) to Thymic Epithelial Progenitors (TEP)

For this differentiation step, VPE cells were cultured in thymic cell media e.g. TEP media or TEC media. On days 14-16, cells were treated with Experiment 30 TEP media including DMEM-F12, FGF10 (50 ng/ml), FGF7/KGF (50 ng/ml), RANKL/TRANCE (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), and NEAA (1:400). On days 15 and 16 new TEP media was added to the cells. From days 17-22, cells were treated with TEC media containing DMEM-F12, FGF10 (50 ng/ml), FGF7/KGF (50 ng/ml), RANKL/TRANCE (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR, (0.0025%), Pen strep (1:200), B27 (without VIT A) (1:200), Glutamax (1:100), BME (1:100), N2(1:100), NEAA (1:400), Glutamax (100×), and BME (100×).

On days 17-22, 50% of supernatant was removed and freshly prepared TEC media (500l) was added per well. On day 22, the cells were treated with Accutase to make prepare single cells and transplanted into mice.

### Example 16. FOXN1 Expression in iPSC Derived Thymic Cells

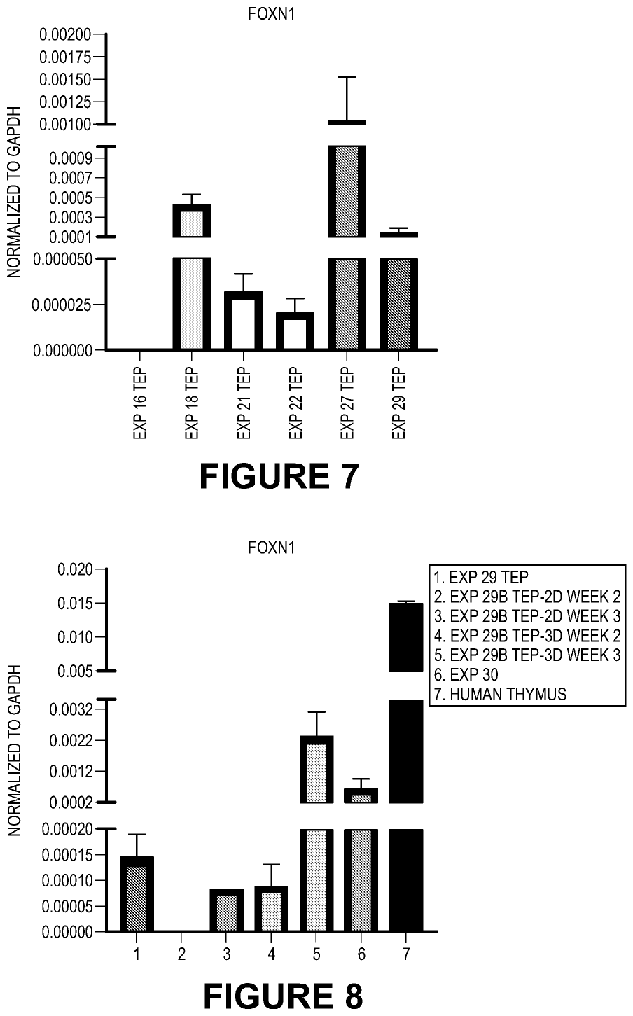

The culture of cells in 2D versus 3D aggregates in suspension is yet another variable that needs to be tuned to obtain optimal thymic cell populations from iPSCs. Other variables include the growth factors and small molecules utilized in culture at different stages. Transition from 3D aggregates to culturing in 2D at the Ventral Pharyngeal Endoderm (VPE) stage can promote the expression of markers such as HOXA3, PAX9, PAX1. iPSC derived thymic cells were consistently found to express thymic cell marker, FOXN1, in different derivations (FIG. 7). TEP expressing FOXN1 at levels, normalized to GAPDH, from 0.000025 to 0.0001 were observed in all experiments tested. Thus the protocol of 16-18 days allow generation of FOXN1 positive cells compared to other protocols that can be as long as 30 days.

### Example 17. Optimizing Growth Factors and Culture Conditions

In experiments 16 through 23, the expression of FOXN1 and other markers expression by Day5 of TEP differentiation. In these experiments, longer culture of TEP for another week lead to a downregulation of FOXN1. Growth Factors such as FGF7(KGF), FGF10, and RANKL have been described to be important for the late maturation of TEPs to TECs. The addition of FGF7, FGF10, KGF and RANKL to TEP medium for supporting TEP cultures beyond 5 days was tested. By supplementing the TEP medium with FGF7, FGF10 and RANKL in Experiment 29 and Experiment 30, the culture of TEPs was extended beyond 5 days and up to 2 weeks while still maintaining the expression of FOXN1. To facilitate scale up manufacturing of iPSC derived thymic cell product, an important objective is to be able to establish a differentiation protocol that is fully in 3D suspension. The feasibility of maintaining TEP in 3D suspension culture was also explored in these experiments. At TEP stage, single cells were harvested from culture plates and resuspended in TEP medium as aggregates into 3D. FOXN1 expression levels in TEP from different experiments are shown in FIG. 8. In Exp 29-TEPs, FOXN1 expression was detected at week 1 of TEP stage. When TEPs were reseeded into 2D culture, no FOXN1 expression was detected after 1 week but FOXN1 was detected by week 2 in culture. In Exp29B-TEP2-3D, where single cell TEP were resuspended into 3D-aggregate culture, FOXN1 expression was detected already by 1 week and became even higher by second week. In Experiment 30, comparable FOXN1 expression was detected even prior to extended culture in 2D or 3D.

### Example 18. Effect of Freezing and Thawing TEPs

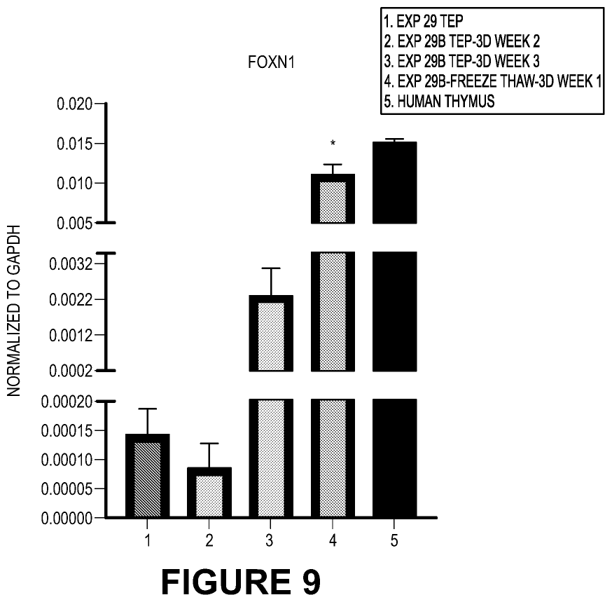

The ability to freeze, thaw, and recover iPSC-TEPs would provide significant logistic value for supporting the use and delivery of iPSC-TEPs to clinical sites. The feasibility of freezing and thawing iPSC-TEPs. Single cell suspension of Experiment 29B TEP1 cells were frozen then thawed into 3D suspension in TEP medium. Aggregates were observed on day 1 that increased in size and compactness. By day 8 aggregates reached 200-300 μm in diameter. FOXN1 expression of Exp29B TEPs under different culture conditions are shown in FIG. 9. Frozen-and-thawed “Exp29B-FT-TEP” cells in 3D aggregates expressed FOXN1 at day 8 after thaw at levels comparable to the human thymus. The Exp29B-FT-TEP compared to 29B-TEP in 3 D for 3 weeks.

### Example 19. In Vivo Transplantation of Thymic Cell Populations

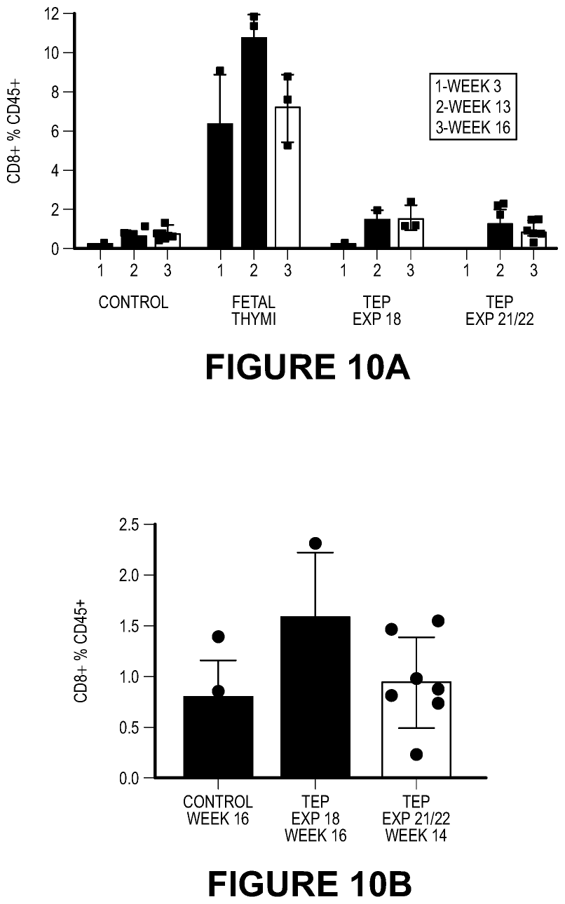

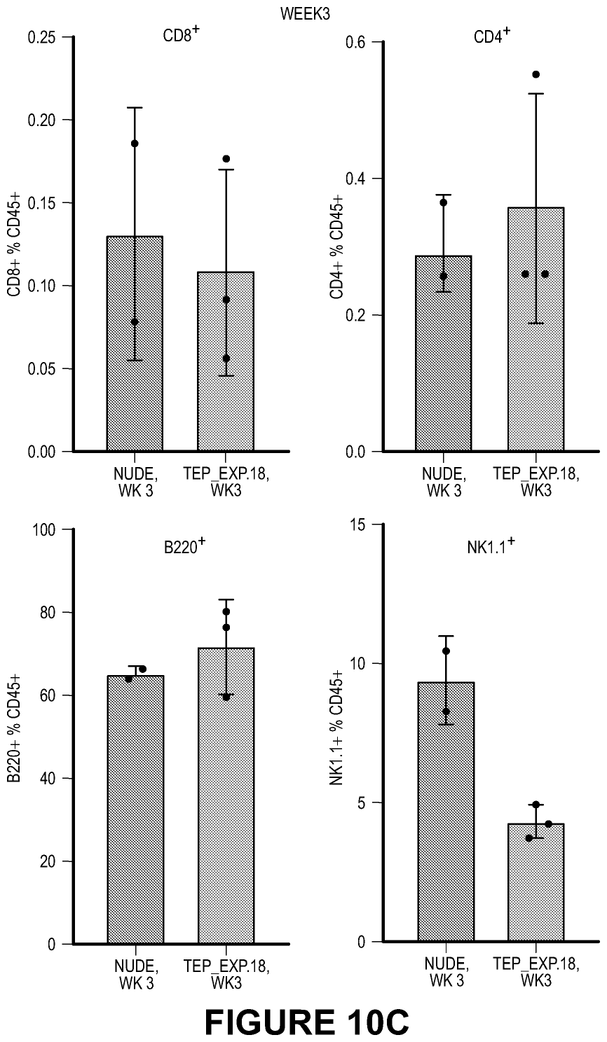

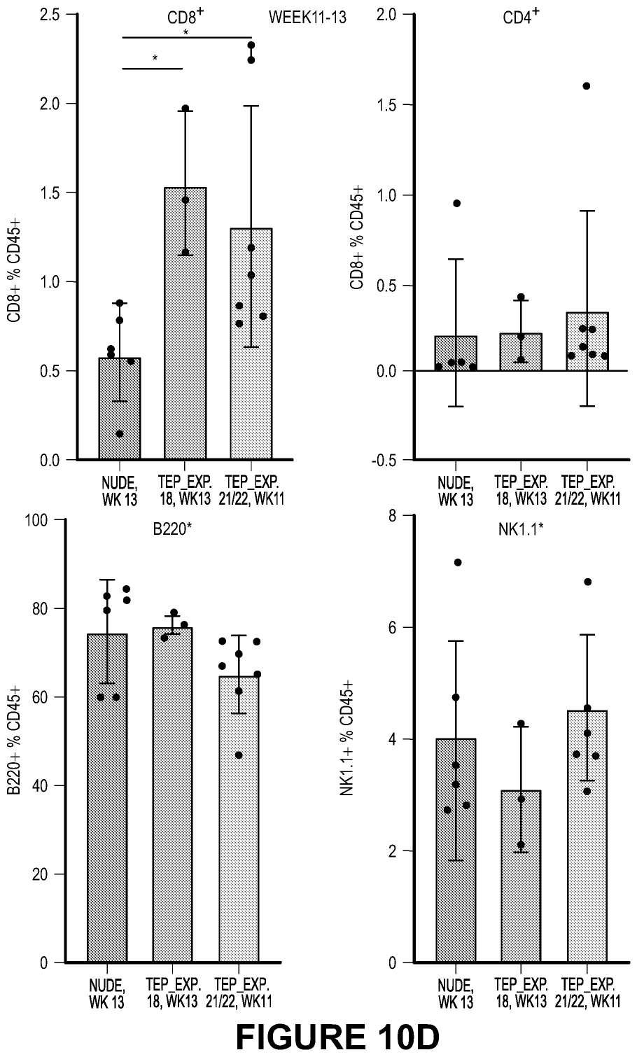

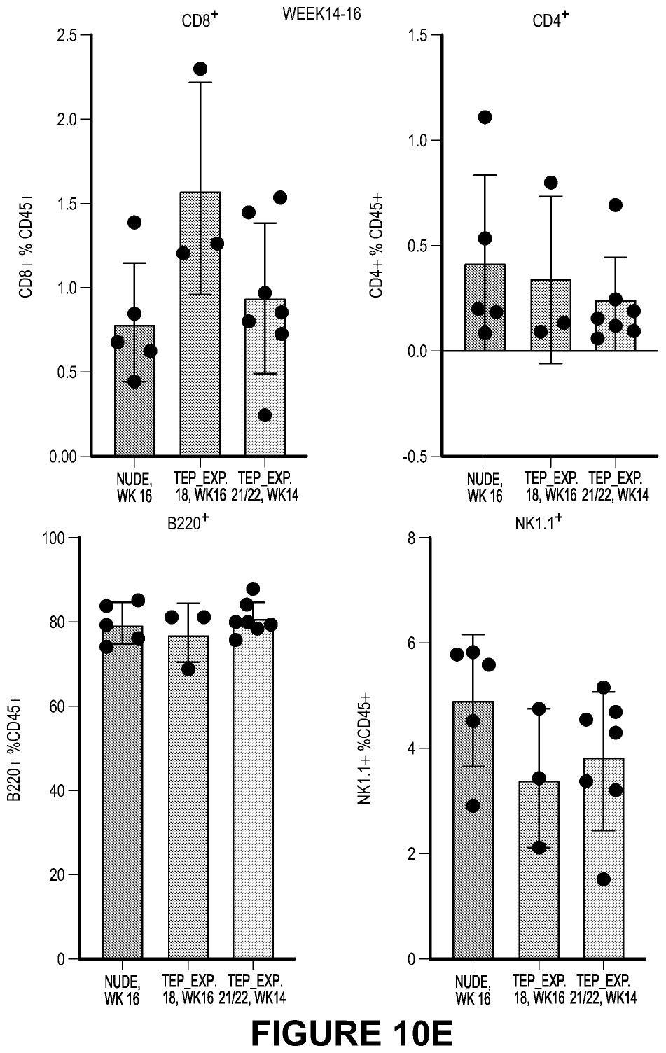

1-4×10⁶ iPSC derived TEPs were suspended in 25 μL of Geltrex and injected into sub renal capsule of 6-week-old nude mice. 3 animals were transplanted with iPSC derived thymic cells from Experiment 18 and 8 animals were transplanted with cells from Experiment 21/22. Blood samples were obtained from transplanted mice at week 3, week 11 and week 13 and hematopoietic cell numbers in post iPSC-TEP transplant mice was compared to mice with fetal thymus transplant and control non-transplanted animals. As shown in FIG. 10A, the percentage of CD8+% of CD45 cells were mice transplanted with TEPs increased from week 3 to week 13 and week 16. Overall, the percentage of CD8+ cells was higher than control at 16 weeks for Experiment 18 and 14 weeks for Experiment 21/22. It is noteworthy that some of the mice represented in FIG. 10B showing the CD8+ cells for Experiment 21/22 included two mice that did not incorporate the implanted cells for technical reasons. In contrast to CD8+% of CD45+ cells, other populations of hematopoietic cells including CD4+ cells, B220+, and NK1.1+ cells did not show any increase in percentage numbers compared to control and from week 3 to week 11-13 and week 14-16 for experiment 18 and experiment 21/22 (FIG. 10C, FIG. 10D and FIG. 10E). Together these data indicate that iPSC derived thymic cells described herein can promote lymphopoiesis.

### Example 20: Characterization of iPSC Derived TEPs Expressing FOXN

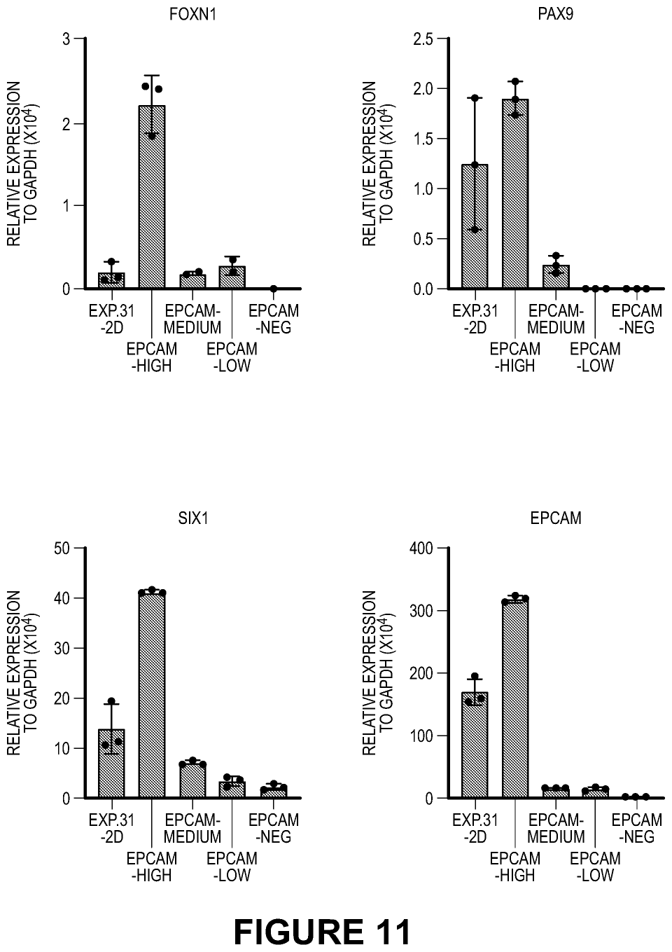

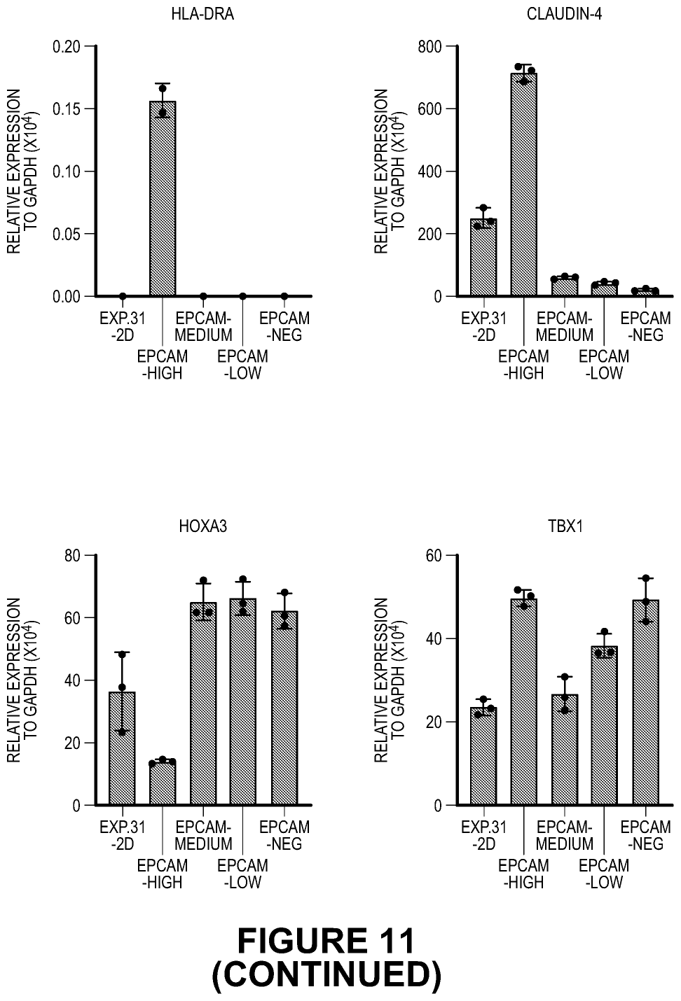

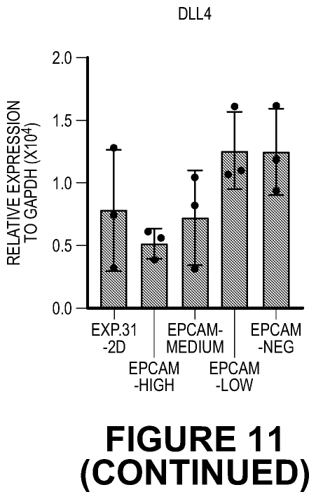

TEPs generated by Experiment 31 were sorted using anti-EPCAM antibody into four different fractions: EPCAM-high, -medium, -low, and -negative. The sorted fractions were analyzed by qPCR for various markers as shown (FIG. 11). FOXN1 was expressed almost exclusively in the EPCAM-high population. The genes involved in TEP development and maturation were also expressed in the EPCAM-high population, including PAX9, SIX1, and CLAUDIN4. HOXA3, TBX1 and DLL4 were expressed preferentially in the EPCAM-low to negative fraction.

### Example 21: Frequency of Markers in Thymic Epithelial Cells

Single-cell RNA-seq was performed for iPSC derived TEP prepared using differentiation protocols described herein. Data were analyzed from Experiments 7 (A-C), 21, 22, 23, 27, 29, and 30. Experiment 21 and Experiment 23 we also tested sample replicates to assess technical reproducibility. For Experiment 29 and Experiment 30, in addition to the 3D-to-2D protocol variations, the effects of freezing and thawing (FT) TEPs and replating into 2D (Exp30FT-2D) versus into 3D (Exp30FT-3D) was tested. In Experiment 29, varied condition Exp29A versus 29B were tested including, seeding into 2D (Exp29B-2D) versus freezing and thawing before replating in 3D (29BFT-3D). Of these experiments, the samples that were transplanted were FOXN1^{high} (Exp29BFT-3D), FOXN1^{low} (Exp30, Exp30FT-3D), and FOXN1-(Exp30FT-2D).

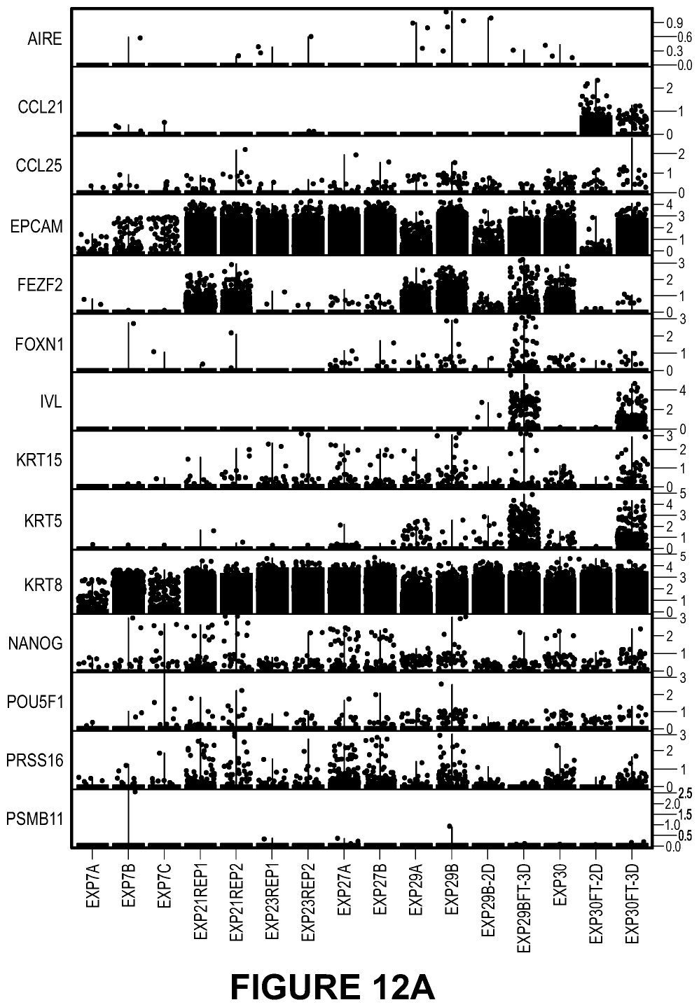

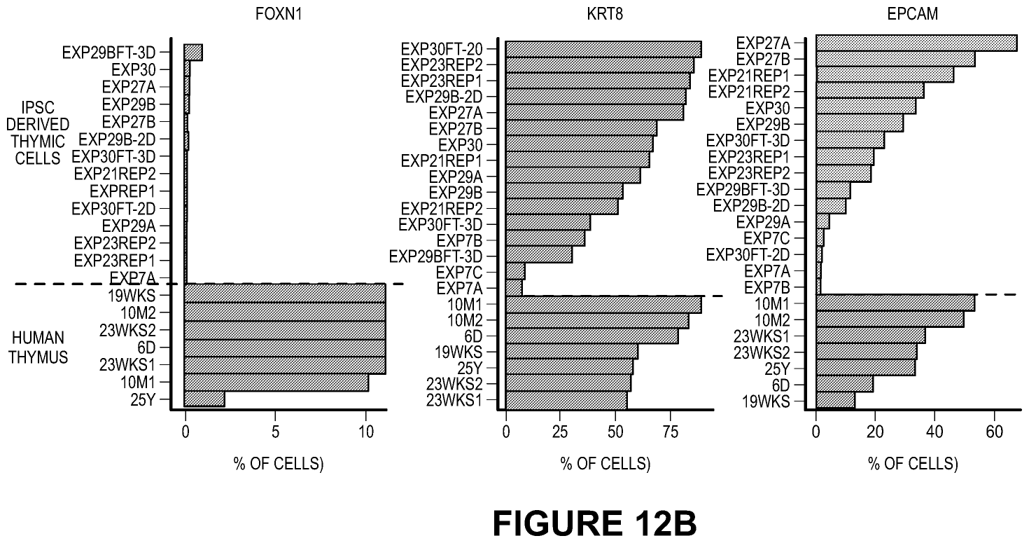

Single-cell transcriptomes of 122,436 cells from 16 iPSC derived TEC samples (14 different experiments) were integrated by Harmony and UMAP analysis was performed to ensure batch-effects were eliminated. Next, the gene expression distributions of selected genes for TECs (EPCAM, KRT8, FOXN1, IVL, and others), as well as pluripotency (POU5F1, NANOG) were plotted. The results are shown in FIG. 12A. EPCAM and Krt-8 were broadly expressed in high percentage across most experiments, while others are expressed in different frequencies across the experiments. From the violin plot, it was observed that FOXN1 was expressed more highly in TECs in Exp29BFT-3D and corresponded with the highest qPCR expression data. Exp29BFT-3D cells also showed the highest expression of markers KRT5 and Involucrin (IVL), genes that are specific for mTEC cells and cornified-like mTEC cells. Exp30FT-2D and Exp30FT-3D were the only experiments where cells expressing CCL21 a marker specific for mTEC cells were detected. Finally, it is worth noting that another mTEC marker FEZF2 is also highly expressed in some iPSC-TEP, including those with thymopoietic activity.

FIG. 12B shows the quantification of the percentage of FOXN1+, KRT8+, and EPCAM+ cells in iPSC differentiated thymic cells and thymic tissue samples. Consistent with the violin plot analysis, sample Exp29BFT-3D had the highest frequency of FOXN1+ cells. It is noteworthy that in human thymus, the highest level of FOXN1+ cells were in prenatal and newborns (10-15%) and by age 25 the frequency significantly reduces to 2-3%.

### Example 22: Detection Limits of Undifferentiated Pluripotent Stem Cells

To obtain an unbiased assessment of the status of the pluripotency gene regulatory network in the Exp29B3D sample, single-cell transcriptomes of the 7166 cells analyzed from this sample were classified using the CellNet training dataset, which contains high-quality bulk RNA-seq-derived pluripotency gene regulatory network (referred to as embryonic stem cell, esc) as well as high quality bulk RNA-seq data of 13 other cell types denoted in the panel. The analysis showed that other than cTEC and mTEC cells, the other major cell type in iPSC-TEP were neuroendocrine cells. These data also indicate the degree of sensitivity of this approach to detect cells with a pluripotent stem cell signature is very high. Additionally, the activation status of the embryonic stem cell network appeared to be very low in iPSC cells, suggestive of an overall downregulation of pluripotency programs.

The percentage of OCT4 (POU5F1) and Nanog positive cells was quantified based on all cells with log-normalized expression levels higher than zero. Overall, expression of POU5F1 and NANOG was less than 2% of cells from iPSC-TEP samples.

### Example 23: Correlation of In Vivo Thymopoietic Activity with FOXN1 Levels of Transplanted iPSC Derived TEPs

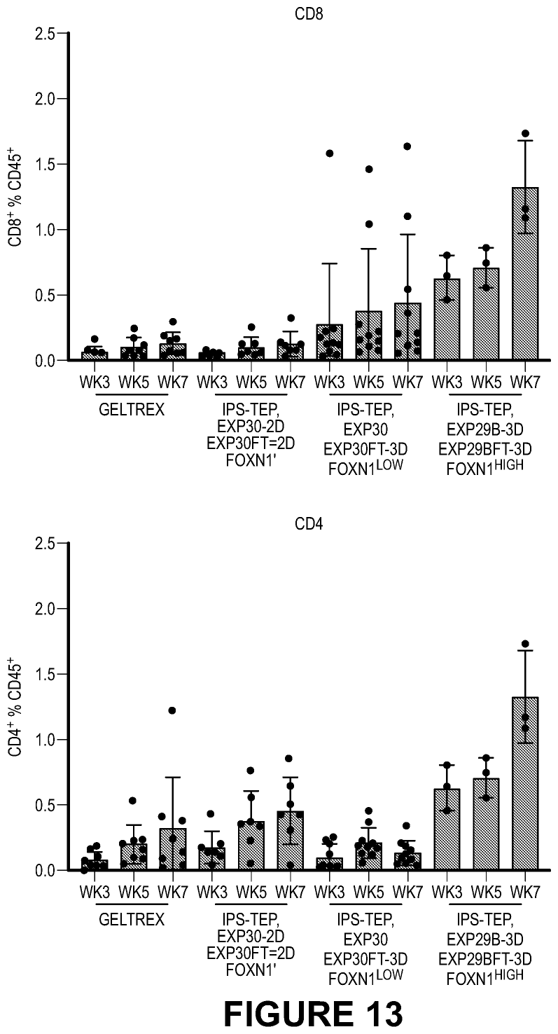

Three groups of iPSC derived TEP cells were transplanted with Geltrex into the renal subcapsular space: Cells with high FOXN1(18 to 20-fold relative to GAPDH, ×10⁴), low FOXN1 (5 to 8-fold relative to GAPDH, ×10⁴) and negative for FOXN1. Control animals were sham transplanted with Geltrex only. Evidence of restoration of thymopoiesis was measured by tracking emergence of single-positive CD4⁺ and CD8⁺ cells in peripheral circulation. FIG. 13 is a histogram showing the frequency of CD8⁺ or CD4⁺ cells in peripheral blood of animals at different time point after transplantation. CD4 and CD8 cells were observed at levels significantly above control animals in animals transplanted with FOXN1^{high} and, to lesser extent, FOXN1^{low} iPSC-TEPs. FOXN1 negative iPSC derived TEP showed no evidence of thymopoiesis. The data thus show a positive correlation between high levels of FOXN1 expression in iPSC derived TEP with capacity for restoring thymopoiesis.

### Example 24: Identity and Frequency of Subpopulations in iPSC Derived TEC Compared to Primary Human Thymus

Single-cell transcriptomes of 122,436 cells from 16 iPSC derived TEC samples (14 different experiments) were integrated by Harmony and UMAP analysis was performed to ensure batch-effects were eliminated. iPSC single-cell transcriptomics from all the iPSC derived TEP samples were then reference mapped to the reference single-cell atlas of human thymic cells generated by Bautista et al. (Bautista, J. L., et al. Nat Commun 12, 1096 (2021)). The mean classification score computed by Symphony was calculated for each cell type. An overall conservation of Symphony-predicted cell types across samples from different experiments was observed. For example, Exp29BFT-3D contained cells with strong classification as corneocyte-like mTECs, along with iPSC-derived thymic cells from sample Experiment 30 and Exp30FT-3D.

Next, the percentage of Symphony-predicted cell types across samples was calculated. In general, the most frequent cells found across all the samples were cTEC-high, cTEC-low, and neuroendocrine cells. The most frequent cells in the human thymus are also cTEC-high and cTEC-low cells.

### Example 25: Lymphopoiesis in iPSC Derived-TEP Transplanted Mice

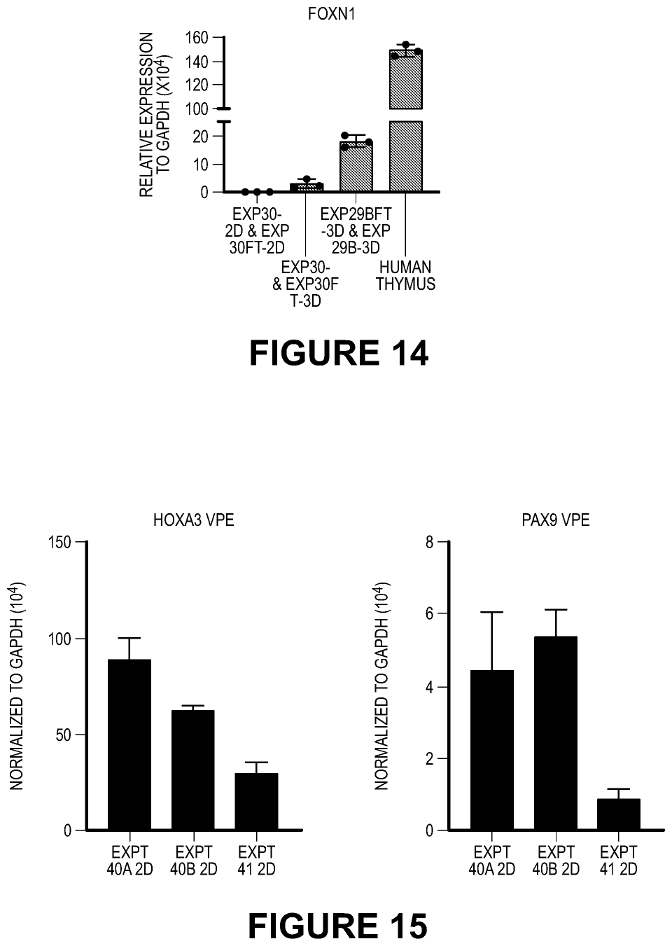

Fetal thymic fragments or iPSC derived-TEP were transplanted into the subrenal capsule space of 6-week-old athymic nude mice. 1-4×10⁶ iPSC derived TEPs were suspended in 15 μl of Geltrex and introduced into the subrenal capsule of 6-week-old nude mice. For fetal thymus, E13.5 fetal lobes were cultured for 5 days in 2DG to deplete T precursor cells. In one group of animals, 3 fetal lobes were transplanted as fragments into each kidney capsule. In another group, single-cell suspensions were prepared from 2DG treated fetal thymus and transplanted as a cell suspension. At different time points post-transplant, animals were bled for flow cytometry analysis of hematopoietic cells including T, B, and NK cells. Here we focus only on data for CD4+ and CD8+ T cells. To establish a benchmark for iPSC-TEP that can reconstitute thymopoiesis, iPSC-TEP expressing different levels of FOXN1 (FIG. 14) were tested. Transplanted murine fetal thymus cell suspension restored T lymphopoiesis detectable at week 6 but at slightly lower levels of CD8+ and CD4+ cells compared to fetal thymic fragments. iPSC-TEP with high FOXN1 showed CD8 and CD4 cells above the 1% level of control animals starting around week 6. The range of CD8 level (2-4%) is higher than that of CD4 (1-2%). FOXN1-low animals appeared to have some level of CD4 and CD8 cells above background. De novo thymopoiesis by iPSC-TEPs with low FOXN1 could not be determined due to outliers in the Geltrex control animals. The frequency of other hematopoietic cells such as B cells (B220) and NK cells (NK1.1) were similar between nude control and iPSC-TEP transplanted animals.

To assess the functional response of T cells generated from thymopoiesis of transplanted iPSC-TEPs, splenocytes were harvested from sacrificed animals and prepared for a cytokine release study. Cells were stimulated with PMA/Ionomycin, and cytokines produced in response to T cell activation signals were analyzed by flow staining. Under non-stimulated conditions, background staining levels were observed in splenocytes from transplanted mice. Activated splenocytes from Geltrex-transplanted showed IFNγ+ TNFα+ producing cells at low levels with 3.63%. In contrast CD4+ cells from iPSC derived TEC and fetal thymus transplant showed 12.3% and 5.8% of IFNγ+ TNFα+ producing cells, respectively. IFNγ or TNF expressing cells in CD8 population were also higher in both iPSC-TEC and fetal thymus transplant compared to Geltrex transplant. In conclusion. PMA/Ionomycin stimulation resulted in a greater release of cytokines IFNγ and TNFα compared to control splenocytes.

### Example 26: Differentiation of iPSC Cells to Thymic Cells (Experiment 31)

Expansion of iPSC Line in Suspension Culture

On day 0, mature iPSCs cultured (3 to 4 days in suspension (3D); 300-400 microns in diameter) were treated with pre-warmed Accutase to turn aggregates into single cells. Three million single iPSCs were plated in suspension into 6-well ultra-low adhesion plates in Stem scale media along with ROCK inhibitor Y27632 (10 μM). On day 1, pre-warmed stem scale media was added to the culture. On day 2, the supernatant and the spheroids were transferred into 15-ml conical tubes. The spheroids were centrifuged at 250 G for 5 minutes at room temperature and the supernatant was aspirated. 1 ml Media A was added to each well of the 6-well low adhesion plates and the plates were incubated in 37° C. incubators. The Media A was prepared to include Basal Media: DMEM-F12, Activin A (100 ng/mL), CHIR99021 2 μM, Insulin-, Transferrin-Selenium (ITS-G) (1:1000), KSR (0.05%), Penicillin streptomycin (1:100), PI-103 (25 nM), and NEAA (1:400).

Differentiation of iPSCs into Definitive Endoderm (DE)

Cells were cultured on day 1-2 in Media A and days 3-5 in Media B. The Media A was prepared to include Basal Media: DMEM-F12, Activin A (100 ng/mL), CHIR99021 2 μM, Insulin-, Transferrin-Selenium (ITS-G) (1:1000), KSR (0.05%), Penicillin streptomycin (1:100), PI-103 (25 nM), and NEAA (1:400). The Media B was prepared to include Basal Media: DMEM-F12, Activin A (100 ng/mL), LDN193189 (200 nM), PI-103 (25 nM), Insulin-Transferrin-Selenium (ITS-G) (1:1000), KSR (0.05%), Pen strep (1:200), NEAA (1:400).

Differentiation of Definitive Endoderm (DE) into Anterior Foregut Endoderm (AFE)

On day 6 the supernatant and spheroids were transferred into 15-ml conical tubes. The spheroids were centrifuged at 250 G for 5 minutes at room temperature and the supernatant was aspirated. The spheroids were washed in PBS and resuspended in 1 ml of AFE media and plated onto 6-well low adhesion plates. The cells were retained as aggregates in suspension in this experiment. AFE media was prepared to include Basal Media: DMEM-F12, LDN193189 (200 nM), SB431542(10 μM), FGF8b (50 ng/ml), Ascorbic Acid (10 μM), Pen Strep (1:200), B27 (without RA) (1:200), N2 (1:100), Glutamax (1:100), BME (1:100), NEAA (1:400), ITS 36 (1:1000), KSR (0.05%).

### Differentiation of Anterior Foregut Endoderm (AFE) to Ventral Pharyngeal Endoderm (VPE)

24-well plates coated with Geltrex (1:100) placed in room temperature for 1 hour. On day 9 the supernatant alongside spheroids were transferred into 15-ml conical tubes. The spheroids were centrifuged at 250 G for 5 minutes at room temperature and the supernatant was aspirated. The spheroids were washed in PBS. 250 μl of VPE1 media was added to each well coated with Geltrex. VPE1 media was prepared to include Basal Media: DMEM-F12, SB431542 (10 uM), FGF8b (50 ng/ml), Retinoic Acid (0.1 μM), Ascorbic Acid (10 μM), Insulin-Transferrin-Selenium (ITS-G) (1:1000), KSR (0.05%), Pen strep (1:200), B27 (without VIT A) (1:200), Glutamax (1:100), BME (1:100), N2(1:100), and NEAA (1:400). On day 10, equal amount of freshly made VPE1 media (500 ul) was added gently without disturbing the aggregates. On day 11, 50% of supernatant was removed and freshly prepared VPE1 media (500 ul) was added per well.

On day 12-13, VPE2 media was added. VPE2 media was prepared to include Basal Media: DMEM-F12, Noggin (50 ng/ml), CHIR99021 (2 μM), FGF8b (50 ng/ml), Retinoic Acid (0.1 μM), Ascorbic Acid (10 μM), Insulin-Transferrin-Selenium (ITS-G) (1:1000), KSR (0.0025%), Pen strep (1:200), NEAA (1:400), B27 (without VIT A) (1:200), Glutamax (1:100), BME (1:100), and N2(1:100). On Day 13, equal amount of freshly made VPE2 media (500 ul) was added gently without disturbing the aggregates.

### Differentiation of Ventral Pharyngeal Endoderm (VPE) to Thymic Epithelial Progenitors (TEP)

On day 14-17, cells were cultured in TEP media. TEP media was prepared to include Basal Media: DMEM-F12, FGF10 (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), and NEAA (1:400).

On day 15, equal amount of freshly made TEP media (500 μl) was added gently without disturbing the aggregates. On day16 and 17, 50% of supernatant was removed and freshly prepared TEP media (500 μl) was added per well.

To further differentiate the cells to thymic epithelial cells (TECs), the supernatant was removed and the cells were cultured in TEC1 media containing Basal Media: DMEM-F12, FGF10 (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), NEAA (1:400), Glutamax (100×), and BME (100×). Day18 &19, 50% of supernatant was removed and freshly prepared TEC1 media (500 ul) was added per well. On day 20-22, TEC2 media containing RANKL was added. TEC2 media was prepared to include Basal Media: DMEM-F12, FGF10 (50 ng/ml), FGF7/KGF (50 ng/ml), RANKL/TRANCE (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), NEAA (1:400), Glutamax (100×), and BME (100×).

On day23, the cells were treated with Accutase and were frozen using Hypothermosol-FRS Media.

### Example 27: Differentiation of iPSC Cells to Thymic Cells (Experiment 33)

Expansion of iPSC Line in Suspension Culture

On day 0, mature Fuji iPS-106 cells (3 to 4 days in suspension; 300-400 microns in diameter) were treated with Accutase to turn aggregates into single cells. 2.5 million single iPSCs were placed in suspension in 6-well ultra-low adhesion plates and cultured in use Stem scale media along with rock inhibitor Y27632(10 μM). On day 1, 1 ml supernatant was removed from the plates and add 1 ml of pre-warmed stem scale media. On day 2, the supernatant alongside spheroids were transferred into 15-ml conical tubes. The spheroids were centrifuged at 250 G for 5 min at room temp and aspirate the supernatant. The spheroids were washed with PBS, resuspended in Media A, Media A was prepared to include, Basal Media: DMEM-F12, Activin A (100 ng/mL), CHIR99021 2 μM, Insulin-Transferrin-Selenium (ITS-G) (1:1000), KSR (0.05%), Pen strep (1:100), PI-103 (25 nM), and NEAA (1:400).

Differentiation of iPSCs into Definitive Endoderm (DE)

On day 1-2, cells were cultured in Media A which contained, Basal Media: DMEM-F12, Activin A (100 ng/mL), CHIR99021 2 μM, Insulin-Transferrin-Selenium (ITS-G) (1:1000), KSR (0.05%), Pen strep (1:100), PI-103 (25 nM), and NEAA (1:400). On day2, 1 ml supernatant from each well was replaced with 1 ml freshly prepared Media A.

On day 3 cells were washed and resuspended in Media B which was prepared to include Basal Media: DMEM-F12, Activin A (100 ng/mL), CHIRR9901 (2 μM), LDN193189 (200 nM), PI-103 (25 nM), Insulin-Transferrin-Selenium (ITS-G) (1:1000), KSR (0.05%), Pen strep (1:200), NEAA (1:400). On day 4-5, 1 ml supernatant from each well was replaced with 1 ml freshly prepared Media B′. Media B′ was prepared to include Basal Media: DMEM-F12, Activin A (100 ng/mL), LDN193189 (200 nM), PI-103 (25 nM), Insulin-Transferrin-Selenium (ITS-G) (1:1000), KSR (0.05%), Pen strep (1:200) and NEAA (1:400).

Differentiation of Definitive Endoderm (DE) into Anterior Foregut Endoderm (AFE)

On day 6, 24-well plates were coated with Geltrex (1:100) placed in room temperature for 1 hour. Supernatants alongside spheroids were transferred into 15 ml conical tubes. The spheroids were centrifuged at 250 G for 5 min at room temperature and the supernatant was aspirated. The spheroids were washed with PBS and resuspended in AFE media and transferred to Geltrex coated plates. The AFE media was prepared to include Basal Media: DMEM-F12, LDN193189 (200 nM), SB431542(10 μM), FGF8b (50 ng/ml), Ascorbic Acid (10 μM), Pen Strep (1:200), B27 (without RA) (1:200), N2 (1:100), Glutamax (1:100), BME (1:100), and NEAA (1:400).

On day 7, equal amount of freshly prepared AFE media (500 ul) was added per well. On day8, 50% of supernatant was removed and freshly prepared AFE media (500 ul) was added per well.

### Differentiation of Anterior Foregut Endoderm (AFE) to Ventral Pharyngeal Endoderm (VPE)

On days 9-10, cells were resuspended in VPE1 media containing Basal Media: DMEM-F12, SB431542 (10 uM), FGF8b (50 ng/ml), Retinoic Acid (0.1 μM), Ascorbic Acid (10 μM), Insulin-Transferrin-Selenium (ITS-G) (1:1000), KSR (0.05%), Pen strep (1:200), B27 (without VIT A) (1:200), Glutamax (1:100), BME (1:100), N2(1:100), NEAA (1:400). On day 9, equal amount of freshly made VPE1 media (500 ul) was replaced gently without disturbing the aggregates. On day 10, equal amount of freshly prepared VPE1 media (500 ul) was added per well.

On day 12, equal amount of freshly made VPE2 media (500 ul) was replaced gently without disturbing the aggregates. VPE2 media was prepared to include Basal Media: DMEM-F12, Noggin (50 ng/ml), CHIR99021 (2 μM), FGF8b (50 ng/ml), Retinoic Acid (0.1 μM), Ascorbic Acid (10 μM), Insulin-Transferrin-Selenium (ITS-G) (1:1000), KSR (0.0025%), Pen strep (1:200), NEAA (1:400), B27 (without VIT A) (1:200), Glutamax (1:100), BME (1:100), N2(1:100). On day 13, equal amount of freshly prepared VPE2 media (500 ul) was added per well.

### Differentiation of Ventral Pharyngeal Endoderm (VPE) to Thymic Epithelial Progenitors (TEP)

On day14, equal amount of freshly made TEP media (500 ul) was added gently without disturbing the aggregates. TEP media was prepared to contain Basal Media: DMEM-F12, FGF10 (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), and NEAA (1:400).

On day 15, equal amount of freshly prepared TEP media (500 μl) was added per well. On day16-18, 50% of supernatant was removed and freshly prepared TEP media (500 ul) added to each well.

On days 19-22, TEC media was added. TEC media was prepared to include Basal Media: DMEM-F12, FGF10 (50 ng/ml), FGF7/KGF (50 ng/ml), RANKL/TRANCE (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), NEAA (1:400), Glutamax (100×) and BME (100×).

On day22, the cells were treated with Accutase and were frozen using Hypothermosol-FRS Media. The population of cells was split into several experimental conditions. In 33FT1 (7.5M per plate) cells were thawed in 3D and maintained in TEC media (Y27-10 uM on day1) for 6 days. Different concentration and pulses of Survivin inhibitor (YM155) were evaluated. In 33FT2 (7.8M per plate), cells were thawed in 3D and maintained in TEC media (Y27-10 uM on day1) for 6 days. Different concentration and pulses of Survivin inhibitor (YM155) were evaluated. On day6, Group 3 and Group 4 cells were chosen to be transplanted into 5 animals. In 33FT3 (5M per plate), cells were thawed in 2D (Geltrex-coated 24 well plates) and was maintained in TEC media (Y27-10 uM on day1) for 5 days. Different concentration and pulses of Plurisin #1 were evaluated. On day5, they were resuspended into 3D and were maintained in TEC media for 5 days. On day5 they were treated with 20 μM of Plurisin #1. In 33FT4 (7.6M per plate), cells were thawed in 3D and maintained in TEC media (Y27-10 uM on day1) for 5 days. On Day 5, one group treated with 20 nM YM155 and another group was treated with Plurisin #1. In 33FT6 (8M per plate), cells were thawed in 3D and maintained in TEC media (Y27-10 uM on day1) for 6 days. On Day 6, they were treated with 20 μM Plurisin #1 (36 hours) and were transplanted on day7 into 5 animals. In 33FT7 (7.8M per plate), cells were thawed in 3D and maintained in TEC media (Y27-10 uM for day1) for 6 days. On Day6, they were treated with 20 μM Plurisin #1 (24 hours) and were transplanted on day 7 into 2 animals.

### Example 28: Differentiation of iPSC Cells to Thymic Cells (Experiment 37)

Expansion of iPSC Line in Suspension Culture

On day 0, mature iPSC line 18945 (3 to 4 days in suspension; 300-400 microns in diameter) was treated using Accutase to prepare single cells from aggregates. 3 million single iPSCs were placed in suspension in 6-well ultra-low adhesion plates in Stem scale media along with ROCK inhibitor Y27632 (10 μM). On first day, 1 ml supernatant was removed from the plates and 1 ml of pre-warmed Stem scale media was added. On day 2, the supernatant alongside spheroids were transferred into 15-ml conical tubes and resuspended in Media A in 6-well low adhesion plates.

Differentiation of iPSCs into Definitive Endoderm (DE)

On day1, cells were washed with PBS and were resuspended in Media A. Media A was prepared to include Basal Media: DMEM-F12, Activin A (100 ng/mL), CHIR99021 2 μM, Insulin-Transferrin-Selenium (ITS-G) (1:1000), KSR (0.05%), Pen strep (1:100), PI-103 (25 nM), and NEAA (1:400). On day2, 1 ml supernatant from each well was replaced with 1 ml freshly prepared Media A.

On day3, cells were washed with PBS and was resuspended in Media B. Media B was prepared to include Basal Media: DMEM-F12, Activin A (100 ng/mL), CHIRR9901 (2 μM), LDN193189 (200 nM), PI-103 (25 nM), Insulin-Transferrin-Selenium (ITS-G) (1:1000), KSR (0.05%), Pen strep (1:200), and NEAA (1:400).

On day 4-5, 1 ml supernatant from each well was replaced with 1 ml freshly prepared Media B. Media B was prepared to include Basal Media: DMEM-F12, Activin A (100 ng/mL), LDN193189 (200 nM), PI-103 (25 nM), Insulin-Transferrin-Selenium (ITS-G) (1:1000), KSR (0.05%), Pen strep (1:200), and NEAA (1:400).

Differentiation of Definitive Endoderm (DE) into Anterior Foregut Endoderm (AFE)

24-well plates were coated with Geltrex (1:100) placed in room temperature for 1 hour. On day 6 the supernatant alongside spheroids were transferred into 15-ml conical tubes. The spheroids were centrifuged at 250 G for 5 min at room temperature and aspirate the supernatant. The spheroids were washed with PBS and resuspended in AFE media and cultured from day 6-8. AFE media was prepared to include Basal Media: DMEM-F12, LDN193189 (200 nM), SB431542(10 μM), FGF8b (50 ng/ml), Ascorbic Acid (10 μM), Pen Strep (1:200), B27 (without RA) (1:200), N2 (1:100), Glutamax (1:100), BME (1:100), and NEAA (1:400).

On day 7, equal amount of freshly prepared AFE media (500 μl) was added per well. On day 8, 50% of supernatant was removed and freshly prepared AFE media (500 μl) added on each well.

### Differentiation of Anterior Foregut Endoderm (AFE) to Ventral Pharyngeal Endoderm (VPE)

On day 9, equal amount of freshly made VPE1 media (500 ul) was replaced gently without disturbing the aggregates. VPE1 was prepared to include Basal Media: DMEM-F12, SB431542 (10 uM), FGF8b (50 ng/ml), Retinoic Acid (0.1 μM), Ascorbic Acid (10 μM), Insulin-Transferrin-Selenium (ITS-G) (1:1000), KSR (0.05%), Pen strep (1:200), B27 (without VIT A) (1:200), Glutamax (1:100), BME (1:100), N2(1:100) and NEAA (1:400). On day10, equal amount of freshly prepared VPE1 media (500 μl) was added per well.

Day 12, equal amount of freshly made VPE2 media (500 ul) was replaced gently without disturbing the aggregates. VPE2 media was prepared to include Basal Media: DMEM-F12, Noggin (50 ng/ml), CHIR99021 (2 μM), FGF8b (50 ng/ml), Retinoic Acid (0.1 μM), Ascorbic Acid (10 μM), Insulin-Transferrin-Selenium (ITS-G) (1:1000), KSR (0.0025%), Pen strep (1:200), NEAA (1:400), B27 (without VIT A) (1:200), Glutamax (1:100), BME (1:100), N2(1:100)

On day 13, equal amount of freshly prepared VPE2 media (500 μl) was added per well.

### Differentiation of Ventral Pharyngeal Endoderm (VPE) to Thymic Epithelial Progenitors (TEP)

On day14, equal amount of freshly made TEP media (500 μl) was replaced gently without disturbing the aggregates. The cells were cultured in TEP media from days 14-18. TEP media was prepared to include Basal Media: DMEM-F12, FGF10 (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), and NEAA (1:400). On day 15, equal amount of freshly prepared TEP media (500 ul) was added per well. On days 16-18, 50% of supernatant was removed and freshly prepared TEP media (500 ul) was added per well.

On days 19-22, cells were cultured in TEC media. On day19, equal amount of freshly made TEC media (500 μl) was replaced gently without disturbing the aggregates. TEC media was prepared to include Basal Media: DMEM-F12, FGF10 (50 ng/ml), IL-22 (20 ng/ml), FGF7/KGF (50 ng/ml), RANKL/TRANCE (50 ng/ml), BMP4 (50 ng/ml), FGF8b (50 ng/ml), CHIRR99 (2 μM), Ascorbic Acid (10 μM), Insulin-Transferrin-Selenium (ITS-G) (1:1000), KSR (0.0025%), Pen strep (1:200), B27 (without VIT A) (1:200), Glutamax (1:100), BME (1:100), N2(1:100), NEAA (1:400), Glutamax (100×), and BME (100×). On day 20, equal amount of freshly prepared TEC media (500 ul) along with Plurisin #1(20 μM) was added (Groups-Untreated and Treated). On day21, equal amount of freshly made TEC media (500 ul) was replaced gently without disturbing the aggregates. On day22, the cells were treated with Accutase and were frozen using Hypothermosol-FRS Media. The cells were split into the following groups for experimentation. Group 37FT1 (8.85M Treated and 9.5M untreated groups) was thawed in 3D and maintained in TEC media (Y27-10 uM on day1) for 6 days. On Day 6, cells were treated with 20 μM Plurisin #1 (24 hours) and were transplanted on day7 into 4 animals. Group 37FT2 (8.85M per plate) was thawed in 3D and maintained in TEC media (Y27-10 uM on day1) for 6 days. On Day 6, cells were treated with 20 μM Plurisin #1 (24 hours) and were transplanted on day7 into 8 animals.

### Example 29: Differentiation of iPSC Cells to Thymic Cells (Experiment 40A, 40B and 41)

iPSCs were differentiated to thymic cells as described below.

Expansion of iPS18945 Line in Suspension Culture

On day 0, mature iPSC cells (3 to 4 days in suspension; 300-400 microns in diameter) were treated with Accutase to turn aggregates into single cells. 3 million single iPSC cells were placed in suspension in 6-well ultra-low adhesion plates in Stem Scale media along with ROCK inhibitor Y27632 (10 μM). The plates were placed on a shaker at 70 RPM at 37° C. On day 1, 1 ml supernatant was removed from the plates and 1 ml of pre-warmed Stem Scale media was added. On day 2, the supernatant alongside spheroids were transferred into 15-ml conical tubes. The spheroids were centrifuged at 250 G for 5 min at room temperature and the supernatant was aspirated. The spheroids were washed with PBS, centrifuged at 250 G for 5 min and the PBS was aspirated. Media A was prepared to include basal media: DMEM-F12, Activin A (100 ng/mL), CHIR99021, (2 μM), Insulin-Transferrin-Selenium (ITS-G) (1:1000), KSR (0.05%), Pen strep (1:100), PI-103 (25 nM), and NEAA (1:400). 1 ml of Media A was added to each well of the 6-well low adhesion plates.

Differentiation of iPSCs into Definitive Endoderm (DE)

Differentiation of iPSCs into DE requires proper timing for introducing small molecules and growth factors. On day 1-2, Media A was added (Media A was prepared to include basal media: DMEM-F12, Activin A (100 ng/mL), CHIR99021, (2 μM), Insulin-Transferrin-Selenium (ITS-G) (1:1000), KSR (0.05%), Pen strep (1:100), PI-103 (25 nM), and NEAA (1:400). On day 3, Media B was added (Media B was prepared to include Basal Media: DMEM-F12, Activin A (100 ng/mL), CHIRR99021 2 μM, LDN193189 (200 nM), PI-103 (25 nM), Insulin-Transferrin-Selenium (ITS-G) (1:500), KSR (0.05%), Pen strep (1:200), NEAA (1:400)). On day 4-5, Media B′ was added (Media B′ was prepared to include basal Media: DMEM-F12, Activin A (100 ng/mL), LDN193189 (200 nM), PI-103 (25 nM), Insulin-Transferrin-Selenium (ITS-G) (1:500), KSR (0.05%), Pen strep (1:200), and NEAA (1:400)).

Differentiation of Definitive Endoderm (DE) into Anterior Foregut Endoderm (AFE)

On day 6, the supernatant alongside spheroids was transferred into 15-ml conical tubes. The spheroids were centrifuged at 250 G for 5 min at room temp and the supernatant was aspirated. The spheroids were washed with PBS, centrifuged at 250 G for 5 min and the PBS was aspirated. 250 ul of AFE Media was added to each well of 24-well plates coated with Geltrex (1:100) placed in room temperature for 1 hour. The AFE media was prepared to include Basal Media: DMEM-F12, LDN193189 (200 nM), SB431542(10 μM), FGF8b (50 ng/ml), Ascorbic Acid (10 μM), Insulin-Transferrin-Selenium (ITS-G) (1:500), KSR (0.05%), Pen Strep (1:200), B27 (without RA) (1:200), N2 (1:100), Glutamax (1:100), BME (1:100), and NEAA (1:400). 6 ml AFE Media was added to the DE-aggregates. Aggregates were gently mixed and add 250 ul to each well of the 24-well plate containing AFE Media. On day 6, media was replaced with fresh AFE media. On day 7, 500 μL of AFE′ media was added. AFE′ media was prepared to include Basal Media: DMEM-F12, LDN193189 (200 nM), SB431542(10 μM), FGF8b (50 ng/ml), Ascorbic Acid (10 μM), Insulin-Transferrin-Selenium (ITS-G) (1:500), KSR (0.05%), Pen Strep (1:200), B27 (without RA) (1:200), N2 (1:100), Glutamax (1:100), BME (1:100), and NEAA (1:400). Experiment 40A and 40B were cultured in AFE′ media for 2 days, whereas in experiment 40B, the cells were cultured in AFE media for 3 days.

### Differentiation of Anterior Foregut Endoderm (AFE) to Ventral Pharyngeal Endoderm (VPE)

Cells were then cultured in VPE1 media shown in Table 4.

TABLE 4
VPE1 Media for Experiment 40A and Experiment 40B

| VPE1 media contents | Experiment 40A | Experiment 40B |
|---|---|---|
| and concentrations | Day 8 to 11 | Day 8 to 11 |
| DMEMF12 | 12 ml | 12 ml |
| FGF8b (50 ng/ml) | 12 μl | 12 μl |
| SB431542 (10 μM) | 12 μl | 12 μl |
| Ascorbic Acid | 12 μl | 12 μl |
| ITS (1:500) | 24 μl | 24 μl |
| KSR (0.05%) | 6 μl | 6 μl |
| NEAA | 30 μl | 30 μl |
| Pen Strep | 60 μl | 60 μl |
| B27 | 60 μl | 60 μl |
| N2 | 120 μl | 120 μl |
| Glutamax | 120 μl | 120 μl |
| BME | 120 μl | 120 μl |
| LDN193189 (200 nM) | 24 μl | 24 μl |
| Retinoic Acid | 12 μl | 12 μl |
| (4 μl + 996DMSO) | | |
| FGF7 | 12 μl | 12 μl |
| FGF10 | 12 μl | 12 μl |
| Wnt inhibitor | 12 μl | — |
| IWR1(2.5 μM) | | |

On Day 10, for Experiment 40A and Experiment 40B equal amount of freshly made VPE 1 media (500 μl) was added gently without disturbing the aggregates.

On day 10, 500 μl freshly prepared VPE1 media was added to the aggregates in Experiment 41.

For Experiment 41, VPE1 media was prepared to include the following: Basal Media: DMEM-F12, All Trans Retinoic Acid (0.1 μM), SB431542(10 μM), FGF8b (50 ng/ml), Ascorbic Acid (10 μM), Insulin-Transferrin-Selenium (JTS-G) (1:500), KSR (0.05%), Pen Strep (1:200), B27 (without RA) (1:200), N2 (1:100), Glutamax (1:100), BME (1:100), NEAA (1:400).

VPE 2 media was added to cells in each experiment as shown below in Table 5.

TABLE 5
VPE2 Media for Experiment 40A, Experiment
40B and Experiment 41

| | Experiment | Experiment | Experiment |
|---|---|---|---|
| VPE2 media contents | 40A Day | 40B Day | 41 Day |
| and concentrations | 12 to 14 | 12 to 14 | 11 and 12 |
| DMEMF12 | 12 ml | 12 ml | 24 ml |
| FGF8b (50 ng/ml) | 12 μl | 12 μl | 24 μl |
| SB431542 (10 uM) | — | — | 24 μl |
| LDN193189 (200 nM) | — | — | 48 μl |
| Ascorbic Acid | 12 μl | 12 μl | 24 μl |
| ITS (1:500) | 24 μl | 24 μl | 48 μl |
| KSR | 3 μl | 3 μl | 6 μl |
| NEAA | 30 μl | 30 μl | 60 μl |
| Pen Strep | 60 μl | 60 μl | 120 μl |
| B27 | 60 μl | 60 μl | 120 μl |
| N2 | 120 μl | 120 μl | 240 μl |
| Glutamax | 120 μl | 120 μl | 240 μl |
| BME | 120 μl | 120 μl | 240 μl |
| CHIRR99021 | 12 μl | 12 μl | 24 μl |
| Retinoic Acid | — | — | 24 μl |
| (4 ul + 996 ul DMSO) | | | |
| BMP4 | 12 μl | 12 μl | — |
| SANT-1 (SHH | 12 μl | — | — |
| antagonist) | | | |

Expression of HOXA3 and PAX9 at the end of the VPE stage was measured. FIG. 15 shows that Experiment 40A and Experiment 40B resulted in higher level of HOXA3 and Pax9 compared to Experiment 41.

### Differentiation of Ventral Pharyngeal Endoderm (VPE) to Thymic Epithelial Progenitors (TEP)

For Experiment 41, TEP media as shown in Table 6 was added on days 13-17. On Day14, equal amount of freshly made TEP media (500 μl) was added gently without disturbing the aggregates. On days15-17, 50% of supernatant was removed and freshly prepared TEP media (500 μl) was added per well.

TABLE 6
TEP Media for Experiment 40A, Experiment
40B and Experiment 41

| | Experiment | Experiment | Experiment |
|---|---|---|---|
| TEP media contents | 40A Day | 40B Day | 41 Day |
| and concentrations | 15-17 | 15-17 | 13-17 |
| DMEMF12 | 12 ml | 12 ml | 24 ml |
| FGF8b (50 ng/ml) | 12 μl | 12 μl | 24 μl |
| Ascorbic | 12 μl | 12 μl | 24 μl |
| Acid (10 μM) | | | |
| ITS (1:500) | 24 μl | 24 μl | 48 μl |
| KSR (0.05%) | 3 μl | 3 μl | 6 μl |
| NEAA (1:400) | 30 μl | 30 μl | 60 μl |
| Pen Strep (1:200) | 60 μl | 60 μl | 120 μl |
| B27 without | 60 μl | 60 μl | 120 μl |
| retinoic acid (1:200) | | | |
| N2 | 120 μl | 120 μl | 240 μl |
| Glutamax (1:100) | 120 μl | 120 μl | 240 μl |
| BME (1:100) | 120 μl | 120 μl | 240 μl |
| CHIRR99021 (2 μM) | 12 μl | 12 μl | 24 μl |
| FGF10 (50 ng/ml) | 12 μl | 12 μl | 24 μl |
| BMP4 (50 ng/ml) | 12 μl | 12 μl | 24 μl |
| FGF7 | 12 μl | 12 μl | — |
| SANT-1 (SHH antagonist) | 12 μl | — | — |

Day 16, equal amount of freshly prepared TEP media (500 μl) was added gently without disturbing the aggregates. On Day17, 50% of supernatant was removed and freshly prepared TEP media (500 μl) was added per well.

On day 18, the aggregates were transferred to TEC media. The TEC media was prepared to include basal Media: DMEM-F12, FGF10 (50 ng/ml), FGF7/KGF (50 ng/ml), RANKL/TRANCE (50 ng/ml), BMP4 (50 ng/ml), FGF8b (50 ng/ml), CHJRR99021 (2 μM), JL-22 (20 nM), Ascorbic Acid (10 μM), Insulin-Transferrin-Selenium (ITS-G) (1:1000), KSR (0.0025%), Pen strep (1:200), B27 (without Vitamin A) (1:200), Glutamax (1:100), BME (1:100), N2(1:100), NEAA (1:400), Glutamax (100×), BME (100×).

On day19, 50% of supernatant was removed and freshly prepared TEC media (500 μl) was added per well. On day20, 20 μM PluriSIn-1 (a stearoyl-CoA desaturase inhibitor), was added to the freshly prepared media. On day21, the TEC media without PluriSIn-1 was added. On day22, the cells were treated with enzyme Accutase to generate single cells which were frozen prior to further testing and experimentation.

### Example 30: Reproducibility of Thymic Cell Differentiation Protocol

A new iPSC line was tested using differentiation protocol Experiment 37. For each experiment FOXN1 expression in iPSC derived TEPs was measured at the end of differentiation before freezing the cells, and 5 days after thawing the cells into 3D aggregate. A marked increase in FOXN1 expression was observed in thawed cells compared to FOXN1 expression before freezing the iPSC derived thymic cells. This experiment demonstrates that the differentiation protocol was easily transferred across iPSC cell line and that the freezing/thawing process can consistently upregulate FOXN1 expression.

### Example 31: Elimination of Undifferentiated Pluripotent Stem Cells from TEP Populations

An important safety issues to address in iPSC-derived cell therapy products was the possible presence of residual undifferentiated pluripotent cells in the end. Such iPSCs in the product can give rise to teratomas. To eradicate residual iPSCs, small molecules that have been shown to selectively target pluripotent stem cells utilized. YM155, a Survivin inhibitor and PluriSIn-1, a stearoyl-CoA desaturase inhibitor was tested. TEP population treated with YM155 prior to transplantation resulted in smaller teratomas in vivo compared to animals that transplanted with TEPs that were not treated with YM155.

Treatment with PluriSIn-1 (one 24-hour pulse before freezing and one 24-hr pulse after thawing in aggregate) resulted in TEP populations with lower Oct4 and Nanog expression compared to populations treated with one pulse of PluriSIn-1. However, treatment with one dose of PluriSIn-1 alone was sufficient to reduce Oct4 and Nanog expression when a different iPSC cell line was used in the generation of TEPs.

Frozen cells from Experiment 29B were thawed into aggregates for 5 days and treated with YM155 for 24 hours before transplantation into sub renal capsule of NSG dKO mice transplanted with huCD34+ cells 2 weeks before. Animals were tracked and bled every 3 weeks and peripheral blood mononuclear cells were analyzed by flow cytometry for human hematopoietic cells (CD45), B cells (CD19), myeloid cells (CD19), T cells (CD3/CD4 and CD3/CD8) and mature T cells receptor (TCRa and b). All animals demonstrated robust repopulation of PBMC by human CD45+ hematopoietic cells by 5 weeks after transplantation. Using double staining for CD3 and CD4 or CD8, single positive T cells in peripheral blood circulation was measured. One out of 2 animals transplanted with fetal thymus developed CD4 and CD8 T cells by week 8-9 cells. One out of 6 animals transplanted with iPSC derived TEP developed T cells by week 9, with level of CD4 slightly higher than CD8. The T cells in these experiments also expressed mature a and b T cell receptors. Control sham transplanted animal continue to show basal level of T cells below 1% at 13 weeks.

### EQUIVALENTS AND SCOPE

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” can mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art can be explicitly excluded from any one or more of the claims. Since such embodiments are deemed to be known to one of ordinary skill in the art, they can be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes can be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method of differentiating pluripotent stem cells to thymic cells, comprising:
a) differentiating the pluripotent stem cells into definitive endoderm (DE) cells;
b) culturing the DE cells and differentiating the DE cells into anterior foregut endoderm (AFE) cells by contacting or incubating the DE cells with a BMP inhibitor, a TGFβ inhibitor, an FGF, an ascorbic acid or a combination thereof;
c) culturing the AFE cells and differentiating the anterior foregut cells into ventral pharyngeal endoderm (VPE) cells comprising:
i) contacting or incubating the AFE cells in a first VPE medium comprising ascorbic acid, a retinoic acid, an FGF, a TGFβ inhibitor or a combination thereof; and
ii) contacting or incubating the AFE cells in a second VPE medium comprising a Noggin, a WNT activator, an FGF, a retinoic acid, an ascorbic acid or a combination thereof; and
d) culturing the VPE cells and differentiating the VPE cells into thymic cells, by contacting or incubating the VPE cells with an ascorbic acid, an FGF, a BMP, a WNT activator or a combination thereof;
wherein the thymic cells are thymic epithelial progenitor (TEP) cells.

2. The method of claim 1, wherein the TEPs are further differentiated into thymic epithelial cells (TECs) by contacting or incubating the TEPs with an Interleukin, a WNT activator, a RANKL, an FGF, a BMP, an ascorbic acid, or a combination thereof.

3. The method of claim 1, wherein the differentiation of pluripotent stem cells to DE cells comprises
a) contacting or culturing the pluripotent stem cells in a first growth medium, said first growth medium comprising Activin A, PI-103, CHIR99021 or a combination thereof;
b) culturing the pluripotent stem cells in a second growth medium to generate definitive endoderm cells, wherein the second growth medium comprises an, Activin A, a BMP inhibitor, PI-103, CHIR99021 or a combination thereof.

4. The method of claim 1, wherein the first VPE media in step c)i) further comprises a WNT inhibitor.

5. The method of claim 1, wherein the second VPE media in step c)ii) further comprises a BMP inhibitor, an SHH inhibitor, or a combination thereof.

6. A method of differentiating pluripotent stem cells to thymic cells, comprising:
a) differentiating the pluripotent stem cells into definitive endoderm (DE) cells;
b) culturing the DE cells and differentiating the DE cells into anterior foregut endoderm (AFE) cells by contacting or incubating the DE cells with a BMP inhibitor, a TGFβ inhibitor, an FGF, an ascorbic acid or a combination thereof, wherein the BMP inhibitor is LDN193189;
c) culturing the AFE cells and differentiating the anterior foregut cells into ventral pharyngeal endoderm (VPE) cells comprising:
i) contacting or incubating the AFE cells in a first VPE medium comprising ascorbic acid, a retinoic acid, an FGF, a TGFβ inhibitor or a combination thereof, wherein the TGFβ inhibitor is SB431542, and wherein the FGF is FGF8b, FGF7, FGF10, FGF1, bFGF or a combination thereof; and
ii) contacting or incubating the AFE cells in a second VPE medium comprising a Noggin, a WNT activator, an FGF, a retinoic acid, an ascorbic acid or a combination thereof, wherein the WNT activator is CHIR99021; and
d) culturing the VPE cells and differentiating the VPE cells into thymic cells, by contacting or incubating the VPE cells with an ascorbic acid, an FGF, a BMP, a WNT activator or a combination thereof, wherein the BMP is BMP2, BMP4 or a combination thereof;
wherein the thymic cells are thymic epithelial progenitor (TEP) cells.

7-10. (canceled)

11. The method of claim 1, wherein the interleukin is IL22.

12. (canceled)

13. The method of claim 5, wherein the BMP inhibitor is LDN193189.

14. The method of claim 5, wherein the SHH inhibitor is SANT-1.

15. The method of claim 1, wherein the pluripotent stem cells, the DE cells, the AFE cells, the VPE cells or thymic cells are cultured in 3D culture.

16. The method of claim 15, wherein the pluripotent stem cells, the DE cells, the AFE cells, the VPE cells or thymic cells are cultured as aggregates in suspension.

17. The method of claim 1, wherein the method is performed for from about 15 days to 30 days.

18. (canceled)

19. The method of claim 1, wherein the pluripotent stem cells are differentiated into definitive endoderm cells for about 5 days.

20. The method of claim 1, wherein the DE cells are differentiated into AFE cells for from about 2 days to 3 days.

21. The method of claim 1, wherein the AFE cells are cultured in the first VPE media for about 2 to 4 days.

22. The method of claim 1, wherein the AFE cells are cultured in the second VPE media for about 2 to 3 days.

23. The method of claim 1, wherein the VPE cells are differentiated to thymic cells for about 3 to 6 days.

24. The method of claim 2, wherein the TEPs are differentiated to TECs for about 4 days.

25. A population of thymic cells prepared according to the method of claim 1.

26. A pharmaceutical composition comprising a population of thymic cells of claim 25 and at least one excipient.

27. A method of treating or preventing a condition in a subject comprising administering to the subject, the pharmaceutical composition of claim 26.

28. The method of claim 27, wherein the condition is a condition associated with an absence, decline or aberrant functioning of the thymus of the subject, an immunodeficiency, a cancer, an autoimmune disease, an infectious disease, or graft versus host disease (GvHD).

29. The method of claim 27, wherein the pharmaceutical composition is administered to the subject via a parenteral route.

30. The method of claim 27, wherein the pharmaceutical composition is implanted or injected into one or more lymph nodes of the subject.

31. A method of increasing FOXN1 expression in a population of thymic cells, the method comprising
a) freezing the population of thymic cells;
b) thawing the population of thymic cells; and
c) measuring and comparing the expression of FOXN1 in the population of thymic cells prior to freezing and comparing with FOXN1 expression after thawing the population of thymic cells.

32-35. (canceled)
